(19) United States
(12) Patent Application Publication
(10) Pub. No.: US 2022/0119821 A1
(43) Pub. Date: Apr. 21, 2022

(54) RECOMBINANT HOST CELLS AND METHODS FOR THE PRODUCTION OF ASPARTIC ACID AND B-ALANINE

(71) Applicant: Lygos, Inc., Berkeley, CA (US)

(72) Inventors: Jeffrey A. Dietrich, Berkeley, CA (US); Mario Ouellet, Berkeley, CA (US); Ee-Been Goh, Berkeley, CA (US); Chi Le, Berkeley, CA (US); Kelvin Shing, Berkeley, CA (US); Drew Ross, Berkeley, CA (US); Daniel Blackburn, Berkeley, CA (US)

(21) Appl. No.: 16/973,270

(22) PCT Filed: Jun. 24, 2019
(86) PCT No.: PCT/US2019/038732

§ 371 Date: Dec. 8, 2020

Related U.S. Application Data
(60) Provisional application No. 62/689,265, filed on Jun. 25, 2018.

(51) Int. Cl. C12N 15/52; C12N 9/06; C12N 9/10; C12N 9/88; C12N 9/00; C12P 13/06; C12P 13/20; C12N 1/02; C12N 1/20; C12N 9/02

(52) U.S. Cl. CPC C12N 15/52; C12Y 604/01001; C12N 9/1096; C12N 9/88; C12N 9/93; C12P 13/06; C12P 13/20; C12N 1/02; C12N 1/20; C12N 9/001; C12Y 104/01021; C12Y 206/01001; C12Y 401/01031; C12Y 401/01049; C12Y 401/01011; C12Y 103/05001; C12N 9/0016

(57) ABSTRACT

Methods and materials related to producing aspartic acid, β-alanine and salts of each thereof are disclosed. Specifically, isolated nucleic acids, polypeptides, host cells, methods and materials for producing aspartic acid by direct fermentation from sugars are disclosed.

### PRIORITY CLAIM

This application claims priority to U.S. provisional application No. 62/689,265, filed Jun. 25, 2018, the content of which is incorporated herein in its entirety by reference.

### GOVERNMENT INTEREST

This invention was made with government support under award number DE-EE0007565 awarded by the United States Department of Energy. The government has certain rights to the invention.

### REFERENCE TO SEQUENCE LISTING

This application contains a Sequence Listing submitted via EFS-web which is hereby incorporated by reference in its entirety for all purposes. The ASCII copy, created on Jun. 19, 2019, is named Lygos_0016_01_WO_ST25.txt and is 196 KB in size.

### BACKGROUND OF THE INVENTION

Aspartic acid is produced according to an inefficient enzymatic batch process from 1973 that uses immobilized aspartase-rich E. coli cell extracts to convert ammonia and fumaric acid to aspartic acid. Historically used to produce the artificial food sweetener aspartame, aspartic acid has great potential as polyaspartic acid in various applications such as weatherproof and corrosion prevention coatings, household and construction dispersants, biodegradable monolayers for water conservation, and superabsorbent gels for diaper or dressings. Unfortunately, the incumbent process cannot produce high yields to support new market growth. Thus, there is a need for new low-cost, energy efficient, high yielding manufacturing methods.

Similarly, β-alanine is produced by reacting aspartic acid with immobilized aspartate β-decarboxylase-rich Pseudomonas dacunhae cell extracts. β-alanine is a non-essential amino acid used as a performance-enhancing supplement in the sports nutrition market.

The present disclosure provides recombinant host cells and methods to produce aspartic acid and β-alanine by microbial fermentation using a sugar feedstock. Aspartic acid and β-alanine production according to various embodiments of the present disclosure utilizes an efficient overall carbon-conversion route; in cases where glucose is used as the raw material, the stoichiometric theoretical yield is 2 mols of aspartic acid or 2 mols of β-alanine for every mol of glucose. In some embodiments, CO₂ fixation is a feature in the biosynthetic pathway, enabling the upcycling of industrial CO₂ waste. The materials and methods described herein comprise a renewable and low-cost starting material and an environmentally beneficial biosynthetic process.

### SUMMARY OF THE INVENTION

In a first aspect, the invention provides recombinant host cells capable of producing aspartic acid comprising one or more heterologous nucleic acids that encode the aspartic acid biosynthetic pathway, wherein the aspartic acid biosynthetic pathway enzymes comprise an oxaloacetate-forming enzyme and an aspartate-forming enzyme. The invention also provides recombinant host cells capable of producing β-alanine comprising one or more heterologous nucleic acids that encode the β-alanine biosynthetic pathway, wherein the β-alanine biosynthetic pathway enzymes comprise an oxaloacetate-forming enzyme, an aspartate-forming enzyme, and a β-alanine-forming enzyme. In some embodiments, the recombinant host cell is a bacterial cell. In some embodiments, the bacterial cell is Escherichia coli, Corynebacterium glutamicum, or Pantoea ananatis.

In some embodiments, the oxaloacetate-forming enzyme is a pyruvate carboxylase, a phosphoenolpyruvate carboxylase, or a phosphoenolpyruvate carboxykinase. In some embodiments, the recombinant host cells comprise heterologous nucleic acids encoding an oxaloacetate-forming enzyme with at least 40% homology to SEQ ID NO: 12, SEQ ID NO: 13, SEQ ID NO: 14, SEQ ID NO: 15, SEQ ID NO: 16, SEQ ID NO: 17, SEQ ID NO: 18, or SEQ ID NO: 35.

In some embodiments, the aspartate-forming enzyme is an aspartate dehydrogenase or an aspartate transaminase. In some embodiments, the recombinant host cells comprise heterologous nucleic acids encoding an aspartate-forming enzyme with at least 40% homology to SEQ ID NO: 9, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 33, or SEQ ID NO: 36.

In some embodiments, the β-alanine-forming enzyme is an aspartate 1-decarboxylase. In some embodiments, the recombinant host cells comprise heterologous nucleic acids encoding an aspartate 1-decarboxylase with at least 40% homology to SEQ ID NO: 29, SEQ ID NO:37, SEQ ID NO: 38, SEQ ID NO: 39, or SEQ ID NO: 40.

In a second aspect, the invention provides recombinant host cells that further genetic disruption of one or more genes, wherein the one or more genes encodes a lactate dehydrogenase, a succinate dehydrogenase subunit, or a combination thereof. In some embodiments, the one or more genes has at least 40% homology to SEQ ID NO: 1, SEQ ID NO: 2, SEQ ID NO: 10, SEQ ID NO: 11, or any combination thereof.

In a third aspect, the invention provides a method for culturing the recombinant host cells for a sufficient period of time to produce aspartic acid or β-alanine. In some embodiments, the method further comprises anaerobic fermentation. In some embodiments, the method produces an aspartic acid or β-alanine yield of at least 25% g-aspartic acid or β-alanine per g-glucose.

In a fourth aspect, the invention provides a method for the recovery of aspartic acid or β-alanine from the fermentation broth

In another aspect, provided herein is a method for isolating aspartic acid or a salt thereof, comprising:

culturing a recombinant host cell utilized herein in a fermentation broth to produce aspartic acid or a salt thereof;

separating the host cell from the fermentation broth, preferably by centrifugation to produce a clarified fermentation broth;

optionally concentrating the clarified fermentation broth to provide a concentrated fermentation broth;

optionally contacting the concentrated fermentation broth with an ion exchange resin or activated carbon adsorbent;

acidifying the clarified or concentrated fermentation broth to precipitate the aspartic acid or the salt thereof; and

isolating the precipitated aspartic acid or aspartic acid salt.

In one embodiment, the fermentation broth is maintained at a pH of about 6 to about pH 8. In another embodiment, after acidifying, the clarified fermentation broth is concentrated by removing volatile liquids. E.g., volatiles such as water can be distilled out to provide a concentrated fermentation broth. In another embodiment, the clarified fermentation broth is filtered via ultrafiltration or nanofiltration before concentration or acidification. In another embodiment, the concentrated fermentation broth is contacted with an ion exchange resin or activated carbon adsorbent. In another embodiment, the clarified fermentation broth is treated with a decoloring agent such as charcoal. In another embodiment, the acidifying is done with a mineral acid or a resin based acid. Non limiting examples of mineral acids include sulfuric acid, sulfonic acids such as p-toluene sulfonic acid, hydrochloric and other hydrohalic acids, nitric acids, perchloric acids etc. Non limiting examples of resin based acids include polystyrene sulfonic acids and the likes. In another embodiment, the aspartic acid is isolated by filtration. In another embodiment, the supernatant obtained after the crystallization undergoes subsequent crystallization(s) to provide more isolated aspartic acid or a salt thereof. In some embodiments, the isolated aspartic acid or the salt thereof is further purified by recrystallization. The aspartic acid or the salt thereof in the supernatant or the filtrate can be concentrated by one or more of centrifuging, heating, cooling, and filtering. In another embodiment, the fermentation broth comprises at least about 20 g/l of aspartic acid or the salt thereof. In another embodiment, the concentrated acidified broth comprises at least about 70 g/l, at least about 80 g/l, or at least about 90 g/l of aspartic acid or the salt thereof. In another embodiment, up to about 80%, or up to about 90%, or greater than about 90% of the aspartic acid or the salt thereof present in the fermentation broth is isolated. In another embodiment, the isolated aspartic acid or the salt thereof has a purity of about 85%, or about 90%, or more.

In another embodiment, the cell is a bacterial cell. In another embodiment, the cell is Corynebacterium glutamicum. In another embodiment, the cell further comprises: one or more heterologous nucleic acids encoding an aspartate-forming (i.e., an aspartic acid forming) enzyme selected from the group consisting of aspartate dehydrogenase and aspartate transaminase; and one or more heterologous nucleic acids encoding an oxaloacetate-forming enzyme selected from the group consisting of pyruvate carboxylase, phosphoenolpyruvate carboxylase, and phosphoenolpyruvate carboxykinase. In another embodiment, the cells are capable of producing aspartate under anaerobic conditions. In another embodiment, the cells further comprise one or more disruptions of one or more genes encoding a succinate dehydrogenase subunit. In another embodiment, the cells further comprise one or more disruptions of one or more genes encoding a lactate dehydrogenase.

### BRIEF DESCRIPTION OF FIGURES

FIG. 1 provides a schematic of the aspartic acid pathway and the β-alanine pathway enzymes of the present disclosure.

FIG. 2 provides a schematic of a non-limiting and illustrative embodiment of aspartic acid isolation as per the present disclosure.

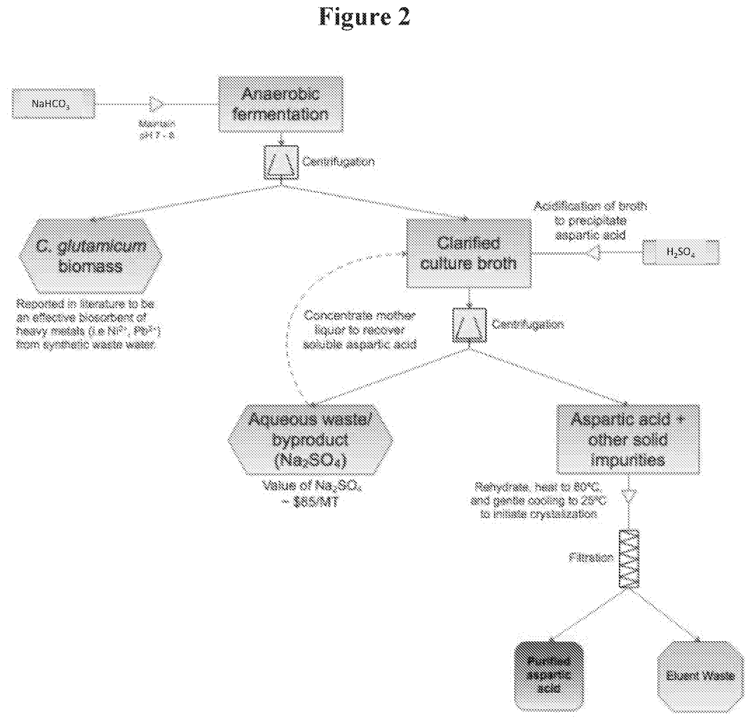

### DETAILED DESCRIPTION

The present disclosure provides recombinant host cells, materials, methods, and embodiments for the biological production and purification of aspartic acid. While the present disclosure describes details specific to L-aspartic acid, those of ordinary skill in the art will recognize that various changes may be made, and equivalents may be substituted without departing from the invention. The present disclosure is not limited to particular nucleic acids, expression vectors, enzymes, biosynthetic pathways, host microorganisms, processes, or enantiomers, as these may vary. The terminology used herein is for the purposes of describing particular embodiments only and is not to be construed as limiting. Because aspartic acid encompasses two different enantiomers—D-aspartic acid (synonymous with R-aspartic acid) and L-aspartic acid (synonymous with S-aspartic acid)—many materials, methods, and embodiments disclosed that relate to L-aspartic acid also pertain to D-aspartic acid. In addition, many modifications may be made to adapt to a particular situation, materials, composition of matter, process, process steps or process flows, in accordance with the invention. All such modifications are within the scope of the claims appended hereto.

### Section 1: Definitions

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols in Molecular Biology (F. M. Ausubel, et al. eds., (1987)); the series Methods in Enzymology (Academic Press, Inc.): PCR 2: A Practical Approach (M. J. MacPherson, B. D. Flames and G. R. Taylor eds. (1995)), Harlow and Lane, eds. (1988) Antibodies, a Laboratory Manual, and Animal Cell Culture (R. I. Freshney, ed. (1987)).

As used in the specification and claims, the singular form “a,” “an” and “the” include plural references unless the context clearly dictates otherwise.

As used herein, the term “comprising” is intended to mean that the compounds, compositions and processes include the recited elements, but not exclude others. “Consisting essentially of” when used to define compounds, compositions and processes, shall mean excluding other elements of any essential significance to the combination. Thus, a composition consisting essentially of the elements as defined herein would not exclude trace contaminants, e.g., from the isolation and purification method. “Consisting of” shall mean excluding more than trace elements of other ingredients. Embodiments defined by each of these transition terms are within the scope of this technology.

All numerical designations, e.g., pH, temperature, time, concentration, and molecular weight, including ranges, are approximations which are varied (+) or (−) by increments of 1, 5, or 10%, e.g., by using the prefix, “about.” It is to be understood, although not always explicitly stated that all numerical designations are preceded by the term “about.” It also is to be understood, although not always explicitly stated, that the reagents described herein are merely exemplary and that equivalents of such are known in the art. As used herein, the range, “about x to y” includes about x to about y.

A “salt” is derived from a variety of organic and inorganic counter ions well known in the art and include, when the compound contains an acidic functionality, by way of example only, sodium, potassium, calcium, magnesium, ammonium, and tetraalkylammonium; and when the molecule contains a basic functionality, salts of organic or inorganic acids, such as hydrochloride, hydrobromide, tartrate, mesylate, acetate, maleate, and oxalate. Salts include acid addition salts formed with inorganic acids or organic acids. Inorganic acids suitable for forming acid addition salts include, by way of example and not limitation, hydrohalide acids (e.g., hydrochloric acid, hydrobromic acid, hydroiodic acid, etc.), sulfuric acid, nitric acid, phosphoric acid, and the like.

Organic acids suitable for forming acid addition salts include, by way of example and not limitation, acetic acid, trifluoroacetic acid, propionic acid, hexanoic acid, cyclopentanepropionic acid, glycolic acid, oxalic acid, pyruvic acid, lactic acid, malonic acid, succinic acid, malic acid, maleic acid, fumaric acid, tartaric acid, citric acid, palmitic acid, benzoic acid, 3-(4-hydroxybenzoyl) benzoic acid, cinnamic acid, mandelic acid, alkylsulfonic acids (e.g., methanesulfonic acid, ethanesulfonic acid, 1,2-ethane-disulfonic acid, 2-hydroxyethanesulfonic acid, etc.), arylsulfonic acids (e.g., benzenesulfonic acid, 4-chlorobenzenesulfonic acid, 2-naphthalenesulfonic acid, 4-toluenesulfonic acid, camphorsulfonic acid, etc.), glutamic acid, hydroxynaphthoic-acid, salicylic acid, stearic acid, muconic acid, and the like.

Salts also include salts formed when an acidic proton present in the parent compound is either replaced by a metal ion (e.g., an alkali metal ion, an alkaline earth metal ion, or an aluminum ion) or by an ammonium ion (e.g., an ammonium ion derived from an organic base, such as, ethanolamine, diethanolamine, triethanolamine, morpholine, piperidine, dimethylamine, diethylamine, triethylamine, and ammonia).

In this specification and in the claims that follow, reference will be made to a number of terms that shall be defined to have the following meanings.

The term “accession number” and similar terms such as “protein accession number”, “UniProt ID”, “gene ID” and “gene accession number” refer to designations given to specific proteins or genes. These identifiers described a gene or protein sequence in publicly accessible databases such as the National Center for Biotechnology Information (NCBI).

The term “heterologous” as used herein refers to a material that is non-native to a cell. For example, a nucleic acid is heterologous to a cell, and so is a “heterologous nucleic acid” with respect to that cell, if at least one of the following is true: 1) the nucleic acid is not naturally found in that cell (that is, it is an “exogenous” nucleic acid); 2) the nucleic acid is naturally found in a given host cell (that is, “endogenous to”), but the nucleic acid or the RNA or protein resulting from transcription and translation of this nucleic acid is produced or present in the host cell in an unnatural (e.g., greater or lesser than naturally present) amount; 3) the nucleic acid comprises a nucleotide sequence that encodes a protein endogenous to a host cell but differs in sequence from the endogenous nucleotide sequence that encodes that same protein (having the same or substantially the same amino acid sequence), typically resulting in the protein being produced in a greater amount in the cell, or in the case of an enzyme, producing a mutant version possessing altered (e.g., higher or lower or different) activity; and/or 4) the nucleic acid comprises two or more nucleotide sequences that are not found in the same relationship to each other in the cell. As another example, a protein is heterologous to a host cell if it is produced by translation of RNA or the corresponding RNA is produced by transcription of a heterologous nucleic acid. Further, a protein is also heterologous to a host cell if it is a mutated version of an endogenous protein, and the mutation was introduced by genetic engineering.

The term “homologous”, as well as variations thereof, such as “homology”, refers to the similarity of a nucleic acid or amino acid sequence, typically in the context of a coding sequence for a gene or the amino acid sequence of a protein. Homology searches can be employed using a known amino acid or coding sequence (the “reference sequence”) for a useful protein to identify homologous coding sequences or proteins that have similar sequences and thus are likely to perform the same useful function as the protein defined by the reference sequence. As will be appreciated by those of skill in the art, a protein having homology to a reference protein is determined, for example and without limitation, by a BLAST (https://blast.ncbi.nlm.nih.gov) search. A protein with high percent homology is highly likely to carry out the identical biochemical reaction as the reference protein. In some cases, two enzymes having greater than 40% homology will carry out identical biochemical reactions, and the higher the homology, i.e., 40%, 50%, 60%, 70%, 80%, 90% or greater than 95% homology, the more likely the two proteins have the same or similar function. A protein with at least 60% homology, and in some cases, at least 40% homology, to its reference protein is defined as substantially homologous. Any protein substantially homologous to a reference sequence can be used in a host cell according to the present disclosure.

Generally, homologous proteins share substantial sequence identity. Sets of homologous proteins generally possess one or more specific amino acids that are conserved across all members of the consensus sequence protein class. The percent sequence identity of a protein relative to a consensus sequence is determined by aligning the protein sequence against the consensus sequence. Practitioners in the art will recognized that various sequence alignment algorithms are suitable for aligning a protein with a consensus sequence. See, for example, Needleman, S B, et al., “A general method applicable to the search for similarities in the amino acid sequence of two proteins.” Journal of Molecular Biology 48 (3): 443-53 (1970). Following alignment of the protein sequence relative to the consensus sequence, the percentage of positions where the protein possesses an amino acid described by the same position in the consensus sequence determines the percent sequence identity. When a degenerate amino acid is present (i.e., B, Z, X, J or “+”) in a consensus sequence, any of the amino acids described by the degenerate amino acid may be present in the protein at the aligned position for the protein to be identical to the consensus sequence at the aligned position. When it is not possible to distinguish between two closely related amino acids, the following one-letter symbol is used—“B” refers to aspartic acid or asparagine; “Z” refers to glutamine or glutamic acid; “J” refers to leucine or isoleucine; and “X” or “+” refers to any amino acid.

A dash (−) in a consensus sequence indicates that there is no amino acid at the specified position. A plus (+) in a consensus sequence indicates any amino acid may be present at the specified position. Thus, a plus in a consensus sequence herein indicates a position at which the amino acid is generally non-conserved; a homologous enzyme sequence, when aligned with the consensus sequence, can have any amino acid at the indicated “+” position.

In addition to identification of useful enzymes by percent sequence identity with a given consensus sequence, enzymes useful in the compositions and methods provided herein can also be identified by the occurrence of highly conserved amino acid residues in the query protein sequence relative to a consensus sequence. For each consensus sequence provided herein, a number of highly conserved amino acid residues are described. Enzymes useful in the compositions and methods provided herein include those that comprise a substantial number, and sometimes all, of the highly conserved amino acids at positions aligning with the indicated residues in the consensus sequence. Those skilled in the art will recognize that, as with percent identity, the presence or absence of these highly conserved amino acids in a query protein sequence can be determined following alignment of the query protein sequence relative to a given consensus sequence and comparing the amino acid found in the query protein sequence that aligns with each highly conserved amino acid specified in the consensus sequence.

Proteins that share a specific function are not always defined or limited by percent sequence identity. In some cases, a protein with low percent sequence identity with a reference protein is able to carry out the identical biochemical reaction as the reference protein. Such proteins may share three-dimensional structure which enables shared specific functionality, but not necessarily sequence similarity. Such proteins may share an insufficient amount of sequence similarity to indicate that they are homologous via evolution from a common ancestor and would not be identified by a BLAST search or other sequence-based searches. Thus, in some embodiments of the present disclosure, homologous proteins comprise proteins that lack substantial sequence similarity but share substantial functional similarity and/or substantial structural similarity.

As used herein, the term “express”, when used in connection with a nucleic acid encoding an enzyme or an enzyme itself in a cell, means that the enzyme, which may be an endogenous or exogenous (heterologous) enzyme, is produced in the cell. The term “overexpress,” in these contexts, means that the enzyme is produced at a higher level, i.e., enzyme levels are increased, as compared to the wild-type, in the case of an endogenous enzyme. Those skilled in the art appreciate that overexpression of an enzyme can be achieved by increasing the strength or changing the type of the promoter used to drive expression of a coding sequence, increasing the strength of the ribosome binding site or Kozak sequence, increasing the stability of the mRNA transcript, altering the codon usage, increasing the stability of the enzyme, and the like.

The terms “expression vector” or “vector” refer to a nucleic acid and/or a composition comprising a nucleic acid that can be introduced into a host cell, e.g., by transduction, transformation, or infection, such that the cell then produces (i.e., expresses) nucleic acids and/or proteins other than those native to the cell, or in a manner not native to the cell, that are contained in or encoded by the nucleic acid so introduced. Thus, an “expression vector” contains nucleic acids (ordinarily DNA) to be expressed by the host cell. Optionally, the expression vector can be contained in materials to aid in achieving entry of the nucleic acids into the host cell, such as the materials associated with a virus, liposome, protein coating, or the like. Expression vectors suitable for use in various aspects and embodiments of the present disclosure include those into which a nucleic acid sequence can be, or has been, inserted, along with any preferred or required operational elements. Thus, an expression vector can be transferred into a host cell and, typically, replicated therein (although, on can also employ, in some embodiments, non-replicable vectors that provide for “transient” expression). In some embodiments, an expression vector that integrates into chromosomal, mitochondrial, or plastid DNA is employed. In other embodiments, an expression vector that replicates extrachromasomally is employed. Typical expression vectors include plasmids, and expression vectors typically contain the operational elements required for transcription of a nucleic acid in the vector. Such plasmids, as well as other expression vectors, are described herein or are well known to those of ordinary skill in the art.

The terms “ferment”, “fermentative”, and “fermentation” are used herein to describe culturing microbes under conditions to produce useful chemicals, including but not limited to conditions under which microbial growth, be it aerobic or anaerobic, occurs.

The terms “recombinant host cell” and “recombinant host microorganism” are used interchangeably herein to refer to a living cell that can be (or has been) transformed via insertion of an expression vector. A host cell or microorganism as described herein may be a prokaryotic cell (e.g., a microorganism of the kingdom Eubacteria) or a eukaryotic cell. As will be appreciated by one of skill in the art, a prokaryotic cell lacks a membrane-bound nucleus, while a eukaryotic cell has a membrane-bound nucleus.

The terms “isolated” or “pure” refer to material that is substantially, e.g., greater than 50% or greater than 75%, or essentially, e.g., greater than 90%, 95%, 98% or 99%, free of components that normally accompany it in its native state, e.g., the state in which it is naturally found or the state in which it exists when it is first produced. Additionally, any reference to a “purified” material is intended to refer to an isolated or pure material.

As used herein, the term “nucleic acid” and variations thereof shall be generic to polydeoxyribonucleotides (containing 2-deoxy-D-ribose), polyribonucleotides (containing D-ribose), segments of polydeoxyribonucleotides, and segments of polyribonucleotides. “Nucleic acid” can also refer to any other type of polynucleotide that is an N-glycoside of a purine or pyrimidine base, and to other polymers containing non-nucleotidic backbones, provided that the polymers contain nucleobases in a configuration that allows for base pairing and base stacking, as found in DNA and RNA. As used herein, the symbols for nucleotides and polynucleotides are those recommended by the IUPAC-IUB Commission of Biochemical Nomenclature (Biochem. 9:4022, 1970). A “nucleic acid” may also be referred to herein with respect to its sequence, the order in which different nucleotides occur in the nucleic acid, as the sequence of nucleotides in a nucleic acid typically defines its biological activity, e.g., as in the sequence of a coding region, the nucleic acid in a gene composed of a promoter and coding region, which encodes the product of a gene, which may be an RNA, e.g., a rRNA, tRNA, or mRNA, or a protein (where a gene encodes a protein, both the mRNA and the protein are “gene products” of that gene).

In the present disclosure, the term “genetic disruption” refers to several ways of altering genomic, chromosomal or plasmid-based gene expression. Non-limiting examples of genetic disruptions include CRISPR, RNAi, nucleic acid deletions, nucleic acid insertions, nucleic acid substitutions, nucleic acid mutations, knockouts, premature stop codons and transcriptional promoter modifications. In the present disclosure, “genetic disruption” is used interchangeably with “genetic modification”, “genetic mutation” and “genetic alteration.” Genetic disruptions give rise to altered gene expression and or altered protein activity. Altered gene expression encompasses decreased, eliminated and increased gene expression levels. In some examples, gene expression results in protein expression, in which case the term “gene expression” is synonymous with “protein expression.”

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

As used herein, “recombinant” refers to the alteration of genetic material by human intervention. Typically, recombinant refers to the manipulation of DNA or RNA in a cell or virus or expression vector by molecular biology (recombinant DNA technology) methods, including cloning and recombination. Recombinant can also refer to manipulation of DNA or RNA in a cell or virus by random or directed mutagenesis. A “recombinant” cell or nucleic acid can typically be described with reference to how it differs from a naturally occurring counterpart (the “wild-type”). In addition, any reference to a cell or nucleic acid that has been “engineered” or “modified” and variations of those terms, is intended to refer to a recombinant cell or nucleic acid.

The terms “transduce,” “transform,” “transfect,” and variations thereof as used herein refers to the introduction of one or more nucleic acids into a cell. For practical purposes, the nucleic acid must be stably maintained or replicated by the cell for a sufficient period of time to enable the function(s) or product(s) it encodes to be expressed for the cell to be referred to as “transduced,” “transformed,” or “transfected.” As will be appreciated by those of skill in the art, stable maintenance or replication of a nucleic acid may take place either by incorporation of the sequence of nucleic acids into the cellular chromosomal DNA, e.g., the genome, as occurs by chromosomal integration, or by replication extrachromosomally, as occurs with a freely-replicating plasmid. A virus can be stably maintained or replicated when it is “infective”: when it transduces a host microorganism, replicates, and (without the benefit of any complementary virus or vector) spreads progeny expression vectors, e.g., viruses, of the same type as the original transducing expression vector to other microorganisms, wherein the progeny expression vectors possess the same ability to reproduce.

As used herein, “aspartic acid” is intended to mean the molecule having the chemical formula C₄H₇NO₄ and a molecular mass of 133.11 g/mol (CAS No. 56-84-8). Aspartic acid as described herein can be a salt, acid, base, or derivative depending on the structure, pH and ions present. The terms “aspartic acid” and “aspartate” are used interchangeably.

In conditions with pH values higher than the pKa of aspartic acid (e.g., about pH>3.9 when using a base, such as sodium hydroxide), aspartic acid is deprotonated to the aspartate anion C₄H₆NO₄⁻. Herein, “aspartate anion” is also used interchangeably with “aspartate”, and practitioners skilled in the art understand that these terms are synonyms.

Further, the aspartate anion is capable of forming an ionic bond with a cation to produce an aspartate salt. The term “aspartate” is intended to mean a variety of aspartate salt forms, and is used interchangeably with “aspartate salts”. Non-limiting examples of aspartates comprise sodium aspartate (CAS No. 3792-50-5) and ammonium aspartate (CAS No. 130296-88-7).

Aspartate salts can crystallize in various states of hydration. For example, “sodium aspartate monohydrate” is intended to mean C₄H₈NNaO₅ with a molecular mass of 173.1 g/mol, wherein a single molecule of sodium aspartate crystallizes with one molecule of water. In another example, “magnesium aspartate dihydrate” is intended to mean C₈H₁₆MgN₂O₁₀ with a molecular mass of 324.525 g/mol, wherein a single molecule of magnesium aspartate crystallizes with two molecules of water. Aspartate salts can also form anhydrous crystals; for example, “anhydrous magnesium aspartate” is intended to mean C₈H₁₂MgN₂O₈ with a molecular mass of 288.495 g/mol.

In conditions with pH values lower than the pKa of aspartic acid (e.g., about pH<3.9), the aspartate anion is protonated to form aspartic acid. Herein, “aspartate” is also used interchangeably with “aspartic acid” and practitioners in the art understand that these terms are synonyms.

The aspartic acid and aspartate salts of the present disclosure are synthesized from biologically produced organic components by a fermenting microorganism. For example, aspartic acid, aspartate salts, or their precursor(s) are synthesized from the fermentation of sugars by recombinant host cells of the present disclosure. Practitioners skilled in the art understand that the prefix “bio-” or the adjective “bio-based” may be used to distinguish these biologically-produced aspartic acid and aspartate salts from those that are derived from petroleum feedstocks. As used herein, “aspartic acid” is defined as “bio-based aspartic acid”, and “aspartate salt” is defined as “bio-based aspartate salt”.

As used herein, “β-alanine” is intended to mean the molecule having the chemical formula C₃H₇NO₂ and a molecular mass of 89.09 g/mol (CAS No. 107-95-9). Practitioners of ordinary skill in the art understand that the terms “β-Ala,” “3-aminopropanoate,” and “3-aminopropionic acid” are synonymous with β-alanine and the three terms can be used interchangeably. In conditions with pH values higher than the pKa of β-alanine (e.g., about pH>3.63 when using a base, such as sodium hydroxide), β-alanine is deprotonated to the β-alanine anion C₂H₆NO₂⁻.

Further, the β-alanine anion is capable of forming an ionic bond with a cation to produce an β-alanine salt. The term “β-alanine salt” is intended to mean a variety of β-alanine salt forms.

As used herein, the term “substantially anaerobic” when used in reference to a culture or growth condition is intended to mean the amount of oxygen is less than about 10% of saturation for dissolved oxygen in liquid media. The term is also intended to include sealed chambers of liquid or solid growth medium maintained with an atmosphere of less than about 1% oxygen.

The term “byproduct” or “by-product” means an undesired chemical related to the biological production of a target molecule. In the present disclosure, “byproduct” is intended to mean any amino acid, amino acid precursor, chemical, chemical precursor, organic acid, organic acid precursor, biofuel, biofuel precursor, or small molecule, that may accumulate during biosynthesis of aspartic acid. In some cases, “byproduct” accumulation may decrease the yields, titers or productivities of the target product (e.g., aspartic acid) in a fermentation.

The redox cofactor nicotinamide adenine dinucleotide, NAD, comes in two forms—phosphorylated and un-phosphorylated. The term NAD(P) refers to both phosphorylated (NADP) and un-phosphorylated (NAD) forms, and encompasses oxidized versions (NAD⁺ and NADP⁺) and reduced versions (NADH and NADPH) of both forms. The term “NAD(P)⁺” refers to the oxidized versions of phosphorylated and un-phosphorylated NAD, i.e., NAD⁺ and NADP⁺. Similarly, the term “NAD(P)H” refers to the reduced versions of phosphorylated and un-phosphorylated NAD, i.e., NADH and NADPH. When NAD(P)H is used to describe the redox cofactor in an enzyme catalyzed reaction, it indicates that NADH and/or NADPH is used. Similarly, when NAD(P)⁺ is the notation used, it indicates that NAD⁺ and/or NADP⁺ is used. Those skilled in the art will also appreciate that while many proteins may only bind either a phosphorylated or un-phosphorylated cofactor, there are redox cofactor promiscuous proteins, natural or engineered, that are indiscriminate; in these cases, the protein may use either NADH and/or NADPH. In some embodiments, enzymes that preferentially utilize either NAD(P) or NAD may carry out the same catalytic reaction when bound to either form.

Various values for temperatures, titers, yields, oxygen uptake rate (OUR), and pH are recited in the description and in the claims. It should be understood that these values are not exact. However, the values can be approximated to the rightmost/last/least significant figure, except where otherwise indicated. For example, a temperature range of from about 30° C. to about 42° C. covers the range 25° C. to 44° C. It should be understood that numerical ranges recited can also include the recited minimum value and the recited maximum value when the values are approximated to the rightmost/last/least significant figure. For example, a temperature range of from about 25° C. to about 50° C. covers the range of 25° C. to 50° C.

### Section 2: Recombinant Host Cells for Production of Aspartic Acid and/or β-Alanine

### 2.1 Host Cells

The present disclosure provides recombinant host cells engineered to produce aspartic acid and/or β-alanine, wherein the recombinant host cells comprise one or more heterologous nucleic acids encoding one or more aspartic acid pathway enzymes. In certain embodiments, the recombinant host cells further comprise one or more heterologous nucleic acids encoding one or more ancillary gene products (i.e., gene products other than the aspartic acid and/or β-alanine pathway enzymes) that improve yields, titers and/or productivities of aspartic acid and/or β-alanine. In particular embodiments, the recombinant host cells further comprise disruptions or deletions of endogenous nucleic acids that improve yields, titers and/or productivities of aspartic acid and/or β-alanine. In some embodiments, the recombinant host cells are capable of producing aspartic acid and/or β-alanine under aerobic conditions. In some embodiments, the recombinant host cells are capable of producing aspartic acid and/or β-alanine under substantially anaerobic conditions. The recombinant host cells produce aspartic acid and/or β-alanine at increased titers, yields and productivities as compared to a parental host cell that does not comprise said heterologous nucleic acids.

In some embodiments, the recombinant host cells further comprise one or more heterologous nucleic acids encoding one or more ancillary gene products (i.e., gene products other than the product pathway enzymes) that improve yields, titers and/or productivities of aspartic acid and/or β-alanine. In particular embodiments, the recombinant host cells further comprise disruptions or deletions of endogenous nucleic acids that improve yields, titers and/or productivities of aspartic acid and/or β-alanine. In some embodiments, the recombinant host cells are capable of producing aspartic acid and/or β-alanine under aerobic conditions. In some embodiments, the recombinant host cells are capable of producing aspartic acid and/or β-alanine under substantially anaerobic conditions.

Any suitable host cell may be used in practice of the methods of the present disclosure, and exemplary host cells useful in the compositions and methods provided herein include archaeal, prokaryotic, or eukaryotic cells. In an embodiment of the present disclosure, the recombinant host cell is a prokaryotic cell. In an embodiment of the present disclosure, the recombinant host cell is a eukaryotic cell. In an embodiment of the present disclosure, the recombinant host cell is a C. glutamicum strain. In another embodiment of the present disclosure, the recombinant host cell is an Escherichia coli strain. In yet another embodiment of the present disclosure, the recombinant host cell is a P. ananatis strain. Methods of construction and genotypes of these recombinant host cells are described herein.

In some embodiments, the recombinant host cells are capable of growth and/or production of aspartic acid and/or β-alanine under substantially anaerobic conditions, or the recombinant host cells may be engineered to be capable of growth and/or production of aspartic acid and/or β-alanine under substantially anaerobic conditions.

### 2.1.1 Yeast Cells

In an embodiment of the present disclosure, the recombinant host cell is a yeast cell. Yeast cells are excellent host cells for construction of recombinant metabolic pathways comprising heterologous enzymes catalyzing production of small-molecule products. There are established molecular biology techniques and nucleic acids encoding genetic elements necessary for construction of yeast expression vectors, including, but not limited to, promoters, origins of replication, antibiotic resistance markers, auxotrophic markers, terminators, and the like. Second, techniques for integration/insertion of nucleic acids into the yeast chromosome by homologous recombination are well established. Yeast also offers a number of advantages as an industrial fermentation host. Yeast cells can generally tolerate high concentrations of organic acids and maintain cell viability at low pH and can grow under both aerobic and anaerobic culture conditions, and there are established fermentation broths and fermentation protocols. This characteristic results in efficient product biosynthesis when the host cell is supplied with a carbohydrate carbon source.

In various embodiments, yeast cells useful in the methods of the present disclosure include yeasts of the genera Aciculoconidium, Ambrosiozyma, Arthroascus, Arxiozyma, Ashbya, Babjevia, Bensingtonia, Botryoascus, Botryozyma, Brettanomyces, Bullera, Bulleromyces, Candida, Citeromyces, Clavispora, Cryptococcus, Cystofilobasidium, Debaryomyces, Dekkara, Dipodascopsis, Dipodascus, Eeniella, Endomycopsella, Eremascus, Eremothecium, Erythrobasidium, Fellomyces, Filobasidium, Galactomyces, Geotrichum, Guilliermondella, Hanseniaspora, Hansenula, Hasegawaea, Holtermannia, Hormoascus, Hyphopichia, Issatchenkia, Kloeckera, Kloeckeraspora, Kluyveromyces, Kondoa, Kuraishia, Kurtzmanomyces, Leucosporidium, Lipomyces, Lodderomyces, Malassezia, Metschnikowia, Mrakia, Myxozyma, Nadsonia, Nakazawaea, Nematospora, Ogataea, Oosporidium, Pachysolen, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastosporion, Schizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis, Torulaspora, Trichosporiella, Trichosporon, Trigonopsis, Tsuchiyaea, Udeniomyces, Waltomyces, Wickerhamia, Wickerhamiella, Williopsis, Yamadazyma, Yarrowia, Zygoascus, Zygosaccharomyces, Zygowilliopsis, and Zygozyma, among others.

In various embodiments, the yeast cell is of a species selected from the non-limiting group comprising Candida albicans, Candida ethanolica, Candida guilliermondii, Candida krusei, Candida lipolytica, Candida methanosorbosa, Candida sonorensis, Candida tropicalis, Candida utilis, Cryptococcus curvatus, Hansenula polymorpha, Issatchenkia orientalis, Kluyveromyces lactis, Kluyveromyces marxianus, Kluyveromyces thermotolerans, Komagataella pastoris, Lipomyces starkeyi, Pichia angusta, Pichia deserticola, Pichia galeiformis, Pichia kodamae, Pichia kudriavzevii (P. kudriavzevii), Pichia membranaefaciens, Pichia methanolica, Pichia pastoris, Pichia salicaria, Pichia stipitis, Pichia thermotolerans, Pichia trehalophila, Rhodosporidium toruloides, Rhodotorula glutinis, Rhodotorula graminis, Saccharomyces bayanus, Saccharomyces boulardi, Saccharomyces cerevisiae (S. cerevisiae), Saccharomyces kluyveri, Schizosaccharomyces pombe, and Yarrowia hpolytica. One skilled in the art will recognize that this list encompasses yeast in the broadest sense.

### 2.1.2 Eukaryotic Cells

In addition to yeast cells, other eukaryotic cells are also suitable for use in accordance with methods of the present disclosure, so long as the engineered host cell is capable of growth and/or product formation. Illustrative examples of eukaryotic host cells provided by the present disclosure include, but are not limited to cells belonging to the genera Aspergillus, Crypthecodinium, Cunninghamella, Entomophthora, Mortierella, Mucor, Neurospora, Pythium, Schizochytrium, Thraustochytrium, Trichoderma, and Xanthophyllomyces. Examples of eukaryotic strains include, but are not limited to: Aspergillus niger, Aspergillus oryzae, Crypthecodinium cohnii, Cunninghamella japonica, Entomophthora coronata, Mortierella alpina, Mucor circinelloides, Neurospora crassa, Pythium ultimum, Schizochytrium limacinum, Thraustochytrium aureum, Trichoderma reesei and Xanthophyllomyces dendrorhous.

### 2.1.3 Archaeal Cells

Archaeal cells are also suitable for use in accordance with methods of the present disclosure, and in an embodiment of the present disclosure, the recombinant host cell is an archaeal cell. Illustrative examples of recombinant archaea host cells provided by the present disclosure include, but are not limited to, cells belonging to the genera: Aeropyrum, Archaeglobus, Halobacterium, Methanococcus, Methanobacterium, Pyrococcus, Sulfolobus, and Thermoplasma. Examples of archaea strains include, but are not limited to Archaeoglobus fulgidus, Halobacterium sp., Methanococcus jannaschii, Methanobacterium thermoautotrophicum, Thermoplasma acidophilum, Thermoplasma volcanium, Pyrococcus horikoshii, Pyrococcus abyssi, and Aeropyrum pernix.

### 2.1.4 Prokaryotic Cells

In an embodiment of the present disclosure, the recombinant host cell is a prokaryotic cell. Prokaryotic cells are suitable host cells for construction of recombinant metabolic pathways comprising heterologous enzymes catalyzing production of small-molecule products. Illustrative examples of recombinant prokaryotic host cells include, but are not limited to, cells belonging to the genera Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Pantoea, Phormidium, Pseudomonas, Rhodobacter, Rhodopseudomonas, Rhodospirillum, Rhodococcus, Salmonella, Scenedesmun, Serratia, Shigella, Staphylococcus, Strepromyces, Synnecoccus, Vibrio, and Zymomonas. Examples of prokaryotic strains include, but are not limited to, Bacillus subtilis (B. subtilis), Brevibacterium ammoniagenes, Bacillus amyloliquefacines, Brevibacterium ammoniagenes, Brevibacterium immariophilum, Clostridium acetobutylicum, Clostridium beigerinckii, Corynebacterium glutamicum (C. glutamicum), Enterobacter sakazakii, Escherichia coli (E. coli), Lactobacillus acidophilus, Lactococcus lactis, Mesorhizobium loti, Pantoea ananatis (P. ananatis), Pseudomonas aeruginosa, Pseudomonas mevalonii, Pseudomonas pudita, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, and Staphylococcus aureus, and Vibrio natriegens.

C. glutamicum, E. coli, Vibrio natriegens, and P. ananatis are particularly good prokaryotic host cells for use in accordance with the methods of the present disclosure. C. glutamicum is well utilized for industrial production of various amino acids. Generally regarded as a strict aerobe, while type C. glutamicum is not capable of growth under substantially anaerobic conditions it will catabolize sugar and produce a range of fermentation products. In some embodiments, the recombinant host cell is a C. glutamicum host cell. E. coli is capable of growth and/or product (i.e., aspartic acid and/or β-alanine) formation under substantially anaerobic conditions, is well-utilized in industrial fermentation of small-molecule products, and can be readily engineered. Unlike most wild type yeast strains, wild type E. coli can catabolize both pentose and hexose sugars as carbon sources. In some embodiments of the present disclosure, the recombinant host cell is an E. coli host cell. P. ananatis is also capable of growth under substantially anaerobic conditions; P. ananatis can grow in low pH environments, decreasing the amount of base that must be added during fermentation in order to sustain organic acid (e.g., aspartic acid) production. In some embodiments, the recombinant host cell is a P. ananatis host cell. Vibrio natriegens is one of the fastest growing microbes with a doubling time of under 10 minutes and is suitable as a production host. In some embodiments, the recombinant host cell is a Vibrio natriegens host cell.

### 2.2 Enzymes of the Aspartic Acid Pathway and the β-Alanine Pathway

Provided herein in certain embodiments are recombinant host cells having at least one active aspartic acid pathway from a glycolytic intermediate or glycolytic product to aspartic acid, and/or at least one active β-alanine pathway from aspartic acid to β-alanine. Recombinant host cells having an active aspartic acid pathway and/or β-alanine pathway as used herein produce one or more active enzymes necessary to catalyze each metabolic reaction in an aspartic acid pathway and/or a β-alanine pathway, and therefore are capable of producing aspartic acid and/or β-alanine in measurable yields, titers, and/or productivities when cultured under suitable conditions. Recombinant host cells having an aspartic acid pathway and/or a β-alanine pathway comprise one or more heterologous nucleic acids encoding aspartic acid pathway enzyme(s) and/or β-alanine pathway enzyme(s) and are capable of producing aspartic acid and/or β-alanine.

Recombinant host cells may employ combinations of metabolic reactions for biosynthetically producing the compounds of the present disclosure. The biosynthesized compounds produced by the recombinant host cells include aspartate, aspartic acid, β-alanine, and the intermediates, products and/or derivatives of the aspartic acid pathway and the β-alanine pathway. The biosynthesized compounds can be produced intracellularly and/or secreted into the fermentation medium.

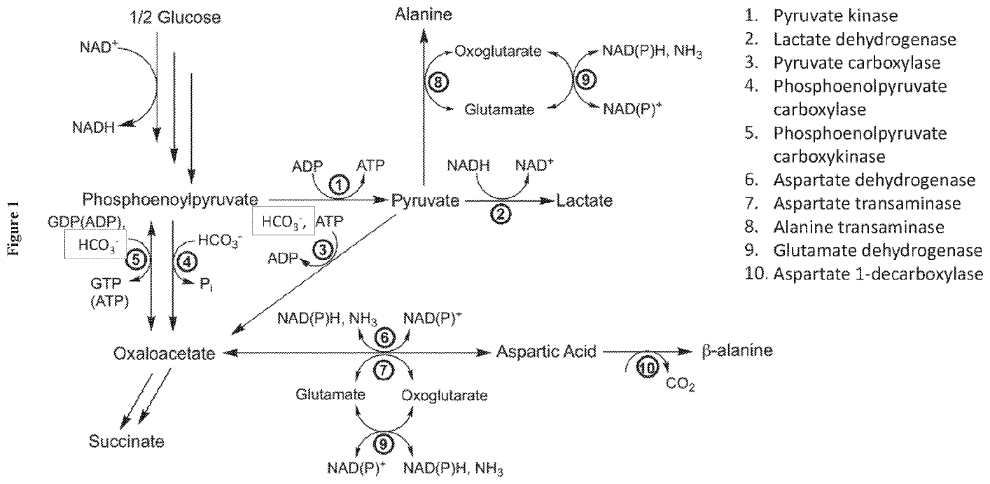

Two enzymatic steps are required to produce aspartate from a glycolytic intermediate or glycolytic product (FIG. 1). The first step uses an oxaloacetate-forming enzyme to convert either phosphoenolpyruvate (a glycolytic intermediate) or pyruvate (a glycolytic product) to oxaloacetate. The second step uses an aspartate-forming enzyme to convert oxaloacetate to aspartate. Both steps take place in the cytosol. The aspartic acid pathways described herein produce two molecules of aspartate from one molecule of glucose.

Enzymes that may function in an aspartic acid pathway are listed in Table 1. In certain embodiments, recombinant host cells comprise one or more heterologous nucleic acids encoding one, two, three, four, five, six, or all seven of the aspartic acid pathway enzymes, or any combination thereof, wherein the heterologous nucleic acids are expressed in sufficient amounts to produce aspartate. In various embodiments, recombinant host cells may comprise multiple copies of a single heterologous nucleic acid and/or multiple copies of two or more heterologous nucleic acids. Recombinant host cells comprising multiple heterologous nucleic acids may comprise any number of heterologous nucleic acids.

An extra enzymatic step is required to convert aspartate to β-alanine (Table 1). This step uses aspartate 1-decarboxylase to convert aspartate to β-alanine and CO₂. The β-alanine pathway described herein produces one molecule of β-alanine from on molecule of aspartate, or two molecules of β-alanine from one molecule of glucose.

TABLE 1
ENZYMES THAT MAY FUNCTION IN AN ASPARTIC
ACID PATHWAY AND/OR A β-ALANINE PATHWAY

| EC # | Enzyme name | Reaction catalyzed |
|---|---|---|
| 6.4.1.1 | Pyruvate carboxylase | Pyruvate + ATP + HCO₃⁻ → ADP + |
| | | Oxaloacetate + Phosphate |
| 4.1.1.31 | Phosphoenolpyruvate carboxylase | Phosphoenolpyruvate + HCO₃⁻ → |
| | | Oxaloacetate + Phosphate |
| 4.1.1.38 | Diphosphate-forming | Phosphoenolpyruvate + Phosphate + |
| | phosphoenolpyruvate | HCO₃⁻ → Oxaloacetate + Diphosphate |
| | carboxykinase | |
| 4.1.1.32 | GTP-forming phosphoenolpyruvate | Phosphoenolpyruvate + GDP + HCO₃⁻ → |
| | carboxykinase | Oxaloacetate + GTP |
| 4.1.1.49 | ATP-forming phosphoenolpyruvate | Phosphoenolpyruvate + ADP + HCO₃⁻ → |
| | carboxykinase | Oxaloacetate + ATP |
| 1.4.1.21 | Aspartate dehydrogenase | Oxaloacetate + NAD(P)H + NH₃ + H⁺ → |
| | | Aspartate + H₂O + NAD(P)⁺ |
| 2.6.1.1 | Aspartate transaminase | Oxaloacetate + Glutamate → Aspartate + |
| | | 2-Oxoglutarate |
| 4.1.1.11 | Aspartate 1-decarboxylase | Aspartate → β-alanine + CO₂ |

In certain embodiments of the present disclosure, the recombinant host cells express some or all of the aspartic acid pathway enzymes, and/or some or all of the β-alanine pathway enzymes, in sufficient amounts to produce aspartic acid and/or β-alanine under substantially anaerobic conditions. Under substantially anaerobic conditions, native aerobic metabolic pathways in recombinant host cells that function to oxidize NAD(P)H are down-regulated. Thus, NAD(P)H is diverted from particular oxygen-dependent pathways to the heterologous aspartic acid pathway for oxidation of NAD(P)H to NAD(P)⁺, providing the driving force for the recombinant host cells to utilize and possibly upregulate the heterologous aspartic acid pathway for redox balance housekeeping. In some embodiments, recombinant host cell native proteins that function to oxidize NAD(P)H may be genetically disrupted to further encourage NAD(P)H oxidization to occur via the heterologous aspartic acid pathway.

The present disclosure also provides consensus sequences (defined above) useful in identifying and/or constructing the aspartic acid pathway and/or β-alanine pathway suitable for use in accordance with the methods of the present disclosure. In various embodiments, these consensus sequences comprise active site amino acid residues believed to be necessary (although the invention is not to be limited by any theory of mechanism of action) for substrate recognition and reaction catalysis, as described below. Thus, an enzyme encompassed by a consensus sequence provided herein has an enzymatic activity that is identical, or essentially identical, or at least substantially similar with respect to ability to catalyze the reaction performed by one of the enzymes exemplified herein. For example, a pyruvate carboxylase as described herein can be used in a host cell of the present disclosure despite comprising insufficient sequence identity with the pyruvate carboxylase consensus sequence.

The construction of recombinant host cells comprising an aspartic acid pathway of the present disclosure is described below in Example 6. Anaerobic fermentation for aspartic acid production and analysis of aspartic acid titers and yields of these recombinant host cells are described below in Example 7.

### 2.2.1 Oxaloacetate-Forming Enzymes

The first step of the aspartic acid pathway comprises converting a glycolytic intermediate or product to oxaloacetate. In various embodiments of the present disclosure, recombinant host cells comprise one or more heterologous nucleic acids encoding an oxaloacetate-forming enzyme wherein the oxaloacetate-forming enzyme is pyruvate carboxylase (EC #6.4.1.1), phosphoenolpyruvate carboxylase (EC #4.1.1.31), GTP-forming phosphoenolpyruvate carboxykinase (EC #4.1.1.32), and/or ATP-forming phosphoenolpyruvate carboxykinase (EC #4.1.1.49), wherein said recombinant host cells are capable of producing aspartic acid. In some embodiments, the recombinant host cells comprise one or more heterologous nucleic acids encoding one, two, three, or all four of the aforementioned oxaloacetate-forming enzymes (FIG. 1 and Table 1). In many embodiments, the oxaloacetate-forming enzyme is derived from a prokaryotic source. In other embodiments, the oxaloacetate-forming enzyme is derived from a eukaryotic source.

### 2.2.1.1 Pyruvate Carboxylase

The pyruvate carboxylase (PYC) (EC #6.4.1.1) described herein catalyzes the conversion of one molecule of pyruvate, one molecule of bicarbonate (HCO₃⁻) and one molecule of ATP to one molecule of oxaloacetate and one molecule of ADP (FIG. 1 and Table 1). Any enzyme is suitable for use in accordance with the invention so long as the enzyme is capable of catalyzing said PYC reaction.

In many embodiments, the PYC is derived from a bacterial source. In many of these embodiments, the PYC is derived from a host cell belonging to a genus selected from the group comprising Corynebacterium, Geobacillus, Rhizobium, Pseudomonas, Mycobacterium, Staphylococcus, Arthrobacter, Sinorhizobium and Methanocaldococcus. Non-limiting examples of bacterial PYC comprise Corynebacterium glutamicum UniProt ID: 054587, Geobacillus thermodenitrificans UniProt ID: A4ILW8, Geobacillus thermodenitrificans UniProt ID: Q05FZ3, Geobacillus stearothermophilus UniProt ID: P94448, Geobacillus stearothermophilus UniProt ID: Q8L1N9, Rhizobium etli UniProt ID: Q2K340, Pseudomonas fluorescence UniProt ID: C3KEC5, Pseudomonas fluorescence UniProt ID: E2XMN3, Pseudomonas fluorescence UniProt ID: V7E6C6, Pseudomonas fluorescence UniProt ID: KOWNR6, Pseudomonas fluorescence UniProt ID: L7HKS9, Pseudomonas fluorescence UniProt ID: J2Y9J8, Pseudomonas fluorescence UniProt ID: U1TDW3, Pseudomonas fluorescence UniProt ID: I4K2J5, Pseudomonas fluorescence UniProt ID: G8QB75, Methanocaldococcus jannaschii UniProt ID: Q58626 and Q58628, Mycobacterium smegmatis UniProt ID: L8FHY2, Mycobacterium smegmatis UniProt ID: I7G857, Mycobacterium smegmatis UniProt ID: I7FNQ9, Mycobacterium smegmatis UniProt ID: AOR6R9, Mycobacterium smegmatis UniProt ID: L8FKA4, Mycobacterium smegmatis UniProt ID: L8FB92, Mycobacterium smegmatis UniProt ID: Q9F843, Mycobacterium smegmatis UniProt ID: A0QV14, and Mycobacterium smegmatis UniProt ID: L8FBY1.

In many embodiments, the PYC is derived from a eukaryotic source. In many of these embodiments, the PYC is derived from a host cell belonging to a genus selected from the group comprising Aspergillus, Paecilomyces, Pichia, Saccharomyces, Phycomyces, Emiliania. Non-limiting examples of eukaryotic PYC comprise Aspergillus niger UniProt ID: Q9HES8, Aspergillus terreus UniProt ID: O93918, Aspergillus oryzae UniProt ID: Q2UGL1, Paecilomyces variotii UniProt ID: V5FWI7, Pichia kudriavzevii UniProt ID: A0A099P757, Pichia kudriavzevii UniProt ID: A0A1V2LT98, Pichia kudriavzevii UniProt ID: A0A1Z8JRB6, Saccharomyces cerevisiae UniProt ID: P11154, Saccharomyces cerevisiae UniProt ID: P32327, Phycomyces blakesleeanus UniProt ID: A0A167KQN5, Phycomyces blakesleeanus UniProt ID: A0A167L0T9, Emiliania huxleyi UniProt ID: B9X0T8.

In some embodiments, the PYC is the C. glutamicum PYC (abbe. CgPYC; UniProt ID: 054587; SEQ ID NO: 15).

In many embodiments, recombinant host cells comprise one or more heterologous nucleic acids encoding a PYC wherein said recombinant host cells are capable of producing aspartic acid and/or β-alanine. In various embodiments, proteins suitable for use in accordance with methods of the present disclosure have PYC activity and comprise an amino acid sequence with at least 40%, at least 50%, at least 60%, at least 70%, at least 80%, at least 90%, or at least 95% sequence identity with SEQ ID NO: 15. In many embodiments, the recombinant host cell is a C. glutamicum strain.

### 2.2.1.2 Phosphoenolpyruvate Carboxylase

The phosphoenolpyruvate carboxylase (PPC) (EC #4.1.1.31) described herein catalyzes the conversion of one molecule of phosphoenolpyruvate and one molecule of HCO₃ to one molecule of oxaloacetate (FIG. 1 and Table 1). The PPC reaction allows for the generation of oxaloacetate from phosphoenolpyruvate instead of pyruvate, circumventing diversion of carbon flux from the aspartic acid pathway to pyruvate, acetyl-CoA, and other central carbon metabolism intermediates which are used by the cell in a variety of reactions. Any enzyme is suitable for use in accordance with the invention so long as the enzyme is capable of catalyzing said PPC reaction.

In many embodiments, the PPC is derived from a prokaryotic source. In many of these embodiments, the PPC is derived from a host cell belonging to a genus selected from the group comprising Acetobacter, Bacillus, Bradyrhizoibum, Brevibacterium, Chlamydomonas, Clostridium, Escherichia, Mycobacterium, Hyphomicrobium, Methanothermobacter, Methanothermus, Photobacterium, Pseudomonas, Rhodospeudomonas, Roseobacter, Starkeya, Streptomyces, Thermosynechococcus, Thiobacillus, Halothiobacillus, Thermus, and Corynebacterium. Non-limiting examples of bacterial PPC comprise Clostridium perfingens UniProt ID: Q8XLE8, Escherichia coli UniProt ID: P00864, Mycobacterium tuberculosis UniProt ID: P9WIH3, Corynebacterium glutamicum UniProt ID: P12880, and Thermosynechococcus vulcanus UniProt ID: P0A3X6.

In many embodiments, the PPC is derived from a eukaryotic source. In many of these embodiments, the PPC is derived from a host cell belonging to a genus selected from the group comprising Alternanthera, Amaranthus, Ananas, Annona, Arabidopsis, Atriplex, Beta, Brachiaria, Brassica, Bryophyllum, Candida, Cicer, Citrus, Coccochloris, Coleataenia, Commelina, Crassula, Cucumis, Digitaria, Echinochloa, Embryophyta, Euglena, Flaveria, Gallus, Glycine, Hakea, Haloxylon, Helianthus, Hordeum, Hydrilla, Iris, Kalanchoe, Lilium, Lotus, Lupinus, Malus, Medicago, Megathyrus, Mesembryanthemum, Molinema, Monoraphidium, Musa, Nicotiana, Oryza, Panicum, Persea, Phaeodactylum, Pichia, Pinus, Pisum, Plasmodium, Portulaca, Ricinus, Saccharomyces, Solanum, Sorghum, Spinacia, Steinchisma, Starkeya, Umbilicus, Vicia, Xylosalsola, and Zea. In some embodiments, the PPC is derived from a fungal source. Non-limiting examples of eukaryotic PPC comprise Alternanthera ficoidea UniProt ID: Q1XAT8, Arabidopsis thaliana UniProt ID: Q5GM68, Arabidopsis thaliana UniProt ID: Q84VW9, Arabidopsis thaliana UniProt ID: Q8GVE8, Arabidopsis thaliana UniProt ID: Q9MAH0, Gossypium hirsutum UniProt ID: 023946, and Pinus halepensis UniProt ID: Q9M3Y3.

In some embodiments, the PPC is the Escherichia coli PPC (abbv. EcPPC; UniProt ID: P00864; SEQ ID NO: 12). In some embodiments, the PPC is the Mycobacterium tuberculosis PPC (abbv. MtPCKG; UniProt ID: P9WIH3; SEQ ID NO: 13). In some embodiments, the PPC is the Corynebacterium glutamicum PPC (abbv. CgPPC; UniProt ID: P12880; SEQ ID NO: 14).

In many embodiments, recombinant host cells comprise one or more heterologous nucleic acids encoding a PPC wherein said recombinant host cells are capable of producing aspartic acid and/or β-alanine. In various embodiments, proteins suitable for use in accordance with methods of the present disclosure have PPC activity and comprise an amino acid sequence with at least 40%, at least 50%, at least 60%, at least 70%, at least 80%, at least 90%, or at least 95% sequence identity with SEQ ID NO: 12, SEQ ID NO: 13, or SEQ ID NO: 14. In many embodiments, the recombinant host cell is a C. glutamicum strain.

In some embodiments, recombinant host cells comprise one or more heterologous nucleic acids encoding a PPC wherein the PPC was mutagenized towards an altered enzyme characteristic such as altered substrate affinity, cofactor affinity, altered reaction rate, and/or altered inhibitor affinity. In these embodiments, the PPC variant is a product of one or more protein engineering cycles. In these embodiments, the PPC variant comprises one or more point mutations. In these embodiments, proteins suitable for use in accordance with methods of the present disclosure have PPC activity and comprise an amino acid sequence with at least 40%, at least 50%, at least 60%, at least 70%, at least 80%, at least 90%, or at least 95% sequence identity with SEQ ID NO: 12, SEQ ID NO: 13, or SEQ ID NO: 14. In some of these embodiments, the PPC variant has decreased affinity for allosteric inhibitors. Non-limiting examples of allosteric inhibitors of PPC include aspartate, acetyl-CoA, and malate. For example, in EcPPC (SEQ ID NO: 12), the allosteric binding site for aspartate is located 20 angstroms away from the catalytic site and the four residues involved in binding aspartate are Lys773, Arg832, Arg587, and Asn881. In some embodiments, proteins with at least 40% sequence identity with SEQ ID NO: 12 comprise a mutation at one, some, or all of these amino acids to decrease binding of aspartate. In embodiments wherein the recombinant host cells comprise one or more heterologous nucleic acids encoding such a mutagenized PPC, the recombinant host cells produce aspartate at a titer and/or yield that is higher than recombinant host cells lacking said mutagenized PPC.

The PPC consensus sequence #1 (SEQ ID NO: 35) provides the sequence of amino acids in which each position identifies the amino acid (if a specific amino acid is identified) or a subset of amino acids (if a position is identified as variable) most likely to be found at a specific position in a PPC. Many amino acids in consensus sequence #1 (SEQ ID NO: 35) are highly conserved and PPCs suitable for use in accordance with the methods of the present disclosure will comprise a substantial number, and sometimes all, of these highly conserved amino acids at positions aligning with the location of the indicated amino acids in consensus sequence #1 (SEQ ID NO: 35). In various embodiments, proteins suitable for use in accordance with the methods of the present disclosure have PPC activity and comprise an amino acid sequence with at least 40%, at least 50%, at least 60%, at least 65%, or at least 70% sequence identity with consensus sequence #1 (SEQ ID NO: 35). For example, the EcPPC sequence (SEQ ID NO: 12 is at least 40% identical to consensus sequence #1 (SEQ ID NO: 35) and is therefore encompassed by consensus sequence #1 (SEQ ID NO: 35).

In enzymes homologous to SEQ ID NO: 35, amino acids that are highly conserved are Ml, Y5, N11, S13, M14, L15, G16, L19, G20, T22, 123, A26, G28, E36, 138, R39, L41, S42, R46, G48, R53, L56, P70, V71, A72, R73, A74, F75, Q77, F78, L79, N80, L81, N83, A85, E86, Q87, Y88, 191, S92, L111, V125, E131, L132, V133, L134, T135, A136, H137, P138, T139, E140, R143, R144, K149, N154, C156, L157, L160, E169, L177, L180, A182, W185, H186, I190, R191, R194, P195, P197, E200, A201, K202, W203, G204, A206, E209, N210, S211, L212, W213, P217, L220, R221, L235, P241, W247, M248, G249, G250, D251, R252, D253, G254, N255, P256, V258, T259, T263, R271, W272, K273, A274, L277, L279, D281, L285, E288, L289, S290, G303, E309, P310, Y311, R312, K316, R319, L322, T325, L351, W352, P354, L355, C358, Y359, S361, L362, C365, G366, M367, 1369, 1370, A371, G373, L375, L376, D377, L379, R381, F385, G386, L389, D393, R395, Q396, E397, S398, T399, H₄₀₁, E407, Y411, G415, D416, Y417, W420, E422, K425, F428, L429, E432, L433, S435, R437, P438, L439, P441, W444, P446, S447, E452, T456, C457, Y471, 1473, S474, M475, A476, S480, D481, V482, L483, A484, V485, L487, L488, L489, E491, G493, V500, P502, L503, F504, E505, T506, L507, D509, L510, L520, W525, Y526, R527, 1530, Q534, M535, V536, M537, 1538, G539, Y540, S541, D542, S543, A544, K545, D546, A547, G548, M550, A552, W554, A555, Q556, Y557, A559, L563, L574, T575, L576, F577, H578, G579, R580, G581, G582, 1584, G585, R586, G587, G588, A589, P590, A591, H592, A594, L595, L596, S597, Q598, P599, P600, S602, L603, K604, G606, L607, R608, V609, T610, E611, Q612, G613, E614, M615, 1616, R617, F618, K619, G621, L622, P623, Y633, A636, L638, E639, A640, N641, L642, L643, P644, P645, P646, P648, K649, W652, M656, L659, S660, S663, C664, Y667, R668, R672, F677, V678, Y680, F681, R682, A684, T685, P686, E687, E689, L690, K692, L693, P694, L695, G696, S697, R698, P699, A700, K701, R702, P704, G706, G707, V708, E709, L711, R712, A713, 1714, P715, W716, 1717, F718, W720, Q722, N723, R724, L725, L727, P728, A729, W730, L731, G732, A733, G734, G744, M752, W756, P757, F758, F759, T761, R762, M765, L766, E767, M768, V769, K772, Y781, D782, L785, L790, W791, L793, G794, L797, R798, D804, 1805, V808, L809, L817, M818, P822, W823, 1828, L830, R831, N832, Y834, P837, L838, N839, L841, Q842, E844, L845, L846, R848, R850, E860, A862, L863, M864, 1867, G869, A871, G873, M874, R875, N876, T877, and G878. In various embodiments, PPC enzymes homologous to SEQ ID NO: 35 comprise at least 40%, at least 50%, at least 60%, at least 70%, at least 80%, at least 85%, at least 90%, at least 95%, or sometimes all of these highly conserved amino acids at positions corresponding to the highly conserved amino acids identified in SEQ ID NO: 35. In some embodiments, each of these highly conserved amino acids are found in a desired PPCs as provided, for example, in SEQ ID NO: 12.

### 2.2.1.3 Phosphoenolpyruvate Carboxykinase

The phosphoenolpyruvate carboxykinases described herein catalyzes the conversion of one molecule of phosphoenolpyruvate and one molecule of HCO₃⁻ to one molecule of oxaloacetate (FIG. 1 and Table 1). Similar to the PPC, the phosphoenolpyruvate carboxykinase (PCK) reaction allows for the generation of oxaloacetate from phosphoenolpyruvate instead of pyruvate, circumventing diversion of carbon flux from the aspartic acid pathway to pyruvate, acetyl-CoA, and other central carbon metabolism intermediates which are used by the cell in a variety of reactions. Any enzyme is suitable for use in accordance with the invention so long as the enzyme is capable of catalyzing said PCK reaction. PCK comes in three types (Table 1): Diphosphate-forming PCK (EC #4.1.1.38), GTP-forming PCK (EC #4.1.1.32), and ATP-forming PCK (EC #4.1.1.49). Diphosphate-forming PCK (EC #4.1.1.38) converts one molecule of phosphoenolpyruvate, one molecule of CO₂ and one molecule of phosphate to one molecule of oxaloacetate and one molecule of diphosphate. GTP-forming PCK (EC #4.1.1.32) converts one molecule of phosphoenolpyruvate, one molecule of CO₂ and one molecule of GDP to one molecule of oxaloacetate and one molecule of GTP. ATP-forming PCK (EC #4.1.1.49) converts one molecule of phosphoenolpyruvate, one molecule of CO₂ and one molecule of ADP to one molecule of oxaloacetate and one molecule of ATP. While all three PCK types are suitable for uses in accordance with the methods of the invention, it is often desirable to use an ATP-forming PCK since ATP is broadly useful by the cell for maintenance of cellular health and vitality. In many embodiments, the PCK is an ATP-forming PCK. In various embodiments, proteins suitable for use in accordance with the methods of the present disclosure have PCK activity and comprise an amino acid sequence with at least 40%, at least 50%, at least 60%, at least 65%, at least 70%, or at least 90% sequence identity with EcPCKA (UniProt ID: P22259; SEQ ID NO: 18).

In many embodiments, the PCK is derived from a bacterial source. In many of these embodiments, the PCK is derived from a host cell belonging to a genus selected from the group comprising Actinobacillus, Escherichia, Anaerobiospirillum, Bacillus, Corynebacterium, Cupriavidus, Leishmania, Rhodopseudomonas, Ruminiclostridium, Ruminococcus, Salinivibrio, Selenomonas, Sinorhizobium, Staphylococcus, Mannheimia, Haemophilus, and Thermus. Non-limiting examples of bacterial PCK comprise Actinobacillus ficoidea UniProt ID: Q6W6X5, Anaerobiospirillum succiniciproducens UniProt ID: 009460, E. coli UniProt ID: P22259, Anaerobiospirillum succiniciproducens UniProt ID: 009460, Actinobacillus succinogenes UniProt ID: A6VKV4, Corynebacterium glutamicum UniProt ID: Q9AEM1, Mannheimia succiniciproducens UniProt ID: Q65Q60, Ruminococcus albus UniProt ID: B3Y6D3, Selenomonas ruminantium UniProt ID: 083023, Thermus thermophiles UniProt ID: Q5SLL5, and Haemophilus influenzae UniProt ID: A5UDR5.

In many embodiments, the PCK is derived from a eukaryotic source. In many of these embodiments, the pyruvate carboxykinase is derived from a host cell belonging to a genus selected from the group comprising Alternanthera, Ananas, Arabidopsis, Candida, Clusia, Cucumis, Digitaria, Embryophyta, Hordeum, Iris, Laminaria, Megathyrus, Mus, Nicotiana, Oryza, Pichia, Pisum, Plasmodium, Prunus, Saccharomyces, Skeletonema, Solanum, Solenostemon, Sorghum, Tillandsia, Trypanosoma, Udotea, Urochloa, Vitis, Pichia, Aspergillus, Zoysia and Zea. In some embodiments, the PCK is derived from a fungal source. Non-limiting examples of eukaryotic PCK comprise Arabidopsis thaliana UniProt ID: Q93VK0, Plasmodium falciparum UniProt ID: Q9U750, Saccharomyces cerevisiae UniProt ID: P10963, Pichia kudriavzevii UniProt ID: A0A099NX43, and Zoysia japonica UniProt ID: Q5KQS7.

In some embodiments, the PCK is the Actinobacillus succinogenes PCK (abbv. AsPCKA; UniProt ID: A6VKV4; SEQ ID NO: 16). In some embodiments, the PCK is the Corynebacterium glutamicum PCK (abbv. CgPCKG; UniProt ID: Q9AEM1; SEQ ID NO: 17). In some embodiments, the PCK is the E. coli PCK (abbv. EcPCKA; UniProt ID: P22259; SEQ ID NO: 18).

In many embodiments, recombinant host cells comprise one or more heterologous nucleic acids encoding a PCK wherein said recombinant host cells are capable of producing aspartic acid and/or β-alanine. In various embodiments, proteins suitable for use in accordance with methods of the present disclosure have pyruvate carboxykinase activity and comprise an amino acid sequence with at least 40%, at least 50%, at least 60%, at least 70%, at least 80%, at least 90%, or at least 95% sequence identity with SEQ ID NO: 16, SEQ ID NO: 17, SEQ ID NO: 18. In many embodiments, the recombinant host cell is a C. glutamicum strain.

### 2.2.2 Aspartate-Forming Enzymes

The second step of the aspartic acid pathway comprises converting oxaloacetate to aspartate. In various embodiments of the present disclosure, recombinant host cells comprise one or more heterologous nucleic acids encoding an aspartate-forming enzyme wherein the aspartate-forming enzyme is aspartate dehydrogenase and/or aspartate transaminase, wherein said recombinant host cells are capable of producing aspartic acid. In some embodiments, the recombinant host cells comprise one or more heterologous nucleic acids encoding one, two, three, or all four of the aforementioned oxaloacetate-forming enzymes (FIG. 1 and Table 1). In many embodiments, the aspartate-forming enzyme is derived from a prokaryotic source. In other embodiments, the aspartate-forming enzyme is derived from a eukaryotic source.

### 2.2.2.1 Aspartate Dehydrogenase

The aspartate dehydrogenase (AspDH) (EC #1.4.1.21) described herein catalyzes the conversion of one molecule of oxaloacetate, one molecule of NAD(P)H, one molecule of NH₃ and one proton to one molecule of aspartate, one molecule of H₂O and one molecule of NAD(P)⁺ (FIG. 1 and Table 1). Any enzyme is suitable for use in accordance with the invention so long as the enzyme is capable of catalyzing said AspDH reaction.

In most cell types, the pool of NAD (which consists of reduced and oxidized forms, i.e., NADH and NAD⁺), is larger than that of NADP (which consists of reduced and oxidized forms, i.e., NADPH and NADP⁺). Under certain fermentation conditions, NADP may be even more scarce. Further, while interconversion of NADP with NAD can occur, the process is slow and inefficient. The limited availability and low regeneration rate of NADPH can hamper enzyme turnover and product titers, yields or productivities during fermentation. Native enzyme cofactor specificity can be altered, however, by standard microbial engineering techniques, and recombinant host cells can be designed to express modified enzymes that utilize NADH, or NADH and NADPH non-selectively, instead of NADPH exclusively.

AspDH is able to utilize either NADH or NADPH as a cofactor. Generally, NADH is produced during the recombinant host cell's glycolytic processes in converting glucose to pyruvate. In C. glutamicum, P. kudriavzevii, S. cerevisiae, P. ananatis, and E. coli, for example, the glyceraldehyde 3-phosphate dehydrogenase (GAPDH) in glycolysis reduces NAD⁺ to NADH; therefore, in embodiments wherein GAPDH produces NADH, the AspDH is NADH-utilizing to ensure AspDH turnover is not impeded as AspDH is able to utilize readily available NADH. Similarly, in other embodiments wherein the AspDH is NADPH-utilizing, it is beneficial to utilize a GAPDH that converts NADP⁺ to NADPH in glycolysis. In Kluyveromyces lactis and Clostridium acetobutylicum, for example, natively expressed GAPDH reduces NADP⁺ to generate NADPH. Thus, in embodiments wherein the GAPDH produces NADPH, the AspDH is NADPH-utilizing. Details on NADPH-producing/NADP⁺-utilizing GAPDH are disclosed below in section 2.5.1.2.

The AspDHs of the present disclosure comprise: (1) NADH-utilizing AspDH; (2) NADPH-utilizing AspDH; and (3) AspDH that can indiscriminately utilize NADH and NADPH. In some embodiments, the recombinant host cells comprise an AspDH that utilizes NADH as a cofactor and is capable of producing aspartic acid and/or β-alanine. In some embodiments, the recombinant host cells comprise an AspDH that utilizes NADPH as a cofactor and is capable of producing aspartic acid and/or β-alanine. In some embodiments, the recombinant host cells comprise an AspDH that utilizes NADH and/or NADPH as a cofactor and is capable of producing aspartic acid and/or β-alanine. In embodiments wherein the AspDH is capable of utilizing NADH and NADPH, recombinant host cells may further comprise a transhydrogenase (EC #1.6.1.1, 1.6.1.2, or 1.6.1.5).

In many embodiments, the AspDH is derived from a prokaryotic source. In many of these embodiments, the AspDH is derived from a host cell belonging to a genus selected from the group comprising Bradyrhizobium, Escherichia, Thermotoga, Klebsiella, Cupriavidus, Rhodopseudomonas, Pseudomonas, Variovorax, Delftia, Ralstonia, Burkholderia, Ochrobactrum, Acinetobacter, Dinoroseobacter, Ruegeria, Herbaspirillum, and Comamonas. Non-limiting examples of prokaryotic AspDH enzymes include the Pseudomonas aeruginosa UniProt ID: Q9HYA4 (abbv. PaAspDH), Cupriavidus taiwanensis UniProt ID: B3R8S4 (abbv. AspDH #2), the Polaromonas sp. UniProt ID: Q126FS (abbv. AspDH #4), Klebsiella pneumoniae UniProt ID: A6TDT8 (abbv. AspDH #9), Comamonas testosteroni UniProt ID: D0IX49 (abbv. AspDH #12), Delftia acidovarans UniProt ID: S2WWY2 (abbv. AspDH #14), Variovorax sp. UniProt ID: A0A1C6Q9L7 (abbv. AspDH #16), Thermotoga maritima UniProt ID: Q9X1X6 (abbv. TmAspDH), Ralstonia solanacearum UniProt ID: Q8XRV9 (abbv. AspDH #3), Burkholderia thailandensis UniProt ID: Q2T559 (abbv. AspDH #5), Burkholderia pseudomallei UniProt ID: Q3JFK2 (abbv. AspDH #6), Ochrobactrum anthropic UniProt ID: A6X792 (abbv. AspDH #7), Acinetobacter sp. UniProt ID: D6JRV1 (abbv. AspDH #8), Dinoroseobacter shibae UniProt ID: A8LLH8 (abbv. AspDH #10), Rugeria pomeroyi UniProt ID: Q5LPG8 (abbv. AspDH #11), Ralstonia eutropha UniProt ID: Q46VA0 (abbv. AspDH #13), Pseudomonase sp. ENNP23 UniProt ID: A0A1E4W5J7 (abbv. AspDH #15), Herbaspirillum frisingense UniProt ID: R0EI78 (abbv. AspDH #17), Burkholderiaceae bacterium 16 UniProt ID: A0A0F0FQG4 (abbv. AspDH #18), Ralstonia sp. GA3-3 UniProt ID: R7XIB1 (abbv. AspDH #19), Cupriavidus sp. SK-3 UniProt ID: A0A069IKY7 (abbv. AspDH #20) and Cupriavidus necator UniProt ID: Q46VA0 (abbv. CnAspDH).

In many embodiments, the AspDH is derived from an archaeal source. In many of these embodiments, the AspDH is derived from a host cell belonging to the genus Archaeoglobus. A non-limiting example of archaeal AspDH is the A. fulgidus UniProt ID: 028440.

In some embodiments, the AspDH is the Cupriavidus taiwanensis AspDH (abbv. AspDH #2; UniProt ID: B3R8S4; SEQ ID NO: 19). In some embodiments, the AspDH is the Polaromonas sp. AspDH (abbv. AspDH #4; UniProt ID: Q126FS; SEQ ID NO: 20). In some embodiments, the AspDH is the Klebsiella pneumoniae AspDH (abbv. AspDH #9; UniProt ID: A6TDT8; SEQ ID NO: 21). In some embodiments, the AspDH is the Comamonas testosteroni AspDH (abbv. AspDH #12; UniProt ID: D0IX49; SEQ ID NO: 24). In some embodiments, the AspDH is the Delftia acidovarans AspDH (abbv. AspDH #14; UniProt ID: S2WWY2; SEQ ID NO: 22). In some embodiments, the AspDH is the Variovorax sp. AspDH (abbv. AspDH #16; UniProt ID: A0A1C6Q9L7; SEQ ID NO: 23). In some embodiments, the AspDH is the Pseudomonase aeruginosa AspDH (abbv. PaAspDH; UniProt ID: Q9HYA4; SEQ ID NO: 34). In some embodiments, the AspDH is the Ralstonia solanacearum UniProt ID: Q8XRV9 (abbv. AspDH #3). In some embodiments, the AspDH is the Burkholderia thailandensis UniProt ID: Q2T559 (abbv. AspDH #5). In some embodiments, the AspDH is the Burkholderia pseudomallei UniProt ID: Q3JFK2 (abbv. AspDH #6). In some embodiments, the AspDH is the Ochrobactrum anthropic UniProt ID: A6X792 (abbv. AspDH #7). In some embodiments, the AspDH is the Acinetobacter sp. UniProt ID: D6JRV1 (abbv. AspDH #8). In some embodiments, the AspDH is the Dinoroseobacter shibae UniProt ID: A8LLH8 (abbv. AspDH #10). In some embodiments, the AspDH is the Rugeria pomeroyi UniProt ID: Q5LPG8 (abbv. AspDH #11). In some embodiments, the AspDH is the Ralstonia eutropha UniProt ID: Q46VA0 (abbv. AspDH #13). In some embodiments, the AspDH is the Pseudomonase sp. ENNP23 UniProt ID: A0A1E4W5J7 (abbv. AspDH #15). In some embodiments, the AspDH is the Herbaspirillum frisingense UniProt ID: R0EI78 (abbv. AspDH #17). In some embodiments, the AspDH is the Burkholderiaceae bacterium 16 UniProt ID: A0A0F0FQG4 (abbv. AspDH #18). In some embodiments, the AspDH is the Ralstonia sp. GA3-3 UniProt ID: R7XIB1 (abbv. AspDH #19). In some embodiments, the AspDH is the Cupriavidus sp. SK-3 UniProt ID: A0A069IKY7 (abbv. AspDH #20)

In many embodiments, recombinant host cells comprise one or more heterologous nucleic acids encoding an AspDH wherein said recombinant host cells are capable of producing aspartic acid and/or β-alanine. In various embodiments, proteins suitable for use in accordance with methods of the present disclosure have AspDH activity and comprise an amino acid sequence with at least 40%, at least 50%, at least 60%, at least 70%, at least 80%, at least 90%, or at least 95% sequence identity with SEQ ID NO: 19, SEQ ID NO: 20, SEQ ID NO: 21, SEQ ID NO: 22, SEQ ID NO: 23, SEQ ID NO: 24, or SEQ ID NO: 34. In various embodiments, proteins suitable for use in accordance with methods of the present disclosure have AspDH activity and comprise an amino acid sequence with at least 40%, at least 50%, at least 60%, at least 70%, at least 80%, at least 90%, or at least 95% sequence identity with AspDH #3, AspDH #5, AspDH #6, AspDH #7, AspDH #8, AspDH #10, AspDH #11, AspDH #13, AspDH #15, AspDH #17, AspDH #18, AspDH #19, or AspDH #20. In many embodiments, the recombinant host cell is a C. glutamicum strain.

In some embodiments, recombinant host cells comprise one or more heterologous nucleic acids encoding an AspDH wherein the AspDH was mutagenized towards an altered enzyme characteristic such as altered substrate affinity, cofactor affinity, altered reaction rate, and/or altered inhibitor affinity. In these embodiments, the AspDH variant is a product of one or more protein engineering cycles. In these embodiments, the AspDH variant comprises one or more point mutations. In these embodiments, proteins suitable for use in accordance with methods of the present disclosure have AspDH activity and comprise an amino acid sequence with at least 40%, at least 50%, at least 60%, at least 70%, at least 80%, at least 90%, or at least 95% sequence identity with SEQ ID NO: 19, SEQ ID NO: 20, SEQ ID NO: 21, SEQ ID NO: 22, SEQ ID NO: 23, SEQ ID NO: 24, or SEQ ID NO: 34. In these embodiments, proteins suitable for use in accordance with the methods of the present disclosure have AspDH activity and comprise an amino acid sequence with at least 40%, at least 50%, at least 60%, at least 70%, at least 80%, at least 90%, or at least 95% sequence identity with AspDH #3, AspDH #5, AspDH #6, AspDH #7, AspDH #8, AspDH #10, AspDH #11, AspDH #13, AspDH #15, AspDH #17, AspDH #18, AspDH #19, or AspDH #20. In some of these embodiments, the AspDH variant has increased affinity for NADH. In many embodiments, the recombinant host cell is a C. glutamicum strain.

The AspDH consensus sequence #2 (SEQ ID NO: 33) provides the sequence of amino acids in which each position identifies the amino acid (if a specific amino acid is identified) or a subset of amino acids (if a position is identified as variable) most likely to be found at a specific position in an AspDH. Many amino acids in consensus sequence #2 (SEQ ID NO: 33) are highly conserved and AspDHs suitable for use in accordance with the methods of the present disclosure will comprise a substantial number, and sometimes all, of these highly conserved amino acids at positions aligning with the location of the indicated amino acids in consensus sequence #2 (SEQ ID NO: 33). In various embodiments, proteins suitable for use in accordance with the methods of the present disclosure have AspDH activity and comprise an amino acid sequence with at least 40%, at least 50%, at least 60%, at least 65%, or at least 70% sequence identity with consensus sequence #2 (SEQ ID NO: 33). For example, the PaAspDH sequence (SEQ ID NO: 34) is at least 40% identical to consensus sequence #2 (SEQ ID NO: 33), and is therefore encompassed by consensus sequence #2 (SEQ ID NO: 33).

In enzymes homologous to SEQ ID NO: 33, amino acids that are highly conserved are G8, G10, All, 112, G13, E69, A71, G72, H73, A75, H79, P82, L84, G87, S94, G96, A97, L98, A110, A111, G114, L120, G123, A124, 1125, G126, D129, A130, A133, A134, G137, G138, L139, V142, Y144, G146, R147, K148, P149, W153, T156, P157, E159, D163, L164, 1173, F174, G176, A178, A181, A182, P186, K187, N188, A189, N190, V191, A192, A193, T194, A198, G199, G201, L202, T205, V207, L209, A211, D212, P213, N218, H220, A224, G226, A227, F228, G229, L233, P239, L240, N243, P244, K245, T246, S247, A248, L249, T250, S253, R256, A257, N260, and 1267. In various embodiments, AspDH enzymes homologous to SEQ ID NO: 33 comprise at least 40%, at least 50%, at least 60%, at least 70%, at least 80%, at least 85%, at least 90%, at least 95%, or sometimes all of these highly conserved amino acids at positions corresponding to the highly conserved amino acids identified in SEQ ID NO: 33. In some embodiments, each of these highly conserved amino acids are found in a desired AspDHs as provided, for example, in SEQ ID NO: 19, SEQ ID NO: 20, SEQ ID NO: 21, SEQ ID NO: 22, SEQ ID NO: 23, SEQ ID NO: 24, and SEQ ID NO: 34.

Amino acid H220 in SEQ ID NO: 33 functions as a general acid/base (although the invention is not to be limited by any theory of mechanism of action) and is necessary for enzyme activity; thus, an amino acid corresponding to H220 in consensus sequence SEQ ID NO: 33 is found in enzymes homologous to SEQ ID NO: 33. For example, the strictly conserved amino acid corresponding to H220 in consensus sequence SEQ ID NO: 33 is found in AspDHs set forth in SEQ ID NO: 19, SEQ ID NO: 20, SEQ ID NO: 23, and SEQ ID NO: 34.

### 2.2.2.2 Aspartate Transaminase

The aspartate transaminase (AspB) (EC #2.6.1.1) described herein catalyzes the conversion of one molecule of oxaloacetate and one molecule of glutamate to one molecule of aspartate and one molecule of 2-oxoglutarate (which is synonymous with oxoglutarate) (FIG. 1 and Table 1). Any enzyme is suitable for use in accordance with the invention so long as the enzyme is capable of catalyzing said AspB reaction.

In many embodiments, the AspB is derived from a prokaryotic source. In many of these embodiments, the AspB is derived from a host cell belonging to a genus selected from the group comprising Bacillus, Corynebacterium, Escherichia, Mycobacterium, Deinococcus, Giardia, Leishmania, Leptosphaeria, Sinorhizobium, and Nostoc. Non-limiting examples of prokaryotic AspB include Corynebacterium glutamicum UniProt ID: Q8NTR2 (abbv. CgAspB), Corynebacterium diphtheriae UniProt ID: Q6NJY4 (abbv. CdAspB), Deinococcus geothermalis UniProt ID: Q1IZU0 (abbv. DgAspB), Mycobacterium tuberculosis UniProt ID: 069689 (abbv. MtAspB).

In many embodiments, the AspB is derived from a eukaryotic source. In many of these embodiments, the AspB is derived from a host cell belonging to a genus selected from the group comprising Arabidopsis, Crassostrea, Sulfolobus, Trypanosoma and Xenopus.

In some embodiments, the AspB is the C. glutamicum AspB (abbv. CgAspB; UniProt ID: Q8NTR2; SEQ ID NO: 25). In some embodiments, the AspB is the C. diphtheriae AspB (abbv. CdAspB; UniProt IDQ6NJY4; SEQ ID NO: 26). In some embodiments, the AspB is the D. geothermalis AspB (abbv. DgAspB; UniProt ID: DIP0257; SEQ ID NO: 27). In some embodiments, the AspB is the M. tuberculosis AspB (abbv. MtAspB; UniProt ID: 069689; SEQ ID NO: 28)

In many embodiments, recombinant host cells comprise one or more heterologous nucleic acids encoding an AspB wherein said recombinant host cells are capable of producing aspartic acid and/or β-alanine. In various embodiments, proteins suitable for use in accordance with methods of the present disclosure have AspB activity and comprise an amino acid sequence with at least 40%, at least 50%, at least 60%, at least 70%, at least 80%, at least 90%, or at least 95% sequence identity with SEQ ID NO: 25, SEQ ID NO: 26, SEQ ID NO: 27, or SEQ ID NO: 28. In many embodiments, the recombinant host cell is a C. glutamicum strain.

The AspB consensus sequence #3 (SEQ ID NO: 36) provides the sequence of amino acids in which each position identifies the amino acid (if a specific amino acid is identified) or a subset of amino acids (if a position is identified as variable) most likely to be found at a specific position in an AspB derived from Corynebacterium and related prokaryotes. Many amino acids in consensus sequence #3 (SEQ ID NO: 36) are highly conserved and AspBs suitable for use in accordance with the methods of the present disclosure will comprise a substantial number, and sometimes all, of these highly conserved amino acids at positions aligning with the location of the indicated amino acids in consensus sequence #3 (SEQ ID NO: 36). In various embodiments, proteins suitable for use in accordance with the methods of the present disclosure have AspB activity and comprise an amino acid sequence with at least 40%, at least 50%, at least 60%, at least 65%, or at least 70% sequence identity with consensus sequence #3 (SEQ ID NO: 36). For example, the CgAspB sequence (SEQ ID NO: 25) is at least 40% identical to consensus sequence #3 (SEQ ID NO: 36), and is therefore encompassed by consensus sequence #3 (SEQ ID NO: 36).

In enzymes derived from Corynebacterium and related prokaryotes that are homologous to SEQ ID NO: 36, amino acids that are highly conserved are L25, L30, L32, L34, T35, R36, G37, K38, P39, E42, Q43, L44, D45, L50, L51, L53, P54, G64, D66, R68, N69, Y70, G71, G75, R80, A96, S101, L102, D107, G116, D119, S120, P123, W124, E127, K131, C134, P135, P137, G138, Y139, D140, R141, H142, 1145, G150, E152, M153, P157, G162, P163, D164, L171, V172, P176, K179, G180, W182, V184, P185, N189, P190, T191, G192, M206, A209, A210, P211, D212, F213, R214, W217, D218, N219, A220, Y221, V223, L226, A243, and G244.

In many embodiments wherein recombinant host cells comprise one or more heterologous nucleic acids encoding an AspB and said recombinant host cells are capable of producing aspartic acid and/or β-alanine, said recombinant host cells may further comprise heterologous nucleic acids encoding a glutamate dehydrogenase (EC #1.4.1.2 or 1.4.1.3). The oxoglutarate produced by AspB (with concomitant production of aspartate) needs to be converted back to glutamate for future aspartate transaminase reactions. In various embodiments, proteins suitable for use in accordance with methods of the present disclosure have glutamate dehydrogenase activity. Details on glutamate dehydrogenase are disclosed below in section 2.5.1.1

Aspartate 1-Decarboxylase

In the β-alanine pathway of the present disclosure, β-alanine is produced via decarboxylation of aspartate. The aspartate 1-decarboxylase (PanD) (EC #4.1.1.11) described herein catalyzes the conversion of one molecule of aspartate to one molecule of β-alanine and one molecule of CO₂ (FIG. 1 and Table 1). Thus, in many embodiments wherein recombinant host cells are capable of producing β-alanine, the recombinant host cells comprise heterologous nucleic acids encoding aspartic acid enzymes and PanD. Any enzyme is suitable for use in accordance with the invention so long as the enzyme is capable of catalyzing said PanD reaction.

Proteins capable of catalyzing said PanD reaction provided herein include both bacterial and eukaryotic types. Bacterial PanDs are pyruvoyl-dependent decarboxylases where the covalently bound pyruvoyl cofactor is produced by autocatalytic rearrangement of a specific serine residues (e.g., S25 in SEQ IDs NO: 29 and 37). Eukaryotic PanDs, in contrast, do not possess a pyruvoyl cofactor and instead possess a pyridoxal 5′-phosphate cofactor. In some embodiments, the recombinant host cell comprises a heterologous nucleic acid encoding a bacterial PanD and is capable of producing β-alanine. In other embodiments, the recombinant host cell comprises a heterologous nucleic acid encoding a eukaryotic PanD and is capable of producing β-alanine.

In many embodiments, the PanD is derived from a bacterial source. In many of these embodiments, the PanD is derived from a host cell belonging to a genus selected from the group comprising Corynebacterium, Escherichia, Helicobacter, Methanocaldococcus, Mycobacterium, Bacillus, Clostridium, Enterococcus, Lactobacillus, Cupriavidus, Arthrobacter, Pseudomonas, Staphylococcus, Streptomyces, and Salmonella. Non-limiting examples of bacterial PanD include Corynebacterium glutamicum UniProt ID: Q9X4N0 (abbv. CgAPanD), Escherichia coli UniProt ID: P0A790, and Methanocaldococcus jannaschii UniProt ID: Q60358, Arthrobacter aurescens UniProt ID: A1RDH3, Bacillus cereus UniProt ID: A7GN78, Bacillus subtilis UniProt ID: P52999, Burkholderia xenovorans UniProt ID: Q143J3, Clostridium acetobutylicum UniProt ID: P58285, Clostridium beijerinckii UniProt ID: A6LWN4, Corynebacterium efficiens UniProt ID: Q8FU86, Corynebacterium jeikeium UniProt ID: Q4JXL3, Cupriavidus necator UniProt ID: Q9ZHI5, Enterococcus faecalis UniProt ID: Q833S7, E. coli UniProt ID: Q0TLK2, Helicobacter pylori UniProt ID: P56065, Lactobacillus plantarum UniProt ID: Q88Z02, Mycobacterium smegmatis UniProt ID: A0QNF3, Pseudomonas aeruginosa UniProt ID: Q9HV68, Pseudomonas fluorescens UniProt ID: Q848I5, Staphylococcus aureus UniProt ID: A6U4X7, and Streptomyces coelicolor UniProt ID: P58286.

In many embodiments, the PanD is derived from a eukaryotic source. In many of these embodiments, the PanD is derived from a host cell belonging to a genus selected from the group comprising Aedes, Drosophila, and Tribolium. Non-limiting examples of eukaryotic PanD include Tribolium castaneum UniProt ID: A7U8C7, Tribolium castaneum UniProt ID: A9YVA8, Aedes aegypti UniProt ID: Q171S0, Drosophila mojavensis UniProt ID: B4KIX9, and Dendroctonus ponderosas UniProt ID: U4UTD4.

In some embodiments, the PanD is the C. glutamicum PanD (abbv. CgPanD; UniProt ID: Q9X4N0; SEQ ID NO: 29). In some embodiments, the PanD is the B. subtilis PanD (abbv. BsPanD; UniProt ID: P52999; SEQ ID NO: 37). In some embodiments, the PanD is the T. castaneum PanD (abbv. TcPanD; UniProt ID: A9YVA8; SEQ ID NO: 38).

In many embodiments, recombinant host cells comprise one or more heterologous nucleic acids encoding a PanD wherein said recombinant host cells are capable of producing β-alanine. In various embodiments, proteins suitable for use in accordance with methods of the present disclosure have PanD activity and comprise an amino acid sequence with at least 40%, at least 50%, at least 60%, at least 70%, at least 80%, at least 90%, or at least 95% sequence identity with SEQ ID NO: 29, SEQ ID NO: 37, or SEQ ID NO: 38. In many embodiments, the recombinant host cell is a C. glutamicum strain.

A number of amino acids in both bacterial and eukaryotic PanDs provided herein are highly conserved, and proteins homologous to either a bacterial or a eukaryotic PanD of the present disclosure may comprise amino acids corresponding to a substantial number of highly conserved amino acids. As described above, a homolog is said to comprise a substantial number of amino acids corresponding to highly conserved amino acids in a reference sequence if at least 40%, at least 50%, at least 60%, at least 70%, at least 80%, at least 90%, at least 95%, or more than 95% of the highly conserved amino acids in the reference sequence are found in the homologous protein.

In some embodiments, the PanD comprises a bacterial PanD, such as CgPanD (SEQ ID NO: 29), BsPanD (SEQ ID NO: 37), or other bacterial PanD. The highly conserved amino acids in bacterial PanD are K9, H11, R12, A13, V15, T16, A18, L20, Y22, G24, S25, D29, E42, N51, G52, R54, T57, Y58, 160, G62, G65, G67, N72, G73, A74, A75, A76, G82, D83, V85, I86, Y90, E97, P103, and N112. In some embodiments, proteins homologous to CgPanD (SEQ ID NO: 29) comprise amino acids corresponding to at least 40%, at least 50%, at least 60%, at least 70%, at least 80%, at least 85%, at least 90%, at least 95%, or more than 95% of these highly conserved amino acids. In some embodiments, proteins homologous to BsPanD (SEQ ID NO: 37) comprise amino acids corresponding to at least 40%, at least 50%, at least 60%, at least 70%, at least 80%, at least 85%, at least 90%, at least 95%, or more than 95% of these highly conserved amino acids.

In some embodiments, the PanD comprises a eukaryotic PanD, such as TcPanD (SEQ ID NO: 38) or other eukaryotic PanD. The highly conserved amino acids in eukaryotic PanD are V88, P94, D102, L115, S126, V127, T129, H131, P132, F134, N136, Q137, L138, S140, D143, Y145, Q150, T153, D154, L156, N157, P158, S159, Y161, T162, E164, V165, P167, L171, M172, E173, E174, V176, L177, E179, M180, R181, 1183, G185, G191, G193, F195, P197, G198, G199, S200, A202, N203, G204, Y205, 1207, A210, R211, P216, K219, G222, L229, F232, T233, S234, E235, A237, H238, Y239, S240, K243, A245, F247, G249, G251, G264, P285, V288, T291, G293, T294, T295, V296, G298, A299, F300, D301, C310, K312, W316, H318, D320, A321, A322, W323, G324, G325, G326, A327, L328, S330, R334, L336, L337, G339, D344, S345, V346, T347, W348, N349, P350, H351, K352, L353, L354, A356, Q358, Q359, C360, S361, T362, L364, H367, L371, H375, A379, Y381, L382, F383, Q384, D386, K387, F388, Y389, D390, D394, G396, D397, H399, Q401, C402, G403, R404, A406, D407, V408, K410, F411, W412, M414, W415, A417, K418, G419, G422, H426, F431, R444, G446, P454, N458, F461, Y463, P465, R469, L481, A485, P486, K489, E490, M492, G496, M498, T501, Y502, Q503, N510, F511, F512, R513, V515, Q517, S519, L521, D525, M526, E532, E534, L536. In some embodiments, proteins homologous to TcPanD (SEQ ID NO: 38) comprise amino acids corresponding to at least 40%, at least 50%, at least 60%, at least 70%, at least 80%, at least 85%, at least 90%, at least 95%, or more than 95% of these highly conserved amino acids.

Some of the highly conserved amino acids in PanDs provided by the present disclosure are strictly conserved, and proteins homologous to a PanD of the present disclosure may comprise amino acid(s) corresponding to these strictly conserved amino acids.

Strictly conserved amino acids in bacterial PanDs such as the BsPanD (SEQ ID NO: 37) and the CgPanD (SEQ ID NO: 29) are K9, G24, S25, R54, and Y58. The ε-amine group on K9 is believed to form an ion pair with a-carboxyl group on aspartate, R54 is believed to form an ion pair with the γ-carboxyl group on aspartate, and Y58 is believed to donate a proton to an extended enolate reaction intermediate; thus, these three amino acids are important for aspartate binding and subsequent decarboxylation. Additionally, proteolytic cleavage between residues G24 and S25 produces an N-terminal pyruvoyl moiety also necessary for decarboxylase activity. Therefore, in some embodiments, bacterial enzymes suitable for use according to the present disclosure will comprise a substantial number, and sometimes all, of these strictly conserved amino acids corresponding to K9, G24, S25, R54, and Y58 in SEQ ID NOs: 37 and/or 29.

Strictly conserved amino acids in eukaryotic PanDs such as the TcPanD (SEQ ID NO: 38) are Q137, H238, K352, and R513. Q137 and R513 form a salt bridge with the γ-carboxyl group on aspartate, H238 is a base-stacking residue with the pyridine ring of the pyridoxal 5′-phosphate cofactor, and K352 forms a Schiff base linkage with the pyridoxal 5′-phosphate cofactor. Thus, these four amino acids are important for aspartate or cofactor binding and subsequent aspartate decarboxylation, and therefore, in some embodiments, eukaryotic enzymes suitable for use according to the present disclosure will comprise a substantial number, and sometimes all, of these strictly conserved amino acids corresponding to Q137, H238, K352, and R513 in SEQ ID NO: 38.

A PanD consensus sequence provides the sequence of amino acids in which each position identifies the amino acid (if a specific amino acid is identified) or a subset of amino acids (if a position is identified as variable) most likely to be found at a specific position in a PanD. The present disclosure provides two PanD consensus sequences—the bacterial PanD consensus sequence #4 (SEQ ID NO: 39) and the eukaryotic PanD consensus sequence #5 (SEQ ID NO: 40).

The bacterial PanD consensus sequence #4 (SEQ ID NO: 39) provides the sequence of amino acids in which each position identifies the amino acid (if a specific amino acid is identified) or a subset of amino acids (if a position is identified as variable) most likely to be found at a specific position in a bacterial PanD. Many amino acids in consensus sequence #4 (SEQ ID NO: 39) are highly conserved and bacterial PanDs suitable for use in accordance with the methods of the present disclosure will comprise a substantial number, and sometimes all, of these highly conserved amino acids at positions aligning with the location of the indicated amino acids in consensus sequence #4 (SEQ ID NO: 39). In various embodiments, proteins suitable for use in accordance with the methods of the present disclosure have PanD activity and comprise an amino acid sequence with at least 40%, at least 50%, at least 60%, at least 65%, or at least 70% sequence identity with consensus sequence #4 (SEQ ID NO: 39). For example, the BsPanD (SEQ ID NO: 37) is at least 40% identical to consensus sequence #4 (SEQ ID NO: 39), and is therefore encompassed by consensus sequence #4 (SEQ ID NO: 39).

In bacterial enzymes homologous to SEQ ID NO: 39, amino acids that are highly conserved are K9, H11, R12, A13, V15, T16, A18, L20, Y22, G24, S25, D29, E42, N51, G52, R54, T57, Y58, 160, G62, G65, G67, N72, G73, A74, A75, A76, G82, D83, V85, I86, Y90, E97, P103, and N112. In various embodiments, bacterial enzymes homologous to SEQ ID NO: 39 comprise at least 40%, at least 50%, at least 60%, at least 70%, at least 80%, at least 85%, at least 90%, at least 95%, or sometimes all of these highly conserved amino acids at positions corresponding to the highly conserved amino acids identified in SEQ ID NO: 39. For example, all of the highly conserved amino acids are found in SEQ ID NOs: 29 and 37.

Of the highly conserved amino acids, five of them are strictly conserved; K9, G24, S25, R54, and Y58 are important for PanD activity and are present in bacterial PanD consensus sequence SEQ ID NO: 39. The function of each strictly conserved amino acid, although the invention is not to be limited by any theory of mechanism of action, of each strictly conserved amino acid is as follows. The ε-amine group on K9 forms an ion pair with α-carboxyl group on aspartate, R54 is forms an ion pair with the γ-carboxyl group on aspartate, and Y58 donates a proton to an extended enolate reaction intermediate. Additional strictly conserved residues in SEQ ID NO: 39 are G24 and S25, and proteolytic cleavage between G24 and S25 results in production of an N-terminal pyruvoyl moiety required for decarboxylase activity. Bacterial enzymes homologous to consensus sequence SEQ ID NO: 39 comprise amino acids corresponding to all five of the strictly conserved amino acids identified in consensus sequence SEQ ID NO: 39. In some embodiments, each of these highly conserved amino acids are found in a desired PanD as provided, for example, in SEQ ID NO: 29, and SEQ ID NO: 37.

The eukaryotic PanD consensus sequence #5 (SEQ ID NO: 40) provides the sequence of amino acids in which each position identifies the amino acid (if a specific amino acid is identified) or a subset of amino acids (if a position is identified as variable) most likely to be found at a specific position in a eukaryotic PanD. Many amino acids in consensus sequence #5 (SEQ ID NO: 40) are highly conserved and bacterial PanDs suitable for use in accordance with the methods of the present disclosure will comprise a substantial number, and sometimes all, of these highly conserved amino acids at positions aligning with the location of the indicated amino acids in consensus sequence #5 (SEQ ID NO: 40). In various embodiments, proteins suitable for use in accordance with the methods of the present disclosure have PanD activity and comprise an amino acid sequence with at least 40%, at least 50%, at least 60%, at least 65%, or at least 70% sequence identity with consensus sequence #5 (SEQ ID NO: 40). For example, the TcPanD (SEQ ID NO: 38) is at least 40% identical to consensus sequence #5 (SEQ ID NO: 40), and is therefore encompassed by consensus sequence #5 (SEQ ID NO: 40).

In eukaryotic enzymes homologous to SEQ ID NO: 40, amino acids that are highly conserved are V130, P136, D144, L157, S168, V169, T171, H173, P174, F176, N178, Q179, L180, S182, D185, Y187, Q192, T195, D196, L198, N199, P200, S201, Y203, T204, E206, V207, P209, L213, M214, E215, E216, V218, L219, E221, M222, R223, 1225, G227, G234, G236, F238, P240, G241, G242, S243, A245, N246, G247, Y248, 1250, A253, R254, P259, K262, G265, L272, F275, T276, S277, E278, A280, H281, Y282, S283, K286, A288, F290, G292, G294, G307, P328, V331, T334, G336, T337, T338, V339, G341, A342, F343, D344, C353, K355, W359, H361, D363, A364, A365, W366, G367, G368, G369, A370, L371, S373, R377, L379, L380, G382, D387, S388, V389, T390, W391, N392, P393, H394, K395, L396, L397, A399, Q401, Q402, C403, S404, T405, L407, H410, L414, H418, A422, Y424, L425, F426, Q427, D429, K430, F431, Y432, D433, D437, G439, D440, H442, Q444, C445, G446, R447, A449, D450, V451, K453, F454, W455, M457, W458, A460, K461, G462, G465, H469, F474, R487, G489, P497, N501, F504, Y506, P508, R512, L525, A529, P530, K533, E534, M536, G540, M542, T545, Y546, Q547, N554, F555, F556, R557, V559, Q561, S563, L565, D569, M570, E576, E578, and L580. In various embodiments, eukaryotic enzymes homologous to SEQ ID NO: 40 comprise at least 40%, at least 50%, at least 60%, at least 70%, at least 80%, at least 85%, at least 90%, at least 95%, or sometimes all of these highly conserved amino acids at positions corresponding to the highly conserved amino acids identified in SEQ ID NO: 40. For example, all of these highly conserved amino acids are found in the TcPanD set forth in SEQ ID NO: 38.

Of the highly conserved amino acids, four of them are strictly conserved; Q179, H281, K395, and R557 are important for PanD activity and are present in eukaryotic PanD consensus sequence (SEQ ID NO: 40). The function of each strictly conserved amino acid, although the invention is not to be limited by any theory of mechanism of action, of each strictly conserved amino acid is as follows. Q179 and R557 form a salt bridge with the γ-carboxyl group on aspartate, H281 is a base-stacking residue with the pyridine ring of the pyridoxal 5′-phosphate cofactor, and K395 forms a Schiff base linkage with the pyridoxal 5′-phosphate cofactor. Thus, these four amino acids are important for aspartate or cofactor binding and subsequent aspartate decarboxylation. Eukaryotic enzymes homologous to consensus sequence SEQ ID NO: 40 comprise amino acids corresponding to all four strictly conserved amino acids identified in consensus sequence SEQ ID NO: 40. In some embodiments, each of these highly conserved amino acids are found in a desired PanD as provided, for example, in SEQ ID NO: 38.

2.4 Methods to Identify and/or Improve Enzymes in the Aspartic Acid Pathway and or the β-Alanine Pathway

The following exemplary methods have been developed for mutagenesis and diversification of genes for engineering specific or enhanced properties of targeted enzymes. Practitioners in the art will appreciate that the methods disclosed may be adapted as needed depending on the target enzyme properties desired. In some instances, the disclosed methods are suitable for use in engineering enzymes towards greater yield, titer and/or productivity of the aspartic acid pathway and/or the β-alanine pathway.

Methods described herein for protein mutagenesis, identification, expression, purification, and characterization are methods widely-practiced by practitioners skilled in the art, who will appreciate that a wide variety of commercial solutions are available for such endeavors. Practitioners will understand that identification of mutated proteins comprise activity screens and phenotypic selections.

### 2.4.1 Generating Protein Libraries Via Mutagenesis

Enzymes that are identified as good mutagenesis starting points enter the protein engineering cycle, which comprises protein mutagenesis, protein identification, protein expression, protein characterization, recombinant host cell characterization, and any combination thereof. Iterative rounds of protein engineering are typically performed to produce an enzyme variant with properties that are different from the template/original protein. Examples of enzyme characteristics that are improved and/or altered by protein engineering include, for example, substrate binding (Kₘ; a measure of enzyme binding affinity for a particular substrate) that includes non-natural substrate selectivity/specificity; enzymatic reaction rates (k_{cat}; the turnover rate of a particular enzyme-substrate complex into product and enzyme), to achieve desired pathway flux; temperature stability, for high temperature processing; pH stability, for processing in extreme pH ranges; substrate or product tolerance, to enable high product titers; removal of inhibition by products, substrates or intermediates; expression levels, to increase protein yields and overall pathway flux; oxygen stability, for operation of air sensitive enzymes under aerobic conditions; and anaerobic activity, for operation of an aerobic enzyme in the absence of oxygen. In some embodiments, the enzyme variant enables improved flux of the aspartic acid pathway and of the β-alanine pathway. In some embodiments, the enzyme variant enables increased aspartate yield, titer, and/or productivity and/or β-alanine yield, titer, and/or productivity. In some embodiments, the enzyme variant enables increased substrate specificity. In some embodiments, the enzyme variant displays improved kinetic properties, such as decreased Kₘ increased k_{cat}. In some embodiments, the enzyme variant has increased Kₘ and/or decreased k_{cat} for the substrate. In some embodiments, the enzyme variant has Kₘ≤3 mM. In some embodiments, the enzyme variant has k_{cat}≥10 turnovers per second. In some embodiments, the enzyme variant has decreased affinity for an allosteric inhibitor. In some embodiments, the enzyme variant is a product of one or more protein engineering cycles. In some embodiments, the enzyme variant comprises one or more point mutations.

In general, random and rational mutagenesis approaches are acceptable methods for generating DNA libraries of mutant proteins. Error-prone PCR is a random mutagenesis method widely used for generating diversity in protein engineering, and practitioners skilled in the art will recognize that error-prone PCR is not only fast and easy, but it is also a method that has successfully produced mutated enzymes with altered activity from a wild type DNA template. (Wilson, D. S. & Keefe, A. D. Random mutagenesis by PCR. Curr. Protoc. Mol. Biol. Chapter 8, Unit 8.3 (2001.) To help increase the odds of identifying an enzyme with desired improved activity, rational mutagenesis of a small number of active site mutations is also useful. Practitioners in the art will appreciate that structural modeling allows one to identify amino acids in the active site believed to be important for substrate recognition. Other mutagenesis approaches that could be used include DNA shuffling and combinatorial mutagenesis. In some embodiments, the mutagenesis step is carried out more than once, resulting in iterative rounds of engineering.

### 2.4.2 Enzyme Characterization

Protein variants that result from mutagenesis are integrated into the genome of recombinant host cells and resulting strain variants are analyzed for aspartic acid pathway and/or β-alanine pathway activity. In some embodiments, iterative rounds of protein engineering are performed to produce enzyme variants with optimized properties, wherein the iterative rounds of protein engineering comprise rational mutagenesis and random mutagenesis. In these embodiments, select variants from preceding rounds of protein engineering are identified for further protein engineering. Non-limiting examples of such properties comprise improved enzyme kinetics for specificity and/or turnover, improved pathway flux, increased metabolite yield, decreased inhibitor affinity, and decreased byproduct yield. In some embodiments, culture medium or fermentation broth is analyzed for the presence of metabolites such as aspartic acid, β-alanine, and/or byproducts, wherein the method of analysis is HPLC (high-performance liquid chromatography).

### 2.5 Ancillary Proteins

In addition to the aspartic acid and/or β-alanine pathway enzymes, ancillary proteins are other proteins that are overexpressed in recombinant host cells of the present disclosure whose overexpression results in an increase in aspartic acid and/or β-alanine as compared to control, or host cells that do not overexpress said proteins. Ancillary proteins function outside the aspartic acid and/or β-alanine pathway, wherein each ancillary protein plays a role that indirectly boosts the recombinant host cell's ability to produce aspartic acid and/or β-alanine. Ancillary proteins comprise any protein (excluding aspartic acid pathway enzymes and β-alanine pathway enzymes) of any structure or function that can increase aspartic acid and/or β-alanine yields, titers, or productivities when overexpressed. Non-limiting examples of classes of proteins include transcription factors, transporters, scaffold proteins, proteins that decrease byproduct accumulation, and proteins that regenerate or synthesize redox cofactors.

Provided herein in certain embodiments are recombinant host cells comprising one or more heterologous nucleic acids encoding one or more ancillary proteins wherein said recombinant host cell is capable of producing higher aspartic acid and/or β-alanine yields, titers, or productivities as compared to control cells, or host cells that do not comprise said heterologous nucleic acid(s). In some embodiments, that host recombinant cell naturally produces aspartic acid and/or β-alanine, and in these cases, the aspartic acid and/or β-alanine yields, titers, and/or productivities are increased. In other embodiments, the recombinant host cell comprises one or more heterologous nucleic acids encoding one or more aspartic acid and/or β-alanine pathway enzymes.

In certain embodiments of the present disclosure, the recombinant host cells comprise one or more heterologous nucleic acids encoding one or more aspartic acid and/or β-alanine pathway enzymes and one or more heterologous nucleic acids encoding one or more ancillary proteins. In certain of these embodiments, the recombinant host cells may be engineered to express more of these ancillary proteins. In these particular embodiments, the ancillary proteins are expressed at a higher level (i.e., produced at a higher amount as compared to cells that do not express said ancillary proteins) and may be operatively linked to one or more exogenous promoters or other regulatory elements.

In certain embodiments, recombinant host cells comprise both endogenous and heterologous nucleic acids encoding one or more aspartic acid and/or β-alanine pathway enzymes and one or more ancillary proteins. In certain embodiments, the recombinant host cells comprise one or more heterologous nucleic acids encoding one or more aspartic acid and/or β-alanine pathway enzymes and/or one or more ancillary proteins, and one or more endogenous nucleic acids encoding one or more aspartic acid and/or β-alanine pathway enzymes and/or one or more ancillary proteins.

In certain embodiments, endogenous nucleic acids of ancillary proteins are modified in situ (i.e., on chromosome in the host cell genome) to alter levels of expression, activity, or specificity. In some embodiments, heterologous nucleic acids are inserted into endogenous nucleic acids of ancillary proteins.

### 2.5.1 Ancillary Proteins for Redox Cofactor Recycling and Biogenesis

One type of ancillary protein are proteins that recycle the redox cofactors that are produced during aspartic acid and/or β-alanine pathway activity. Redox balance is fundamental to sustained metabolism and cellular growth in living organisms. Intracellular redox potential is determined by redox cofactors that facilitate the transfer of electrons from one molecule to another within a cell. Redox cofactors in yeast include the nicotinamide adenine dinucleotides, NAD and NADP, the flavin nucleotides, FAD and FMN, and iron sulfur clusters (Fe—S clusters).

Redox constraints play an important role in end-product formation. Additional reducing power must be provided to produce compounds whose degree of reduction is higher than that of the substrate. Conversely, producing compounds with a degree of reduction lower than that of the substrate will force the synthesis of byproducts with higher degrees of reduction to compensate for excess reducing power generated from substrate oxidation. Thus, redox neutrality must be maintained to ensure high end-product yields. For example, the aspartic acid and/or β-alanine pathway is redox balanced from glucose and there is no net formation of NAD(P)⁺ or NAD(P)H for each mol of glucose stoichiometrically converted to aspartic acid and/or β-alanine in the cytosol.

The NAD and NADP cofactors are involved in electron transfer and contribute to approximately 12% of all biochemical reactions in a cell (Osterman A., EcoSal Plus, 2009). NAD is assembled from aspartate, dihydroxyacetone phosphate (DHAP; glycerone), phosphoribosyl pyrophosphate (PRPP) and ATP. The NADP is assembled in the same manner and further phosphorylated. In some embodiments, recombinant host cells comprise heterologous and/or endogenous nucleic acids encoding one or more ancillary proteins that facilitate NAD and NADP cofactor assembly. In some embodiments, the ancillary proteins comprise one, more or all proteins suitable for use in accordance with methods of the present disclosure having NAD and/or NADP assembly capability, NAD and/or NADP transfer capability, NAD and/or NADP chaperone capability, or any combination thereof.

Similarly, Fe—S clusters facilitate various enzyme activities that require electron transfer. Because both iron and sulfur atoms are highly reactive and toxic to cells, Fe—S cluster assembly requires carefully coordinated synthetic pathways in living cells. The three known pathways are the Isc (iron sulfur cluster) system, the Suf (sulfur formation) system, and the Nif (nitrogen fixation) system. Each of these systems has a unique physiological role, yet several functional components are shared between them. First, a cysteine desulfurase enzyme liberates sulfur atoms from free cysteine. Then, a scaffold protein receives the liberated sulfur for Fe—S cluster assembly. Finally, the Fe—S cluster is transferred to a target apoprotein. In some embodiments of the present disclosure, recombinant host cells comprise heterologous and/or endogenous nucleic acids encoding one or more ancillary proteins that facilitate Fe—S cluster assembly. In some embodiments, the ancillary proteins comprise one, more or all proteins of the Isc system, the Suf system, the Nif system, or any combination thereof. In some embodiments, recombinant host cells comprise one or more heterologous nucleic acids encoding one or more proteins suitable for use in accordance with methods of the present disclosure having cysteine desulfurase activity, Fe—S cluster assembly capability, Fe—S cluster transfer capability, iron chaperone capability, or any combination thereof.

### 2.5.1.1 Glutamate Dehydrogenase

In embodiments wherein recombinant host cells comprise heterologous nucleic acids encoding an AspB to produce aspartate and/or β-alanine, the recombinant host cells may further comprise heterologous nucleic acids encoding a glutamate dehydrogenase (GDH; EC #1.4.1.2 or 1.4.1.3). AspB converts one molecule of oxaloacetate and one molecule of glutamate to one molecule of aspartate and one molecule of oxoglutarate (FIG. 1 and Table 1). In the aspartic acid and β-alanine pathways of the present disclosure, the oxoglutarate generated by AspB (section 2.2.2.2, FIG. 1 and Table 1) needs to be converted back to glutamate for future AspB reactions so that the aspartic acid/β-alanine pathway does not become disrupted. GDH enables this oxoglutarate-glutamate recycling with concomitant oxidation of NAD(P)H to NAD(P)⁺.

GDH comes in two types: NAD⁺-dependent GDH (EC #1.4.1.2) and NAD(P)⁺-dependent GDH (EC #1.4.1.3). The NAD⁺-dependent GDH (EC #1.4.1.2) converts one molecule of oxoglutarate, one molecule of ammonia, one proton, and one molecule of NADH to one molecule of glutamate, one molecule of water, and one molecule of NAD⁺. The NAD(P)⁺-dependent GDH (EC #1.4.1.3) utilizes converts one molecule of oxoglutarate, one molecule of ammonia, one molecule of NADPH or NADH, and one proton to one molecule of glutamate, one molecule of water, and one molecule of NADP⁺ or NAD⁺. In various embodiments, proteins suitable for use in accordance with methods of the present disclosure have either EC #1.4.1.2 or EC #1.4.1.3 GDH activity. In many embodiments, the recombinant host cell is a C. glutamicum strain.

As disclosed above in section 2.2.2.1 on AspDH, NADH is generally produced during a recombinant host cell's glycolytic processes in converting glucose to pyruvate. In C. glutamicum, P. kudriavzevii, S. cerevisiae, P. ananatis, and E. coli, for example, the glyceraldehyde 3-phosphate dehydrogenase (GAPDH) in glycolysis reduces NAD⁺ to NADH; therefore, in embodiments wherein GAPDH produces NADH, the GDH is NADH-utilizing (EC #1.4.1.2 or 1.4.1.3) to ensure aspartate transaminase turnover is not impeded as GDH is able to utilize readily available NADH. Similarly, in other embodiments wherein the GAPDH enzyme produces NADPH, the GDH is NADPH-utilizing (EC #1.4.1.3).

In many embodiments, the GDH is derived from a prokaryotic source. In many of these embodiments, the GDH is derived from a host cell belonging to a genus selected from the group comprising Bacillus, Clostridium, Corynebacterium, Escherichia, Helicobacter, Methanocaldococcus, Mycobacterium, Peptoniphilus, Pyrococcus, Rhodobacter, Salmonella, Thermococcus and Thermus. In some embodiments, the GDH is selected from the group consisting the Clostridium symbiosum UniProt ID: U2D2C5 (abbv. CsGDH; SEQ ID NO: 52), Corynebacterium glutamicum UniProt ID: P31026 (abbv. CgGDH; SEQ ID NO: 53), and the Peptoniphilus asaccharolyticus UniProt ID: P28997 (abbv. PaGDH; SEQ ID NO: 54).

In many embodiments, recombinant host cells comprise one or more heterologous nucleic acids encoding a GDH wherein said recombinant host cells are capable of producing aspartic acid and/or β-alanine. In various embodiments, proteins suitable for use in accordance with methods of the present disclosure have GDH activity and comprise an amino acid sequence with at least 40%, at least 50%, at least 60%, at least 70%, at least 80%, at least 90%, or at least 95% sequence identity with SEQ ID NO: 52, SEQ ID NO: 53, or SEQ ID NO: 54. In many embodiments, the recombinant host cell is a C. glutamicum strain.

In some embodiments, recombinant host cells comprise one or more heterologous nucleic acids encoding an GDH wherein the GDH was mutagenized towards an altered enzyme characteristic such as altered substrate affinity, cofactor affinity, altered reaction rate, and/or altered inhibitor affinity. In these embodiments, the GDH variant is a product of one or more protein engineering cycles. In these embodiments, the GDH variant comprises one or more point mutations. In these embodiments, proteins suitable for use in accordance with methods of the present disclosure have GDH activity and comprise an amino acid sequence with at least 40%, at least 50%, at least 60%, at least 70%, at least 80%, at least 90%, or at least 95% sequence identity with SEQ ID NO: 52, SEQ ID NO: 53, or SEQ ID NO: 54. In some of these embodiments, the GDH variant has increased affinity for NADH. In many embodiments, the recombinant host cell is a C. glutamicum strain.

### 2.5.1.2 NADP⁺-Utilizing Glyceraldehyde 3-Phosphate Dehydrogenase

Buildup of oxidized cofactor, i.e., NAD⁺ or NADP⁺, is inherent to the aspartic acid and β-alanine pathways of the present disclosure at the step catalyzed by aspartate dehydrogenase (AspDH) (FIG. 1 and Table 1; Section 2.2.2.1). Reduction of NAD(P)⁺ back to NAD(P)H can help ensure pathway flux is not impeded by NAD(P)H depletion.

In embodiments wherein recombinant host cells comprise heterologous nucleic acids encoding an AspDH that utilizes NADPH, the recombinant host cells further comprise heterologous nucleic acids encoding a NADP⁺-utilizing glyceraldehyde 3-phosphate dehydrogenase (GAPDH). In said recombinant host cells, the native GAPDH is NAD⁺-utilizing; native GAPDH converts one molecule of glyceraldehyde 3-phosphate, one molecule of phosphate and one molecule of NAD⁺ to one molecule of 3-phosphoglycerol phosphate, one molecule of NADH and one molecule of H⁺. In said recombinant host cells, the heterologous NADP⁺-utilizing GAPDH would carry out the same reaction as the native GAPDH, except that it would utilize NADP⁺ instead of NAD⁺. In many embodiments, the recombinant host cell is a C. glutamicum strain. In many embodiments, the NADP⁺-utilizing GAPDH is derived from a bacterial source. In many embodiments, the NADP⁺-utilizing GAPDH is derived from the group comprising Bacillus sp., Clostridium pasteurianum, Streptococcus pyogenes, Kluyveromyces lactis, Methanococcus maripaludis, Streptomyces microflavus, Vibrio sp., Corynebacterium casei, Psychrobacter aquaticus, Micrococcus lylae, Escherichia coli, Streptococcus mutans or Clostridium acetobutylium. Non-limiting examples of bacterial NADP⁺-utilizing GAPDH include Kluyveromyces lactis UniProt ID: Q8J0C9, Methanococcus maripaludis UniProt ID: Q6M0E6 (abbv. MmGapC), Streptococcus pyogenes UniProt ID: A0A0H2UV68, Clostridium pasteurianum UniProt ID: A0A1D9N2A5, Bacillus sp. dmp5 UniProt ID: A0A371VHU2, Clostridium acetobutylicum UniProt ID: Q97D25 (abbv. CaGapC), Streptomyces microflavus UniProt ID: A0A285D866, Vibrio sp. JB196 UniProt ID: A0A1R4J356, Corynebacterium casei LMG UniProt ID: W5XUZ7, Psychrobacter aquaticus CMS 56 UniProt ID: U4T4I2, Micrococcus lylae UniProt ID: A0A1R4JAC2, and Streptococcus mutans UniProt ID: Q59931.

In some embodiments, the bacterial NADP⁺-utilizing GAPDH is the Kluyveromyces lactis UniProt ID: Q8J0C9. In some embodiments, the bacterial NADP⁺-utilizing GAPDH is the Methanococcus maripaludis UniProt ID: Q6M0E6 (abbv. MmGapC). In some embodiments, the bacterial NADP⁺-utilizing GAPDH is the Streptococcus pyogenes UniProt ID: A0A0H2UV68. In some embodiments, the bacterial NADP⁺-utilizing GAPDH is the Clostridium pasteurianum UniProt ID: A0A1D9N2A5. In some embodiments, the bacterial NADP⁺-utilizing GAPDH is the Bacillus sp. dmp5 UniProt ID: A0A371VHU2. In some embodiments, the bacterial NADP⁺-utilizing GAPDH is the Clostridium acetobutylicum UniProt ID: Q97D25 (abbv. CaGapC). In some embodiments, the bacterial NADP⁺-utilizing GAPDH is the Streptomyces microflavus UniProt ID: A0A285D866. In some embodiments, the bacterial NADP⁺-utilizing GAPDH is the Vibrio sp. JB196 UniProt ID: A0A1R4J356. In some embodiments, the bacterial NADP⁺-utilizing GAPDH is the Corynebacterium casei LMG UniProt ID: W5XUZ7. In some embodiments, the bacterial NADP⁺-utilizing GAPDH is the Psychrobacter aquaticus CMS 56 UniProt ID: U4T4I2. In some embodiments, the bacterial NADP⁺-utilizing GAPDH is the Micrococcus lylae UniProt ID: A0A1R4JAC2. In some embodiments, the bacterial NADP⁺-utilizing GAPDH is the Streptococcus mutans UniProt ID: Q59931.

In many embodiments, recombinant host cells comprise one or more heterologous nucleic acids encoding a NADP⁺-utilizing GAPDH wherein said recombinant host cells are capable of producing aspartic acid and/or β-alanine. In various embodiments, proteins suitable for use in accordance with methods of the present disclosure have NADP⁺-utilizing GAPDH activity and comprise an amino acid sequence with at least 40%, at least 50%, at least 60%, at least 70%, at least 80%, at least 90%, or at least 95% sequence identity with UniProt ID: Q8J0C9, UniProt ID: Q6M0E6, UniProt ID: A0A0H2UV68, UniProt ID: A0A1D9N2A5, UniProt ID: A0A371VHU2, UniProt ID: Q97D25, UniProt ID: A0A285D866, UniProt ID: A0A1R4J356, UniProt ID: W5XUZ7, UniProt ID: U4T4I2, UniProt ID: A0A1R4JAC2, or UniProt ID: Q59931. In many embodiments, the recombinant host cell is a C. glutamicum strain. Examples 11 and 12 describe recombinant host cells of the present disclosure comprising NADP⁺-utilizing GAPDH that demonstrated improved aspartic acid production.

In some embodiments, in addition to comprising one or more heterologous nucleic acids encoding a NADP⁺-utilizing GAPDH, the recombinant host cells further comprise disruption of a native NAD⁺-dependent GADPH. In these embodiments, the recombinant host cells are capable of producing more aspartic acid and/or β-alanine than cells without disruption of a native NAD⁺-dependent GADPH. In embodiments where the recombinant host cell is a C. glutamicum strain, a native NAD⁺-dependent GAPDH that is disrupted is UniProt ID: A0A0U4IQV8 (abbv. CgGapX). In embodiments where the recombinant host cell is an E. coli strain, a native NAD⁺-dependent GAPDH that is disrupted is UniProt ID: P0A9B2 (abbv. EcGapA).

### 2.5.2 Ancillary Proteins for Aspartic Acid Transport

Another class of ancillary proteins useful for increasing aspartic acid yields, titers, and/or productivities is an amino acid transporter capable of transporting aspartic acid. In some embodiments, recombinant host cells comprise one or more heterologous and/or endogenous nucleic acids encoding one or more amino acid transporters. In many embodiments, the amino acid transporter is derived from a prokaryotic source. In many embodiments, the amino acid transporter is derived from a eukaryotic source. In some embodiments, the amino acid transporter is selected from the group comprising Saccharomyces cerevisiae PDR12 (abbv. ScPDR12; UniProt ID: Q02785; SEQ ID NO: 30), Saccharomyces cerevisiae WAR1 (abbv. ScWAR1; UniProt ID: Q03631; SEQ ID NO: 31), Schizosaccharomyces pombe MAE1 (abbv. SpMAE1; UniProt ID; P50537; SEQ ID NO: 32), Kluyveromyces marxianus PDR12 (abbv. KmPDR12; UniProt ID: W0T9C6; SEQ ID NO: 7), Corynebacterium glutamicum GLUD (abbv. CgGLUD; UniProt ID: P48245; SEQ ID NO: 41), Corynebacterium glutamicum GLUA (abbv. CgGLUA; UniProt ID: P48243; SEQ ID NO: 42), and Corynebacterium glutamicum GLUC (abbv. CgGLUC; UniProt ID: P48244; SEQ ID NO: 43).

In some embodiments, recombinant host cells comprise one or more heterologous nucleic acids encoding one or more proteins with aspartic acid transporter activity, i.e., capable of transporting aspartate or aspartic acid across a cell membrane. In some embodiments, recombinant host cells comprise one or more heterologous nucleic acids encoding one or more proteins that comprise an amino acid sequence with at least 40%, at least 50%, at least 60%, at least 70%, at least 80%, at least 90%, or at least 95% sequence identity with ScPDR12 (SEQ ID NO: 30), ScWAR1 (SEQ ID NO: 31), SpMAE1 (SEQ ID NO: 32), or KmPDR12 (SEQ ID NO: 7), CgGLUD (SEQ ID NO: 41), CgGLUA (SEQ ID NO: 42), or CgGLUC (SEQ ID NO: 43).

### 2.5.3 Ancillary Proteins for Carbon Fixation

In the aspartic acid and β-alanine pathways of the present disclosure, one molecule of CO₂ is fixed with the conversion of each molecule of glucose to aspartate or β-alanine (FIG. 1). The reaction steps involved are catalyzed by the oxaloacetate-forming enzymes: pyruvate carboxylase (PYC), phosphoenolpyruvate carboxykinase (PCK), and/or phosphoenolpyruvate carboxylase (PPC) (Table 1). Carbon dioxide diffuses across cell membranes and is converted to HCO₃⁻, which serves as the co-substrate for an oxaloacetate-forming enzyme, namely PYC, PCK, and/or PPC. An abundant pool of HCO₃⁻ helps the oxaloacetate-forming enzyme reactions move forward and prevents these steps in the aspartic acid and β-alanine pathways from becoming a bottleneck of the pathways. Carbonic anhydrase (EC #4.2.1.1) is a carbon fixation enzyme that accelerates the rate CO₂ conversion to HCO₃⁻ and as such it is an important ancillary protein for ensuring HCO₃⁻ availability does not limit the rate of oxaloacetate-forming enzyme activity. Thus, in some embodiments, the ancillary proteins useful for increasing aspartate or β-alanine product yields, titers, and/or productivities are carbon fixation enzymes. In some embodiments wherein recombinant host cells comprise heterologous nucleic acids expressing one or more carbonic anhydrases, the recombinant host cells have higher aspartic acid or β-alanine yields, titers, and/or productivities. In some embodiments, the carbonic anhydrase is derived from a prokaryotic source. In some embodiments, the carbonic anhydrase is derived from a eukaryotic source.

In some embodiments, the carbonic anhydrase is selected from the group comprising Homo sapiens carbonic anhydrase (abbv. HsCAH; UniProt ID: P00918; SEQ ID NO: 44), Flaveria bidentis carbonic anhydrase (abbv. FbCAH; UniProt ID: P46510; SEQ ID NO: 45), Saccharomyces cerevisiae carbonic anhydrase (abbv. ScCAH; UniProt ID: P53615; SEQ ID NO: 46), Candida albicans carbonic anhydrase (abbv. CaCAH; UniProt ID: Q5AJ71; SEQ ID NO: 47), Porphyromonas gingivalis carbonic anhydrase (abbv. PgCAH; UniProt ID: Q7MV79; SEQ ID NO: 48), Mycobacterium tuberculosis carbonic anhydrase (abbv. MtCAH; UniProt ID: P9WPJ9; SEQ ID NO: 49), Escherichia coli carbonic anhydrase 1 (abbv. EcCAH1; UniProt ID: P0ABE9), and Escherichia coli carbonic anhydrase 2 (abbv. EcCAH2; UniProt ID: P615517).

In some embodiments, recombinant host cells comprise one or more heterologous nucleic acids encoding one or more proteins suitable for use in accordance with methods of the present disclosure have carbonic anhydrase activity. In some embodiments, recombinant host cells comprise one or more heterologous nucleic acids encoding one or more proteins that comprise an amino acid sequence with at least 40%, at least 50%, at least 60%, at least 70%, at least 80%, at least 90%, or at least 95% sequence identity with HsCAH (SEQ ID NO: 44), FbCAH (SEQ ID NO: 45), ScCAH (SEQ ID NO: 46), CaCAH (SEQ ID NO: 47), PgCAH (SEQ ID NO: 48), MtCAH (SEQ ID NO: 49), EcCAH1 (SEQ ID NO: 50), or EcCAH2 (SEQ ID NO: 51).

### 2.6 Decreasing or Eliminating Expression of Byproduct Pathway Enzymes

In an additional aspect of the invention, nucleic acids encoding byproduct pathway enzymes are disrupted in recombinant host cells of the present disclosure to increase aspartic acid and/or β-alanine yields, productivities, and/or titers; and/or to decrease byproduct titers and/or yields as compared to control cells, or host cells that express native/undisrupted levels of said byproduct pathway enzymes. Byproduct pathway enzymes comprise any native protein (excluding aspartic acid and/or β-alanine pathway enzymes) of any structure or function that can increase aspartic acid and/or β-alanine product yields, titers, and/or productivities when disrupted because they utilize intermediates or products of the aspartic acid and/or β-alanine pathway. In addition, byproduct pathway enzymes also comprise any native protein (excluding aspartic acid and/or β-alanine pathway enzymes) of any structure or function that can decrease undesired byproduct yields, titers, and/or productivities when disrupted because they utilize intermediates or products of the aspartic acid and/or β-alanine pathway.

Byproducts that accumulate during aspartic acid and/or β-alanine production can lead to: (1) lower aspartic acid and/or β-alanine titers, productivities, and/or yields; and/or (2) accumulation of byproducts in the fermentation broth that increase the difficulty of downstream purification processes. In some embodiments, recombinant host cells may comprise genetic disruptions that encompass alterations, deletions, knockouts, substitutions, promoter modifications, premature stop codons, or knock-downs that decrease byproduct accumulation. In some embodiments, recombinant host cells comprising a disruption of one or more genes encoding a byproduct pathway enzyme will have altered performance characteristics as compared to cells without said genetic disruption(s), such as decreased or eliminated byproduct pathway enzyme expression, decreased or eliminated byproduct accumulation, improved aspartic acid and/or β-alanine activity, altered metabolite flux through the aspartic acid and/or β-alanine pathway, higher aspartic acid and/or β-alanine titers, productivities, yields, and/or altered cellular fitness.

One important reason to decrease byproduct formation is to increase aspartic acid and/or β-alanine pathway activity, resulting in an increased amount of aspartic acid and/or β-alanine produced. In many embodiments, recombinant host cells of the present disclosure comprising one or more genetic disruptions of one or more genes encoding a byproduct pathway enzyme produce an increased aspartic acid and/or β-alanine titer as compared to host cells that do not comprise said genetic disruption(s). In some of these embodiments, the aspartic acid and/or β-alanine titer in the fermentation broth is increased by 0.5 g/l, 1 g/l, 2.5 g/l, 5 g/l, 7.5 g/l, 10 g/l, or more than 10 g/l.

In addition to increasing aspartic acid and/or β-alanine titers, decreasing byproduct formation can also help increase aspartic acid and/or β-alanine yields. Because yield is independent of the volume of the fermentation broth, which can change during the course of a fermentation, it is often advantageous to measure aspartic acid and/or β-alanine yields. In many embodiments, recombinant host cells of the present disclosure comprising one or more genetic disruptions of one or more genes encoding byproduct pathway enzymes produce an increased aspartic acid and/or β-alanine yield as compared to host cells that do not comprise said genetic disruption. In some of these embodiments, the aspartic acid and/or β-alanine yield is increased by 0.5%, 1%, 2.5%, 5%, 7.5%, 10%, or more than 10% (g-aspartic acid/g-substrate, and/or β-alanine/g-substrate). The substrate in this yield calculation is the fermentation substrate, which is typically glucose, but may also be other, non-glucose substrates (e.g., sucrose, glycerol, or pyruvate).

Increasing aspartic acid and/or β-alanine is important for decreasing manufacturing costs, but it is also useful to disrupt genes encoding byproduct pathway enzymes in order to decrease byproduct formation. Byproducts are typically unwanted chemicals, are disposed of as waste, and their disposal can involve elaborate processing steps and containment requirements. Therefore, decreasing byproduct formation is generally also important for lowering production costs. In many embodiments, recombinant host cells of the present invention comprising one or more genetic disruptions of one or more genes encoding a byproduct pathway enzyme produces a lower byproduct titer as compared to host cells that do not comprise said genetic disruption. In some of these embodiments, a recombinant host cell of the disclosure comprising genetic disruption of one or more byproduct pathway enzymes produces a byproduct titer that is 0.5 g/l, 1 g/l, 2.5 g/l, 5 g/l, 7.5 g/l, 10 g/l, or greater than 10 g/l less than host cells that do not comprise said genetic disruption.

In many embodiments, recombinant host cells of the present disclosure comprising one or more genetic disruptions of one or more genes encoding a byproduct pathway enzyme produces a lower byproduct yield as compared to host cells that do not comprise said genetic disruption(s). In some of these embodiments, recombinant host cells comprise genetic disruption of one or more genes encoding byproduct pathway enzymes produce a byproduct yield that is 0.5%, 1%, 2.5%, 5%, 7.5%, 10%, or greater than 10% (g-byproduct/g-substrate) less than host cells that do not comprise said genetic disruption. As with the aspartic acid and/or β-alanine yield calculation, the substrate used in the byproduct yield calculation is the carbon source provided to the fermentation; this is typically glucose, sucrose, or glycerol, but may be any carbon substrate.

Non-limiting examples of byproducts that arise due to consumption of an aspartic acid and/or β-alanine pathway or a downstream pathway substrate, intermediate or product include lactate, L-alanine, malate, and succinate. In the event of a redox imbalance, an undesirable excess of reduced or oxidized cofactors may also accumulate; thus, under many circumstances the redox cofactors NADH, NAD⁺, NADPH and NADP⁺ can also be considered byproducts.

A non-limiting list of enzyme-catalyzed reactions that utilize the aspartic acid and/or β-alanine pathway substrates or intermediates are found in Table 2. Decreasing or eliminating expression of one, some or all of the genes encoding the enzymes in Table 2 can increase aspartic acid and/or β-alanine production and/or decrease byproduct production. In many cases, the product of the enzyme-catalyzed reactions provided in Table 2 can accumulate in the fermentation broth; in such cases, this indicates that expression of the native gene encoding the listed enzyme should be reduced or eliminated. For example, the occurrence of lactate in the fermentation broth indicates that expression of a native gene encoding lactate dehydrogenase should be decreased or eliminated. In some cases, the product of the specific reaction listed in Table 2 is further converted, either spontaneously or through the action of other enzymes, into a byproduct that accumulates in the fermentation broth. In cases where byproduct accumulation is due to the activity of multiple enzymes, one or more of the genes encoding the one or more byproduct pathway enzymes can be deleted or disrupted to reduce byproduct formation.

TABLE 2
ENZYME-CATALYZED REACTIONS THAT CONSUME A SUBSTRATE,
INTERMEDIATE OR PRODUCT OF GLYCOLYSIS, A ASPARTIC
ACID PATHWAY, AND/OR A β-ALANINE PATHWAY

| Substrate | EC # | Enzyme name | Reaction formula |
|---|---|---|---|
| Pyruvate | 1.1.1.27 | Lactate | Pyruvate + NAD⁺ → Lactate + NADH |
| | | dehydrogenase | |
| Fumarate | 1.3.5.1 | Succinate | Fumarate + Quinol → Succinate + |
| | | dehydrogenase | Quinone |
| Pyruvate | 2.6.1.2 | Alanine | Pyruvate + L-Glutamate → L-Alanine + |
| | | transaminase | 2-Oxoglutarate |
| Pyruvate + | 1.1.1.37 | Malate | Pyruvate + Oxaloacatate + NAD(P)H → |
| Oxaloacetate | | dehydrogenase | Malate + NAD(P)⁺ |
| Pyruvate | 1.4.1.1 | Alanine | Pyruvate + NH3 + NADH + H+ → L- |
| | | dehydrogenase | alanine + H2O + NAD+ |

### 2.6.1 Decreasing or Eliminating Expression of Lactate Dehydrogenase

It is beneficial to decrease or eliminate expression of lactate dehydrogenase to decrease lactate byproduct titer, thereby preventing carbon flux from leaving the aspartic acid/β-alanine pathways.

Lactate dehydrogenase (EC #1.1.1.27) catalyzes the aspartic acid/β-alanine pathway intermediate pyruvate to lactate with concomitant oxidation of NADH to NAD⁺ (Table 2). Thus, the expression of endogenous lactate dehydrogenase can decrease anaerobic (or oxygen limited) production of aspartic acid and/or β-alanine. Any enzyme is suitable for use in accordance with the invention so long as the enzyme is capable of catalyzing said lactate dehydrogenase reaction. Genetic disruption of native nucleic acids that encode lactate dehydrogenase is useful for increasing aspartic acid and/or β-alanine titers, yields, and/or productivities. In some embodiments, the recombinant host cell is a Corynebacterium glutamicum strain.

In some embodiments, recombinant host cells comprise heterologous nucleic acids encoding an aspartic acid pathway and/or a β-alanine pathway, and further comprise genetic disruptions to decrease or eliminate expression of lactate dehydrogenase. In some embodiments, the lactate dehydrogenase is the C. glutamicum lactate dehydrogenase UniProt ID: Q9HYA4 (abbv. CgLDHA; SEQ ID NO: 1). In some embodiments, recombinant host cells comprise genetic disruptions of a homologous lactate dehydrogenase gene with least 40%, at least 50%, at least 60%, at least 70%, at least 80%, at least 90%, at least 95%, or more than 95% homology when compared to SEQ ID NO: 1.

In some embodiments, recombinant host cells comprising heterologous nucleic acids encoding aspartic acid and/or β-alanine pathway enzymes, and genetic disruption of a native lactate dehydrogenase homolog will further comprise a lactate byproduct titer of 10 g/l or less, preferably 1 g/l or less, and most preferably 0.5 g/l or less. In certain embodiments, lactate byproduct yield (i.e., percentage of g of byproduct/g of substrate at the end of fermentation) is 10% or less, 5% or less, 2.5% or less, and preferably, 1% or less.

In some embodiments, recombinant host cells comprising heterologous nucleic acids encoding aspartic acid pathway enzymes and genetic disruption of a native lactate dehydrogenase homolog will further comprise higher aspartate yield, titer, and/or productivity than cells lacking genetic disruption of a lactate dehydrogenase homolog. In some embodiments, recombinant host cells comprising heterologous nucleic acids encoding β-alanine pathway enzymes and genetic disruption of a native lactate dehydrogenase homolog will further comprise higher β-alanine yield, titer, and/or productivity than cells lacking genetic disruption of a lactate dehydrogenase homolog.

The construction of recombinant host cells comprising a genetically disrupted lactate dehydrogenase is described below in Examples 1 and 3. The titers for lactate, succinate and aspartic acid of these recombinant host cells are described below in Examples 5 and 8.

### 2.6.2 Decreasing or Eliminating Expression of Succinate Dehydrogenase

It is beneficial to decrease or eliminate expression of succinate dehydrogenase to decrease succinate byproduct titer, thereby preventing carbon flux from leaving the aspartic acid/β-alanine pathways.

Succinate dehydrogenase (EC #1.3.5.1) functions in the tricarboxylic acid (abbv. TCA) cycle (which is synonymous with citric acid cycle) where it catalyzes the reversible conversion of one molecule of fumarate and one molecule of quinol to one molecule of succinate and one molecule of quinone (Table 2). When the TCA cycle is active, oxaloacetate is directed from the aspartic acid and β-alanine pathways (Table 1 and FIG. 1) to function as a TCA cycle intermediate, enabling the cell to oxidize acetyl-CoA for the production of ATP and NADH. Under anaerobic conditions during aspartic acid/β-alanine production phase, the TCA cycle flows in the reductive direction, resulting in a buildup of succinate. Genetic disruption of native nucleic acids that encode the succinate dehydrogenase decreases succinate byproduct, disables the TCA cycle, and is useful for ensuring oxaloacetate is available for the aspartic acid and β-alanine pathways, thereby increasing aspartic acid/β-alanine yields, titers and/or productivities. In some embodiments, any enzyme is suitable so long as the enzyme is capable of catalyzing said succinate dehydrogenase reaction. In some embodiments, any enzyme is suitable for use in accordance with the invention so long as the enzyme functions in the TCA cycle. In some embodiments, the recombinant host cell is a Corynebacterium glutamicum strain.

In some embodiments, recombinant host cells comprise heterologous nucleic acids encoding an aspartic acid pathway and/or a β-alanine pathway, and further comprise genetic disruptions to decrease or eliminate expression of one, more or all succinate dehydrogenase subunits. In many embodiments, the succinate dehydrogenase subunit is selected from the group comprising the C. glutamicum SDHA (SEQ ID NO: 10), the C. glutamicum SDHB (SEQ ID NO: 11), and the C. glutamicum SDHC (SEQ ID NO: 2). In some embodiments, recombinant host cells comprise one or more genetic disruptions of a succinate dehydrogenase subunit homolog with least 40%, at least 50%, at least 60%, at least 70%, at least 80%, at least 90%, at least 95%, or more than 95% homology when compared to SEQ ID NO: 10, SEQ ID NO: 11, or SEQ ID NO: 2.

In some embodiments, recombinant host cells comprising heterologous nucleic acids encoding aspartic acid and/or β-alanine pathway enzymes, and genetic disruption of one or more native succinate dehydrogenase subunit homologs will further comprise a succinate byproduct titer of 3 g/l or less, preferably 1 g/l or less, and most preferably 0.5 g/l or less. In certain embodiments, succinate byproduct yield (i.e., percentage of g of byproduct/g of substrate at the end of fermentation) is 10% or less, 5% or less, 2.5% or less, and preferably, 1% or less.

In some embodiments, recombinant host cells comprising heterologous nucleic acids encoding aspartic acid pathway enzymes and genetic disruption of one or more native succinate dehydrogenase subunit homologs will further comprise higher aspartate yield, titer, and/or productivity than cells lacking genetic disruption of the one or more native succinate dehydrogenase subunit homologs. In some embodiments, recombinant host cells comprising heterologous nucleic acids encoding β-alanine pathway enzymes and genetic disruption of one or more native succinate dehydrogenase subunit homologs will further comprise higher β-alanine yield, titer, and/or productivity than cells lacking genetic disruption of the one or more native succinate dehydrogenase subunit homologs.

The construction of recombinant host cells with genetic disruption of succinate dehydrogenase are described below in Examples 2 and 3. The titers for lactate, succinate and aspartic acid of these recombinant host cells are described below in Examples 5 and 8.

### 2.6.3 Decreasing or Eliminating Expression of Alanine Transaminase

Alanine transaminase (EC #2.6.1.2) converts the aspartic acid/β-alanine pathway intermediate pyruvate to L-alanine with concomitant conversion of L-glutamate to 2-oxoglutarate (Table 2). Thus, the expression of endogenous alanine transaminase can decrease anaerobic production of aspartic acid and/or β-alanine. Any enzyme is suitable for use in accordance with the invention so long as the enzyme is capable of catalyzing said alanine transaminase reaction. Genetic disruption of native nucleic acids that encode alanine transaminase is useful for increasing aspartic acid and/or β-alanine titers, yields, and/or productivities. In some embodiments, the recombinant host cell is a Corynebacterium glutamicum strain. In some embodiments, recombinant host cells comprise heterologous nucleic acids encoding an aspartic acid pathway and/or a β-alanine pathway, and further comprise genetic disruptions to decrease or eliminate expression of alanine transaminase or an alanine transaminase homolog.

In some embodiments, recombinant host cells comprising heterologous nucleic acids encoding aspartic acid and/or β-alanine pathway enzymes, and genetic disruption of a native alanine transaminase homolog will further comprise a L-alanine byproduct titer (i.e., g of byproduct/liter of fermentation volume at the end of fermentation) of 10 g/l or less, preferably 5 g/l or less, and most preferably 2.5 g/l or less. In certain embodiments, L-alanine byproduct yield (i.e., percentage of g of byproduct/g of substrate at the end of fermentation) is 10% or less, 5% or less, 2.5% or less, and preferably, 1% or less.

In some embodiments, recombinant host cells comprising heterologous nucleic acids encoding aspartic acid pathway enzymes and genetic disruption of a native alanine transaminase homolog will further comprise higher aspartate yield, titer, and/or productivity than cells lacking genetic disruption of an alanine transaminase homolog. In some embodiments, recombinant host cells comprising heterologous nucleic acids encoding β-alanine pathway enzymes and genetic disruption of a native alanine transaminse homolog will further comprise higher β-alanine yield, titer, and/or productivity than cells lacking genetic disruption of an alanine transaminase homolog.

### 2.6.4 Decreasing or Eliminating Expression of Malate Dehydrogenase

Malate dehydrogenase (EC #1.1.1.37) catalyzes reduction of the aspartic acid/β-alanine pathway intermediate oxaloacetate to malate with concomitant oxidation of NADH to NAD⁺ (Table 2). Thus, the expression of endogenous malate dehydrogenase can decrease anaerobic production of aspartic acid and/or β-alanine both by drawing oxaloacetate out of the aspartic acid/β-alanine pathway and consuming the NADH necessary for reduction of oxaloacetate to aspartic acid. Any enzyme is suitable for use in accordance with the invention so long as the enzyme is capable of catalyzing said malate dehydrogenase reaction. In some embodiments, the malate dehydrogenase has higher specificity for NADH than NADPH. Genetic disruption of native nucleic acids that encode malate dehydrogenase is useful for increasing aspartic acid and/or β-alanine titers, yields, and/or productivities. In some embodiments, the recombinant host cell is a Corynebacterium glutamicum strain. In some embodiments, recombinant host cells comprise heterologous nucleic acids encoding an aspartic acid pathway and/or a β-alanine pathway, and further comprise genetic disruptions to decrease or eliminate expression of malate dehydrogenase.

In some embodiments, the malate dehydrogenase is the C. glutamicum malate dehydrogenase UniProt ID: Q8NN33 (SEQ ID NO: 8). In some embodiments, recombinant host cells comprise genetic disruptions of a homologous malate dehydrogenase gene with least 40%, at least 50%, at least 60%, at least 70%, at least 80%, at least 90%, at least 95%, or more than 95% homology when compared to SEQ ID NO: 34.

In some embodiments, recombinant host cells comprising heterologous nucleic acids encoding aspartic acid and/or β-alanine pathway enzymes, and genetic disruption of a native malate dehydrogenase homolog will further comprise a malate byproduct titer (i.e., g of byproduct/liter of fermentation volume at the end of fermentation) of 10 g/l or less, preferably 5 g/l or less, and most preferably 2.5 g/l or less. In certain embodiments, malate byproduct yield (i.e., percentage of g of byproduct/g of substrate at the end of fermentation) is 10% or less, 5% or less, 2.5% or less, and preferably, 1% or less.

In some embodiments, recombinant host cells comprising heterologous nucleic acids encoding aspartic acid pathway enzymes and genetic disruption of a native malate dehydrogenase homolog will further comprise higher aspartate yield, titer, and/or productivity than cells lacking genetic disruption of a malate dehydrogenase homolog. In some embodiments, recombinant host cells comprising heterologous nucleic acids encoding β-alanine pathway enzymes and genetic disruption of a native malate dehydrogenase homolog will further comprise higher β-alanine yield, titer, and/or productivity than cells lacking genetic disruption of a malate dehydrogenase homolog.

### 2.6.5 Decreasing or Eliminating Expression of Alanine Dehydrogenase

Alanine dehydrogenase (EC #1.4.1.1) catalyzes the conversion of one molecule of pyruvate (a product of glycolysis and a substrate of the aspartic acid and the β-alanine pathways of the present disclosure), one molecule of NH₃, one molecule of NADH and one H⁺ to one molecule of L-alanine, one molecule of water and one molecule of NAD⁺ (Table 2). Thus, the expression of endogenous alanine dehydrogenase can decrease anaerobic production of aspartic acid and/or β-alanine according to the present disclosure both by drawing pyruvate out of the aspartic acid/β-alanine pathway and consuming the NADH necessary for reduction of oxaloacetate to aspartic acid in the aspartic acid/β-alanine pathway. Genetic disruption of native nucleic acids that encode alanine dehydrogenase is useful for increasing aspartic acid and/or β-alanine titers, yields, and/or productivities. In some embodiments, the recombinant host cell is a Corynebacterium glutamicum strain. In some embodiments, recombinant host cells comprise heterologous nucleic acids encoding an aspartic acid pathway and/or a β-alanine pathway, and further comprise genetic disruptions to decrease or eliminate expression of alanine dehydrogenase or an alanine dehydrogenase homolog.

In some embodiments, recombinant host cells comprising heterologous nucleic acids encoding aspartic acid and/or β-alanine pathway enzymes, and genetic disruption of a native alanine dehydrogenase homolog will further comprise a L-alanine byproduct titer (i.e., g of byproduct/liter of fermentation volume at the end of fermentation) of 10 g/l or less, preferably 5 g/l or less, and most preferably 2.5 g/l or less. In certain embodiments, L-alanine byproduct yield (i.e., percentage of g of byproduct/g of substrate at the end of fermentation) is 10% or less, 5% or less, 2.5% or less, and preferably, 1% or less.

In some embodiments, recombinant host cells comprising heterologous nucleic acids encoding aspartic acid pathway enzymes and genetic disruption of a native alanine dehydrogenase homolog will further comprise higher aspartate yield, titer, and/or productivity than cells lacking genetic disruption of an alanine dehydrogenase homolog. In some embodiments, recombinant host cells comprising heterologous nucleic acids encoding β-alanine pathway enzymes and genetic disruption of a native alanine dehydrogenase homolog will further comprise higher β-alanine yield, titer, and/or productivity than cells lacking genetic disruption of an alanine dehydrogenase homolog.

2.6.6 Decreasing or Eliminating Expression of More than One Byproduct Pathway Enzyme for a Synergistic Effect

In some embodiments of the present disclosure, recombinant host cells comprise decreased or eliminated expression of more than one byproduct pathway enzyme. In these embodiments, the recombinant host cells further comprise higher aspartate or β-alanine titer, yield and/or productivity than recombinant host cells that comprise decrease or eliminated expression of only one byproduct pathway enzyme. In some of these embodiments, the recombinant host cells comprise genetic disruptions in some or all of the genes encoding enzymes listed in Table 2. In some embodiments, recombinant host cells comprise decreased or eliminated byproduct accumulation wherein the byproducts are formed through the activity of one, some or all of the enzymes listed in Table 2. In some embodiments, recombinant host cells comprise decreased or eliminated expression of more than one pyruvate-utilizing enzyme. In some embodiments, recombinant host cells comprise decreased or eliminated expression of more than one aspartate, aspartic acid and/or β-alanine-utilizing enzyme. In some embodiments, recombinant host cells comprise inability to metabolize aspartate, aspartic acid and/or β-alanine. In some embodiments, recombinant host cells comprise genetic modifications that reduce the ability of the host cells to metabolize the aspartate or aspartic acid except via the β-alanine pathway. In some embodiments, recombinant host cells comprise genetic modifications that decrease the ability of the host cells to metabolize pyruvate except via the aspartic acid and/or β-alanine pathway. In a particular embodiment, recombinant host cells comprise decrease or eliminated expression of a lactate dehydrogenase homolog and one or more succinate dehydrogenase subunit homologs.

In some embodiments, recombinant host cells which comprise heterologous nucleic acids encoding aspartic acid and/or β-alanine pathway enzymes will further comprise a lactate byproduct titer (i.e., g of byproduct/liter of fermentation volume at the end of fermentation) of 0.5 g/l to 10 g/l or more, and a succinate byproduct titer of 3 g/l or more. In these embodiments, it is beneficial to decrease or eliminate expression of both lactate dehydrogenase and succinate dehydrogenase to decrease lactate and succinate byproduct titers, thereby preventing carbon flux from leaving the aspartic acid/β-alanine pathways.

In some embodiments, recombinant host cells comprising heterologous nucleic acids encoding aspartic acid and/or β-alanine pathway enzymes, and genetic disruption of lactate dehydrogenase and succinate dehydrogenase will further comprise a lactate byproduct titer of 0.5 g/l or less and a succinate byproduct titer of 0.5 g/l or less.

In some embodiments, recombinant host cells comprising heterologous nucleic acids encoding aspartic acid and/or β-alanine pathway enzymes, and genetic disruption of a native lactate dehydrogenase homolog and one or more succinate dehydrogenase subunit homologs will further comprise higher aspartate yield, titer, and/or productivity than recombinant host cells with only genetic disruption in either the native lactate dehydrogenase homolog and the one or more succinate dehydrogenase subunit homologs. In some embodiments, recombinant host cells comprising heterologous nucleic acids encoding aspartic acid and/or β-alanine pathway enzymes, and genetic disruption of a native lactate dehydrogenase homolog and one or more succinate dehydrogenase subunit homologs will further comprise higher β-alanine yield, titer, and/or productivity than recombinant host cells with only genetic disruption in either the native lactate dehydrogenase homolog and the one or more succinate dehydrogenase subunit homologs.

The construction of recombinant host cells with genetic disruption of both lactate dehydrogenase and succinate dehydrogenase are described below in Example 3. The titers for lactate, succinate and aspartic acid of these recombinant host cells are described below in Examples 4 and 8.

### 2.6.7 Decreasing or Eliminating Expression of Acetate Kinase and Phosphate Acetyltransferase

In some embodiments, it is beneficial to decrease or eliminate expression of acetate kinase and phosphate acetyltransferase to decrease acetate byproduct titer, thereby preventing carbon flux from leaving the aspartic acid pathway and/or β-alanine pathway to acetate production.

Acetate kinase (abbv. AckA; EC #2.7.2.1) catalyzes the conversion of acetate and ATP to acetyl phosphate and ADP. Any enzyme is suitable for use in accordance with the invention so long as the enzyme is capable of catalyzing said AckA reaction. Genetic disruption of native nucleic acids that encode AckA is useful for increasing aspartic acid and/or β-alanine titers, yields, and/or productivities. In some embodiments, the recombinant host cell is a Corynebacterium glutamicum strain wherein the Corynebacterium glutamicum AckA is UniProt ID: P77845.

Phosphate acetyltransferase (abbv. Pta; EC #2.3.1.8) catalyzes the conversion of acetyl-CoA and phosphate to acetyl phosphate and CoA. Any enzyme is suitable for use in accordance with the invention so long as the enzyme is capable of catalyzing said Pta reaction. Genetic disruption of native nucleic acids that encode Pta is useful for increasing aspartic acid and/or β-alanine titers, yields, and/or productivities. In some embodiments, the recombinant host cell is a Corynebacterium glutamicum strain wherein the Corynebacterium glutamicum Pta is UniProt ID: P77844.

In some embodiments, recombinant host cells comprise heterologous nucleic acids encoding an aspartic acid pathway and/or a β-alanine pathway, and further comprise genetic disruptions to decrease or eliminate expression of AckA and/or Pta. In some embodiments, the AckA is the C. glutamicum AckA UniProt ID: P77845. In some embodiments, recombinant host cells comprise genetic disruptions of a homologous ACKA gene with least 40%, at least 50%, at least 60%, at least 70%, at least 80%, at least 90%, at least 95%, or more than 95% homology when compared to UniProt ID: P77845. In some embodiments, the Pta is the C. glutamicum Pta UniProt ID: P77844. In some embodiments, recombinant host cells comprise genetic disruptions of a homologous PTA gene with least 40%, at least 50%, at least 60%, at least 70%, at least 80%, at least 90%, at least 95%, or more than 95% homology when compared to UniProt ID: P77844.

In some embodiments, recombinant host cells comprising heterologous nucleic acids encoding aspartic acid and/or β-alanine pathway enzymes, and genetic disruption of a native AckA and/or Pta homolog will further comprise an acetate byproduct titer of 10 g/l or less, preferably 1 g/l or less, and most preferably 0.5 g/l or less. In certain embodiments, acetate byproduct yield (i.e., percentage of g of byproduct/g of substrate at the end of fermentation) is 10% or less, 5% or less, 2.5% or less, and preferably, 1% or less.

In some embodiments, recombinant host cells comprising heterologous nucleic acids encoding aspartic acid pathway enzymes and genetic disruption of a native AckA homolog and/or a Pta dehydrogenase homolog will further comprise higher aspartate yield, titer, and/or productivity than cells lacking said genetic disruption. In some embodiments, recombinant host cells comprising heterologous nucleic acids encoding β-alanine pathway enzymes and genetic disruption of a native AckA homolog and/or a native Pta homolog will further comprise higher β-alanine yield, titer, and/or productivity than cells lacking said genetic disruption.

The construction of recombinant host cells comprising a genetically disrupted lactate dehydrogenase, succinate dehydrogenase, AckA and/or Pta are described below in Examples 1, 3 and 6. The titers for lactate, succinate and aspartic acid of these recombinant host cells are described below in Examples 5 and 8.

### 2.7 Genetic Engineering

Expression of aspartic acid and/or β-alanine pathway enzymes is achieved by transforming host cells with exogenous nucleic acids encoding aspartic acid and/or β-alanine pathway enzymes, producing recombinant host cells of the present disclosure. The same is true for expression of ancillary proteins. Any method can be used to introduce exogenous nucleic acids into a host cell to produce a recombinant host cell of the present disclosure. Many such methods are known to practitioners in the art. Some examples include electroporation, chemical transformation, and conjugation. Some examples include electroporation, chemical transformation, and conjugation. After exogenous nucleic acids enter the host cell, nucleic acids may integrate in to the cell genome via homologous recombination.

Recombinant host cells of the present disclosure may comprise one or more exogenous nucleic acid molecules/elements, as well as single or multiple copies of a particular exogenous nucleic acid molecule/element as described herein. These molecules/elements comprise transcriptional promoters, transcriptional terminators, protein coding regions, open reading frames, regulatory sites, flanking sequences for homologous recombination, and intergenic sequences.

Exogenous nucleic acids can be maintained by recombinant host cells in various ways. In some embodiments, exogenous nucleic acids are integrated into the host cell genome. In other embodiments, exogenous nucleic acids are maintained in an episomal state that can be propagated, either stably or transiently, to daughter cells. Exogenous nucleic acids may comprise selectable markers to ensure propagation. In some embodiments, the exogenous nucleic acids are maintained in recombinant host cells with selectable markers. In some embodiments, the selectable markers are removed and exogenous nucleic acids are maintained in a recombinant host cell strain without selection. In some embodiments, removal of selectable markers is advantageous for downstream processing and purification of the fermentation product.

In some embodiments, endogenous nucleic acids (i.e., genomic or chromosomal elements of a host cell), are genetically disrupted to alter, mutate, modify, modulate, disrupt, enhance, remove, or inactivate a gene product. In some embodiments, genetic disruptions alter expression or activity of proteins native to a host cell. In some embodiments, genetic disruptions circumvent unwanted byproduct formation or byproduct accumulation. Genetic disruptions occur according to the principle of homologous recombination via methods well known in the art. Disrupted endogenous nucleic acids can comprise open reading frames as well as genetic material that is not translated into protein. In some embodiments, one or more marker genes replace deleted genes by homologous recombination. In some of these embodiments, the one or more marker genes are later removed from the chromosome using techniques known to practitioners in the art.

### Section 3. Methods of Producing Aspartic Acid and/or β-Alanine with Recombinant Host Cells

Methods are provided herein for producing aspartic acid and/or β-alanine from recombinant host cells of the present disclosure. In certain embodiments, the methods comprise the steps of: (1) culturing recombinant host cells as provided by the present disclosure in a fermentation broth containing at least one carbon source and one nitrogen source under conditions such that aspartic acid and/or β-alanine is produced; and (2) recovering the aspartic acid and/or β-alanine from the fermentation broth.

As described above in section 2.5.3, one molecule of CO₂ is fixed with the conversion of each molecule of glucose to aspartate or β-alanine (FIG. 1). An abundant pool of HCO₃⁻ helps the oxaloacetate-forming enzyme reactions (FIG. 1 and Table 1) move forward and prevents these steps in the aspartic acid and β-alanine pathways from becoming a bottleneck of the pathways. Thus, in some embodiments, the methods further comprise culturing recombinant host cells in a way that results in increased CO₂ uptake by the recombinant host cells. In some embodiments, the methods comprise culturing recombinant host cells with an exogenous source of CO₂ or culturing recombinant host cells under a CO₂ partial pressure that is higher than atmospheric CO₂ partial pressure.

3.1 Fermentative Production of Aspartic Acid and/or β-Alanine by Recombinant Host Cells

Any of the recombinant host cells of the present disclosure can be cultured to produce and/or secrete aspartic acid and/or β-alanine.

Materials and methods for the maintenance and growth of microbes, as well as fermentation conditions, are well known to practitioners of ordinary skill in the art. It is understood that consideration must be given to appropriate culture medium, pH, temperature, revival of frozen stocks, growth of seed cultures and seed trains, and requirements for aerobic, microaerobic, or anaerobic conditions, depending on the specific requirements of the host cells, the fermentation, and process flows.

The methods of producing aspartic acid and/or β-alanine provided herein may be performed in a suitable fermentation broth in a suitable bioreactor such as a fermentation vessel, including but not limited to a culture plate, a flask, or a fermenter. Further, the methods can be performed at any scale of fermentation known in the art to support microbial production of small-molecules on an industrial scale. Any suitable fermenter may be used including a stirred tank fermenter, an airlift fermenter, a bubble column fermenter, a fixed bed bioreactor, or any combination thereof.

In some embodiments of the present disclosure, the fermentation broth is any fermentation broth in which a recombinant host cell capable of producing aspartic acid and/or β-alanine according to the present disclosure, and can subsist (i.e., maintain growth, viability, and/or catabolize glucose or other carbon source). In some embodiments, the fermentation broth is an aqueous medium comprising assimilable carbon, nitrogen, and phosphate sources. Such a medium can also include appropriate salts, minerals, metals, and other nutrients. In some embodiments, the carbon source and each of the essential cell nutrients are provided to the fermentation broth incrementally or continuously, and each essential cell nutrient is maintained at essentially the minimum level required for efficient assimilation by growing cells. Exemplary cell growth procedures include batch fermentation, fed-batch fermentation with batch separation, fed-batch fermentation with continuous separation, and continuous fermentation with continuous separation. These procedures are well known to practitioners of ordinary skill in the art.

In some embodiments of the present disclosure, the handling and culturing of recombinant host cells to produce aspartic acid and/or β-alanine may be divided up into phases, such as growth phase, production phase, and/or recovery phase. The following paragraphs provide examples of features or purposes that may relate to these different phases. One skilled in the art will recognize that these features or purposes may vary based on the recombinant host cells used, the desired aspartic acid and/or β-alanine yield, titer, and/or productivity, or other factors. While it may be beneficial in some embodiments for the aspartic acid and/or β-alanine pathway enzymes, ancillary proteins and/or endogenous host cell proteins to be constitutively expressed, in other embodiments, it may be preferable to selectively express or repress any or all of the aforementioned proteins.

During growth phase, recombinant host cells may be cultured to focus on growing cell biomass by utilizing the carbon source provided. In many embodiments, the growth phase is performed under aerobic conditions. In some embodiments, the expression of aspartic acid and/or β-alanine pathway enzymes and/or ancillary proteins is repressed or uninduced. In some embodiments, no appreciable amount of aspartic acid and/or β-alanine is made. In some embodiments, proteins that contribute to cell growth and/or cellular processes may be selectively expressed.

During production phase, however, recombinant host cells may be cultured to stop producing cell biomass and to focus on aspartic acid and/or β-alanine biosynthesis by utilizing the carbon source provided. In many embodiments, the production phase is performed under substantially anaerobic, microanaerobic, or oxygen-limited conditions, wherein the recombinant host cells stop growing and directs resources through the aspartic acid or β-alanine pathways of the present disclosure as a means to consume glucose and recycle NAD(P)H. In some embodiments, aspartic acid and/or β-alanine pathway enzymes, and/or ancillary proteins may be selectively expressed during production to generate high product titers, yields and productivities. The production phase is synonymous with fermentation, fermentation run or fermentation phase.

In some embodiments, the growth and production phases take place at the same time. In other embodiments, the growth and production phases are separate. While in some embodiments, product is made exclusively during production phase, in other embodiments some product is made during growth phase before production phase begins.

The recovery phase marks the end of the production phase, during which cellular biomass is separated from fermentation broth and aspartic acid and/or β-alanine is purified from fermentation broth. Those skilled in the art will recognize that in some fermentation process, e.g., fill-draw and continuous fermentations, there may be multiple recovery phases where fermentation broth containing biomass and aspartic acid and/or β-alanine are removed from the fermentation system. The draws of fermentation broth may be processed independently or may be stored, pooled, and processed together. In other fermentation processes, e.g., batch and fed-batch fermentations, there may only be a single recovery phase.

Fermentation procedures are particularly useful for the biosynthetic production of commercial aspartic acid and/or β-alanine. It is understood by practitioners of ordinary skill in the art that fermentation procedures can be scaled up for manufacturing aspartic acid and/or β-alanine and exemplary fermentation procedures include, for example, fed-batch fermentation and batch product separation; fed-batch fermentation and continuous product separation; batch fermentation and batch product separation; and continuous fermentation and continuous product separation.

### 3.1.1 Carbon Source

The carbon source provided to the fermentation can be any carbon source that can be fermented by recombinant host cells. Suitable carbon sources include, but are not limited to, monosaccharides, disaccharides, polysaccharides, glycerol, acetate, ethanol, methanol, methane, or one or more combinations thereof. Exemplary monosaccharides suitable for use in accordance to the methods of the present disclosure include, but are not limited to, dextrose (glucose), fructose, galactose, xylose, arabinose, and any combination thereof. Exemplary disaccharides suitable for use in accordance to the methods of the present disclosure include, but are not limited to, sucrose, lactose, maltose, trehalose, cellobiose, and any combination thereof. Exemplary polysaccharides suitable for use in accordance to the methods of the present disclosure include, but are not limited to, starch, glycogen, cellulose, and combinations thereof. In some embodiments, the carbon source is dextrose. In other embodiments, the carbon source is sucrose. In some embodiments, mixtures of some or all the aforementioned carbon sources can be used in fermentation.

3.1.2 pH

The pH of the fermentation can significantly affect aspartic acid production by influencing CO₂ solubility in the fermentation. The PYC, PCK, and PPC enzymes of the aspartic acid and β-alanine pathways each utilize a molecule of HCO₃⁻ for the production of every molecule of oxaloacetate (Table 1). Within the pH range of about 6.5 to about 8.5, aspartic acid titer climbs with decreasing pH. The pH of the fermentation broth can be controlled by the addition of acid or base to the culture medium. Specifically, the pH during fermentation is maintained in the range of 6-8, and more preferably in the range of 6.5-7.5. Non-limiting examples of suitable acids used to control fermentation pH include aspartic acid, acetic acid, hydrochloric acid, and sulfuric acid. Non-limiting examples of suitable bases used to control fermentation pH include sodium bicarbonate (NaHCO₃), sodium hydroxide (NaOH), potassium bicarbonate (KHCO₃), potassium hydroxide (KOH), calcium hydroxide (Ca(OH)₂), calcium carbonate (CaCO₃), ammonia, ammonium hydroxide, and diammonium phosphate. In some embodiments, a concentrated acid or concentrated base is used to limit dilution of the fermentation broth. In some embodiments, the base is ammonium hydroxide. In some embodiments, the base is sodium hydroxide.

### 3.1.3 Temperature

In general, the temperature of the fermentation broth can be any temperature suitable for growth of the recombinant host cells and/or production of aspartic acid and/or β-alanine. Preferably, during aspartic acid and/or β-alanine production, the fermentation broth is maintained within a temperature range of from about 20° C. to about 45° C., and more preferably in the range of from about 30° C. to about 42° C.

In embodiments where the recombinant host cell is able to tolerate higher temperatures without growth defects, higher temperatures increase enzyme kinetics of the aspartic acid and/or β-alanine pathway, thus improving aspartic acid productivity. In embodiments where C. glutamicum is the recombinant host cell, the ATCC recommended growth temperature is 30° C. to 33° C. If C. glutamicum is able to tolerate higher temperatures without growth defects, such as a temperature of about 37° C., the fermentation temperature is maintained at 37° C.

In some embodiments, the growth temperature is different from the production temperature. In some embodiments, the growth temperature is lower than the production temperature. In some embodiments, the growth temperature is 30° C. to 33° C. and the production temperature is 37° C. In these embodiments, glucose consumption rate is improved by 5-20%, and aspartic acid productivity is improved by 10-30%.

### 3.1.4 Oxygen/Aeration

The present disclosure provides methods to achieve high aspartic acid and/or β-alanine yields, titers, and/or productivities wherein recombinant host cells are under aerobic conditions during growth phase, and anaerobic or microaerobic conditions during production phase. Buildup of oxidized cofactor NAD(P)⁺ is inherent to the aspartic acid and β-alanine pathways of the present disclosure at the step catalyzed by AspDH (FIG. 1 and Table 1; Section 2.2.2.1). Reduction of NAD(P)⁺ back to NAD(P)H can help ensure pathway flux is not impeded by NAD(P)H depletion. During production phase under anaerobic or microaerobic conditions, recombinant host cells reduce NAD(P)⁺ through the activity of GAPDH in glycolysis. Thus, recombinant host cells are required to maintain glycolysis during production phase, as well as keep carbon flux from leaving the aspartic acid and β-alanine pathways, thereby linking high glucose consumption to high aspartic acid/β-alanine yields, titers, and/or productivities.

During production phase, aeration and agitation conditions are selected to produce an oxygen transfer rate (OTR; rate of dissolution of dissolved oxygen in a fermentation medium) that results in aspartic acid production. In various embodiments, fermentation conditions are selected such that no oxygen is transferred (i.e., OTR of 0 mmol/l/hr). In some embodiments, fermentation conditions are selected to produce an OTR of less than 1 mmol/l/hr. In some embodiments, fermentation conditions are selected to produce an OTR of less than 5 mmol/l/hr. In some embodiments, fermentation conditions are selected to produce an OTR of less than 10 mmol/l/hr. OTR as used herein refers to the volumetric rate at which oxygen is consumed during the fermentation. Inlet and outlet oxygen concentrations can be measured by exhaust gas analysis, for example by mass spectrometers. OTR can be calculated by one of ordinary skill in the art using the Direct Method described in Bioreaction Engineering Principles 3^{rd} Edition, 2011, Spring Science+Business Media, p. 449.

In some embodiments, recombinant host cells are cultured in a BD Biosciences GasPak™ EZ container system to maintain an anaerobic environment. The BD Biosciences GasPak™ EZ container system was used according to manufacturer recommendations.

### 3.1.5 Carbon Dioxide Supplementation

In the aspartic acid and β-alanine pathways of the present disclosure, one molecule of CO₂, after conversion to HCO₃⁻ in recombinant host cells, is utilized with the conversion of each molecule of glucose to aspartate or β-alanine (FIG. 1; section 2.5.3). PYC, PCK, and PPC of the aspartic acid and β-alanine pathways each utilize a molecule of HCO₃⁻ for the production of every molecule of oxaloacetate (Table 1). Under anaerobic or microaerobic production conditions, little to no CO₂ is produced by recombinant host cells, which may lead to insufficient CO₂ availability for PYC, PCK and/or PPC, resulting in a decrease in pathway activity. Further, the pH of fermentation medium can influence the interconversion of CO₂ to HCO₃⁻ and the solubility of HCO₃⁻ within recombinant host cells. Thus, while higher concentrations of CO₂ are generally helpful in maintaining aspartic acid and β-alanine pathway flux, it is especially important when pH values are relatively acidic. For example, the HCO₃⁻:CO₂ ratio in a pH range of 5-9 is higher when compared the HCO₃⁻:CO₂ ratio in a pH range of 1-4. Therefore, in embodiments wherein pH in the fermentation medium is relatively acidic during production phase, a greater amount of exogenous CO₂ is supplied to maintain high HCO₃⁻ availability in recombinant host cells.

In some of embodiments, the exogenous supply of CO₂ is a gaseous CO₂. In some embodiments, the partial pressure of CO₂ in production phase is higher than the partial pressure of CO₂ in growth phase. In some embodiments, the exogenous supply of CO₂ is a salt, such as calcium carbonate or sodium bicarbonate.

In some embodiments, recombinant host cells are cultured in a BD Biosciences GasPak™ EZ container system. In other embodiments, recombinant host cells are cultured in air-tight 96-deep well plates with a gas mixture comprising N2 and CO₂. In various embodiments, the gas mixture is supplied at a flow rate of at least 0.2 l/min. In various embodiments, the concentration of CO₂ in the gas mixture is at least 10%, at least 20%, or at least 30%.

### 3.1.6 Yields and Titers

A high yield of aspartic acid and/or β-alanine from the provided carbon source(s) is desirable to decrease the production cost. As used herein, yield is calculated as the percentage of the mass of carbon source catabolized by recombinant host cells of the present disclosure and used to produce aspartic acid and/or β-alanine. In some cases, only a fraction of the carbon source provided to a fermentation is catabolized by the cells, and the remainder is found unconsumed in the fermentation broth or is consumed by contaminating microbes in the fermentation. Thus, it is important to ensure that fermentation is both substantially pure of contaminating microbes and that the concentration of unconsumed carbon source at the completion of the fermentation is measured. For example, if 100 grams of glucose is fed into the fermentation, and at the end of the fermentation 25 grams of aspartic acid is produced and there remains 10 grams of glucose, the aspartic acid yield is 27.7% (i.e., 25 grams aspartic acid from 90 grams glucose). In certain embodiments of the methods provided herein, the final yield of aspartic acid and/or β-alanine on the carbon source is at least 10%, at least 20%, at least 30%, at least 35%, at least 40%, at least 50%, at least 60%, at least 70%, at least 80%, or greater than 80%. In certain embodiments, the recombinant host cells provided herein are capable of producing at least 70%, at least 75%, or greater than 80% by weight of carbon source to aspartic acid and/or β-alanine.

In addition to yield, the titer (or concentration), of aspartic acid and/or β-alanine produced in the fermentation is another important metric for production. Assuming all other metrics are equal, a higher titer is preferred to a lower titer. Generally speaking, titer is provided as grams of product (e.g., aspartic acid and/or β-alanine) per liter of fermentation broth (i.e., g/l). In some embodiments, the aspartic acid and/or β-alanine titer is at least 1 g/l, at least 5 g/l, at least 10 g/l, at least 15 g/l, at least 20 g/l, at least 25 g/l, at least 30 g/l, at least 40 g/l, at least 50 g/l, at least 60 g/l, at least 70 g/l, at least 80 g/l, at least 90 g/l, at least 100 g/l, at least 125 g/l, at least 150 g/l, or greater than 150 g/l at some point during the fermentation, and preferably at the conclusion of the fermentation. In some embodiments, the aspartic acid and/or β-alanine titer at the conclusion of the fermentation is greater than 100 g/l. In some embodiments, the aspartic acid and/or β-alanine titer at the conclusion of the fermentation is greater than 125 g/l. In some embodiments, the aspartic acid and/or β-alanine titer at the conclusion of the fermentation is greater than 150 g/l.

Further, productivity, or the rate of product (i.e., aspartic acid and/or β-alanine) formation, is important for decreasing production cost, and, assuming all other metrics are equal a higher productivity is preferred over a lower productivity. Generally speaking, productivity is provided as grams product produced per liter of fermentation broth per hour (i.e., g/l/hr). In some embodiments, aspartic acid and/or β-alanine productivity is at least 0.1 g/l/hr, at least 0.25 g/l/hr, at least 0.5 g/l/hr, at least 0.75 g/l/hr, at least 1.0 g/l/hr, at least 1.25 g/l/hr, at least 1.25 g/l/hr, at least 1.5 g/l/hr, at least 2.0 g/l/hr, at least 3.0 g/l/hr, at least 4.0 g/l/hr, at least 5.0 g/l/hr, at least 6.0 g/l/hr or greater than 6.0 g/l/hr over some time period during the fermentation. In some embodiments, the aspartic acid and/or β-alanine productivity is at least 3 g/l/hr. In some embodiments, the aspartic acid and/or β-alanine productivity is at least 4 g/l/hr. In some embodiments, the aspartic acid and/or β-alanine productivity is at least 5 g/l/hr.

Practitioners of ordinary skill in the art understand that HPLC is an appropriate method to determine the amount of aspartic acid and/or β-alanine and/or produced, the amount of any byproducts produced (e.g., organic acids and alcohols), the amount of any pathway metabolite or intermediate produced, and the amount of unconsumed glucose left in the fermentation broth. Aliquots of fermentation broth can be isolated for analysis at any time during fermentation, as well as at the end of fermentation. Briefly, molecules in the fermentation broth are first separated by liquid chromatography (LC); then, specific liquid fractions are selected for analysis using an appropriate method of detection (e.g., UV-VIS, refractive index, and/or photodiode array detectors). In some embodiments of the present disclosure, an organic acid salt (e.g., aspartic acid and/or β-alanine) is the fermentative product present in the fermentation broth. Practitioners in the art understand that the salt is acidified before or during HPLC analysis to produce aspartic acid and/or β-alanine. Hence, the acid concentration calculated by HPLC analysis can be used to calculate the salt titer in the fermentation broth by adjusting for difference in molecular weight between the two compounds.

Gas chromatography-mass spectrometry (GC-MS) is also an appropriate method to determine the amount of target product and byproducts, particularly if they are volatile. Samples of fermentation can be isolated any time during and after fermentation and volatile compounds in the headspace can be extracted for analysis. Non-volatile compounds in the fermentation medium (e.g., organic acids) can also be analyzed by GC-MS after derivatization (i.e., chemical alteration) for detection by GC-MS. Non-volatile compounds can also be extracted from fermentation medium by sufficiently increasing the temperature of the fermentation medium, causing non-volatile compounds to transition into gas phase for detection by GC-MS. Practitioners in the art understand that molecules are carried by an inert gas carries as they move through a column for separation and then arrive at a detector.

### Section 4. Purification of Aspartic Acid, Aspartate Salts, and β-Alanine

The present disclosure describes the methods for purifying and analyzing fermentation product synthesized by recombinant cells of the present disclosure, wherein the fermentation product comprises aspartic acid, aspartate salts, and/or β-alanine. The methods comprise separating soluble fermentation product from fermentation broth, cells, cell debris and soluble impurities, and isolating the soluble fermentation product. In some examples, the methods may also comprise converting fermentation product from soluble form to insoluble, crystalline form, and isolating the crystalline fermentation product.

At the end of fermentation, the fermentation broth contains fermentation product, in soluble and/or insoluble forms, together with biomass and soluble impurities that include salts, proteins, unconverted sugars, and other impurities including color bodies. Biomass and soluble impurities are removed via a series of purification steps. In certain embodiments of the present disclosure, purification steps may include centrifugation, microfiltration, ultrafiltration, nanofiltration, diafiltration, ion exchange, crystallization, and any combination thereof. In some of these embodiments, ion exchange resins and nanofiltration membranes are used as polishing steps to remove trace amounts of soluble impurities, unconverted sugars and color bodies.

4.1 Removal of cells and cell debris

In some embodiments, the process of purifying fermentation product (i.e., aspartic acid, aspartate salts, and/or β-alanine) comprises a step of separating a liquid fraction containing fermentation product from a solid fraction that contains cells and cell debris. For this separation, any amount of fermentation broth can be processed, including the entirety of the fermentation broth. One skilled in the art will recognized the amount of fermentation broth processed can depend on the type of fermentation process used, such as batch or continuous fermentation processes. In various embodiments, removal of cells and cell debris can be accomplished, for example, via centrifugation using specific g-forces and residence times, and/or filtration using molecular weight cutoffs that are suitable for efficiently separating the liquid fraction containing fermentation product from the solid fraction that contains cells and cell debris. In some embodiments, removal of cells and cell debris is repeated at least once at one or in more than one step in the methods provided herein.

In some embodiments, centrifugation is used to provide a liquid fraction comprising fermentation product that is substantially free of cells. Many types of centrifuges useful for the removal of cells and solids from fermentation broth are known to those skilled in the art, including disc-stack and decanter centrifuges. Centrifuges are well suited for separating solids with a particle size of between 0.5 μm to 500 μm and density greater than that of the liquid phase (ca. 1.0 g/ml). Yeast cells, as a non-limiting example of a fermentation product-producing microbe, typically have a particle size between 4-6 μm and a density of around 1.1 g/ml; therefore, centrifugation is well suited for removing yeast cells from fermentation broth.

In some embodiments, a disc-stack centrifuge is used to provide a liquid fraction comprising fermentation product that substantially free of cells. A disc stack centrifuge separates solids, which are discharged intermittently during operation, from liquids, typically in a continuous process. A disc-stack centrifuge is well suited for separating soft, non-abrasive solids, including cells. In some embodiments, a decanter centrifuge is used to provide a liquid fraction comprising fermentation product that is substantially free of cells. A decanter centrifuge can typically process larger amounts of solids and is often preferred over a disc-stack centrifuge for processing fermentation broth when the cell mass and other solids exceeds about 3% w/w.

Other methods can be used in addition to, or alone, with the above centrifugation processes. For example, microfiltration is also an effective means to remove cells from fermentation broth. Microfiltration includes filtering the fermentation broth through a membrane having pore sizes from about 0.5 μm to about 5 μm. In some embodiments, microfiltration is used to provide a liquid fraction comprising fermentation product that is substantially free of cells.

In some embodiments, cells removed by centrifugation and/or microfiltration are recycled back into the fermentation. One skilled in the art will recognize recycling cells back into the fermentation can increase fermentation product yield since less carbon source (e.g., glucose) must be used to generate new cells. Additionally, recycling cells back into the fermentation can also increase fermentation product productivity since the concentration of cells producing aspartic acid and/or β-alanine in the fermenter can be increased.

While suitable for removing cells, centrifugation and microfiltration are generally not effective at removing cells debris, proteins, DNA and other smaller molecular weight compounds from the fermentation broth. Ultrafiltration is a process similar to microfiltration, but the membrane has pore sizes ranging from about 0.005 μm to 0.1 μm. This pore size equates to a molecular weight cut-off (the size of macromolecule that will be ca. 90% retained by the membrane) from about 1,000 Daltons to about 200,000 Daltons. The ultrafiltration permeate will contain low-molecular weight compounds, including fermentation product and various other soluble salts while the ultrafiltration retentate will contain the majority of residual cell debris, DNA, and proteins. In some embodiments, ultrafiltration is used to provide a liquid fraction comprising aspartic acid and/or β-alanine that is substantially free of cell debris and proteins.

### 4.2 Nanofiltration and Ion Exchange Polishing of Clarified Fermentation Broth Containing Fermentation Product

In some embodiments, nanofiltration is used to separate out certain contaminating salts, sugars, color forming bodies, and other organic compounds present in clarified fermentation broth containing fermentation product (i.e., aspartic acid, aspartate salts, and/or β-alanine). In nanofiltration, the clarified fermentation broth (i.e., the fermentation broth resulting from the combination of centrifugation, microfiltration, and/or ultrafiltration steps described above) is filtered through a membrane having pore sizes ranging from 0.0005 μm to 0.005 μm, equating to a molecular weight cut-off of about 100 Daltons to about 2,000 Daltons. Nanofiltration can be useful for removing divalent and multivalent ions, maltose and other disaccharides (e.g., sucrose), polysaccharides, and other complex molecules with a molecular weight substantially larger than fermentation product (e.g., aspartic acid, aspartate salts, and/or β-alanine). Non-limiting examples of nanofiltration materials include ceramic membranes, metal membranes, polymer membranes, activated carbon, and composite membranes.

In some embodiments, ion exchange is used to remove specific contaminating salts present in clarified fermentation broth containing fermentation product. Ion exchange elements can take the form of resin beads as well as membranes. Frequently, the resins are cast in the form of porous beads. The resins can be cross-linked polymers having active groups in the form of electrically charged sites. At these sites, ions of opposite charge are attracted but may be replaced by other ions depending on their relative concentrations and affinities for the sites. Ion exchangers can be cationic or anionic. Factors that determine the efficiency of a given ion exchange resin include the favorability for a given ion, and the number of active sites available.

Practitioners of ordinary skill in the art understand that a combination of nanofiltration and ion exchange steps can be combined and modified to produce a purified solution of fermentation product.

### 4.3 Acidification of Purified Solution of Fermentation Product

In some embodiments, the methods comprise acidification of purified solution of fermentation product to convert fermentation salt products to aspartic acid. Non-limiting examples of acids that can be used for this acidification step include sulfuric acid, hydrochloric acid, phosphoric acid, and nitric acid. In some embodiments, a concentrated acid is used to limit dilution of the aspartic acid produced.

In some embodiments, the fermentation salt products are aspartate salts. In some embodiments, the aspartate salt is sodium aspartate. In some embodiments, the aspartate salt is ammonium aspartate. In some embodiments, an acid such as sulfuric acid is added to the clarified fermentation broth to convert the aspartate salt to sulfate salt and aspartic acid. In some embodiments, the sulfate salt is sodium sulfate. In some embodiments, the sulfate salt is ammonium sulfate.

### 4.4 Crystallization of Fermentation Product

In some embodiments, the methods comprise a crystallization step to purify aspartic acid and/or β-alanine from the purified solution of fermentation product as described thus far. The crystallization step removes water and water-soluble impurities. In some embodiments of the present disclosure, it is desirable to recover the majority of the aspartic acid and/or β-alanine in the insoluble, crystallized form with a minor fraction of aspartic acid and/or β-alanine remaining in the mother liquor.

In some embodiments, the purified solution of fermentation product comprises aspartate salts and aspartic acid. In some embodiments, the aspartate salt is sodium aspartate. In some embodiments, the aspartate salt is ammonium aspartate. Because the aspartate salts have substantially higher solubility than aspartic acid, aspartic acid can be purified from solution by crystallization. For example, at room temperature, sodium aspartate is soluble in water at greater than 100 g/l and ammonium aspartate is soluble in water at ca. 600 g/l, while aspartic acid is soluble in water at ca. 4.5 g/l. In some embodiments, the aspartic acid is crystallized without additional concentration and/or cooling steps. In some embodiments, one or more concentration steps precede crystallization. The fermentation product in the aqueous fermentation broth is concentrated by one or more steps, wherein the one or more steps comprises centrifuging, heating, cooling, filtering, distilling, evaporating, or any combination thereof.

In some embodiments, the purified solution of fermentation product comprises sulfate salts and aspartic acid. In some embodiments, the sulfate salt is sodium sulfate. In some embodiments, the sulfate salt is ammonium sulfate. Because the sulfate salts have substantially higher solubility than aspartic acid, aspartic acid can be purified from solution by crystallization. For example, at room temperature, sodium sulfate is soluble in water at ca. 20 g/l and ammonium sulfate is soluble in water at ca. 76 g/l, while aspartic acid is soluble in water at ca. 4.5 g/l. In some embodiments, the aspartic acid is crystallized without additional concentration and/or cooling steps.

In some embodiments of the present disclosure, the temperature of the mother liquor is changed to facilitate fermentation product crystallization. In some embodiments, the mother liquor is cooled to a temperature below 20° C. to decrease fermentation product solubility. In some these embodiments, the mother liquor is heated to evaporate excess water.

In some of these embodiments, evaporative crystallization is preferred as it offers a high yield of fermentation product and prevents the formation of stable gels, which may occur if temperature is reduced below the gelling point of concentrated fermentation product solutions. In some of these embodiments, fermentation product crystallization is achieved by combining various heating and cooling steps. In some of these embodiments, supersaturation is achieved by evaporative crystallization wherein the solute is more concentrated in a bulk solvent that is normally possible under given conditions of temperature and pressure; increased supersaturation of fermentation product in the mother liquor causes the fermentation product to crystallize. Non-limiting examples of crystallizers include forced circulation crystallizers, turbulence/draft tube and baffle crystallizers, induced circulation crystallizers and Oslo-type crystallizers.

In some embodiments of the present disclosure, the aforementioned heating step, cooling step and change in pH are combined in various ways to crystallize fermentation product, and modified as needed, as apparent to practitioners skilled in the art.

Fermentation product crystals can be isolated from the mother liquor by any technique apparent to those of skill in the art. In some embodiments of the present disclosure, fermentation product crystals are isolated based on size, weight, density, or combinations thereof. Fermentation product crystal isolation based on size can be accomplished, for example, via filtration, using a filter with a specific particle size cutoff. Fermentation product crystal isolation based on weight or density can be accomplished, for example, via gravitational settling or centrifugation, using, for example, a settler, decanter centrifuge, disc-stack centrifuge, basket centrifuge, or hydrocyclone wherein suitable g-forces and settling or centrifugation times can be determined using methods known in the art. In some embodiments, fermentation product crystals are isolated from the mother liquor via settling for from 30 minutes to 2 hours at a g-force of 1. In other embodiments, aspartic acid, aspartate salt, and/or β-alanine crystals are isolated from the fermentation broth via centrifugation for 20 seconds to 60 seconds at a g-force of from 275 x-g to 1,000 x-g.

Following isolation from the mother liquor, fermentation product crystals are wet with residual mother liquor that coats the crystals. Thus, it is useful to wash the fermentation product crystals with water to remove these trace impurities that may be in the mother liquor. When washing fermentation product crystals, it is important to minimize the dissolution of isolated crystals in the wash water; for this reason, cold wash (around 4° C.) water is generally used. Additionally, it is important to minimize the amount of wash water used to minimize crystal dissolution. In many embodiments, less than 10% w/w wash water is used to wash the fermentation product crystals.

In some embodiments, the methods further comprise the step of removing impurities from fermentation product crystals. Impurities may react with fermentation product crystals and reduce final yields or contribute to fermentation product crystals of lesser purity that limits industrial utility. Non-limiting examples of impurities include acetic acid, succinic acid, malic acid, ethanol, glycerol, citric acid, and propionic acid. In some embodiments, removal of such impurities is accomplished by dissolving the isolated fermentation product crystals into an aqueous solution and recrystallizing the fermentation product. A non-limiting example of dissolving and recrystallizing fermentation product crystals can include dissolving the fermentation product in water and evaporating the resulting aqueous solution (as mentioned above), and finally re-isolating the fermentation product crystals by filtration and/or centrifugation. None, one, or more than one cycle of fermentation product recrystallization may be used so long as the resulting fermentation product are of suitable quality for subsequent esterification. In some embodiments, no fermentation product recrystallizations are performed. In other embodiments, one fermentation product recrystallization is performed. In still further embodiments, more than one fermentation product recrystallization is performed.

In some embodiments of the present disclosure, fermentation product crystals are dewatered using a combination of screening and drying methods apparent to practitioners skilled in the art. In some of these embodiments, crystal dewatering steps comprise centrifugation, belt drying, filtration, application of vacuum, or a combination thereof. In some of these embodiments, vacuum is applied at 20 mm of Hg below atmospheric pressure. Suitable devices for crystal dewatering may include a Horizontal Vacuum Belt Filter (HVBF) or a Rotary Drum Vacuum Filter (RDVF). Fermentation product crystal isolation based on size can be accomplished, for example, via filtration, using, for example, a filter press, candlestick filter, or other industrially used filtration system with appropriate molecular weight cutoff. Fermentation product crystal isolation based on weight or density can be accomplished, for example, via gravitational settling or centrifugation, using, for example, a settler, decanter centrifuge, disc-stack centrifuge, basket centrifuge, or hydrocyclone, wherein suitable g-forces and settling or centrifugation times can be determined using methods known in the art.

In some embodiments of the present disclosure, fermentation products are crystallized in the fermentation broth prior to removal of cells, cell debris, contaminating salts and various soluble impurities. In many of these embodiments, the fermentation product crystals are separated from fermentation broth, cells, cell debris, contaminating salts and various soluble impurities by sedimentation, centrifugation, ultrafiltration, nanofiltration, ion exchange, or any combination thereof.

In some embodiments, the mother liquor that is leftover from crystallization or the supernatant obtained after a crystallization is further treated so that the minor fraction of aspartic acid or the salt thereof remaining in the mother liquor may be isolated. The mother liquor is concentrated by one or more steps, wherein the one or more steps comprises centrifuging, heating, cooling, filtering, distilling, evaporating, or any combination thereof. The heating and cooling steps may include heating to 80° C. and cooling slowly to 20° C. The recovered minor fraction of aspartic acid is crystallized by the addition of an acid, and the resulting crystals are dried by evaporation at room temperature or at an elevated temperature in an oven. Non-limiting examples of acids that can be used are mineral acids, such as sulfuric acid, hydrochloric acid, hydrohalic acids, nitric acid and perchloric acid, and resin-based acids such as polystyrene sulfonic acid. As described above, the aspartic acid crystals are isolated by one or more filtration steps.

### Section 5. Examples

### Media Used in Examples

Brain heart infusion (BHI) medium.

BHI medium comprised beef heart (infusion from 250 g) 5 g/L, calf brains (infusion from 200 g) 12.5 g/L, disodium hydrogen phosphate 2.5 g/L, D(+)-glucose 2 g/L, peptone 10 g/L, and sodium chloride 5 g/L.

Brain Heart Infusion Medium with Kanamycin (BHI+Kan).

BHI+Kan comprised BHI medium and 25 μg/ml kanamycin.

Brain Heart Infusion Medium with Kanamycin (BHI+Kan+Spec).

BHI+Kan+Spec comprised BHI medium, 25 μg/ml kanamycin, and 50 μg/ml spectinomycin.

Brain Heart Infusion Medium with MOPS and Glucose (BHI+MOPS+Glucose).

BHI+MOPS+glucose comprised BHI medium and 50 mM MOPS with pH adjusted with KOH to pH 7.5, and 2% glucose.

Trace Elements, 1000× Stock Solution.

This trace elements solution comprised FeSO₄.7H₂O 10 g/L, MnSO₄.H₂O 10 g/L, ZnSO₄.7H₂O 1 g/L, CuSO₄ 0.2 g/L, and NiCl₂.6H₂O 0.02 g/L.

CGXII Medium.

CGXII comprised MOPS (pH 7.5 with KOH) 0.2 M, urea 0.16M, KH₂PO₄ 7.35 mM, K₂HPO₄ 5.74 mM, MgSO₄.7H₂O 1.01 mM, CaCl₂.2H₂O 0.07 mM, FeSO₄.7H₂O 10 mg/L, biotin 0.2 mg/L, protochatechuic acid 0.2 mM, trace elements solution at a final concentration of 1×, glucose 4% (w/v), and NaHCO₃200 mM.

CGXII Medium with Kanamycin (CGXII+Kan).

CGXII+Kan comprised CGXII and 25 μg/ml kanamycin.

### Example 1: Construction of Recombinant Corynebacterium glutamicum Strain LCG4004 with Eliminated Expression of Lactate Dehydrogenase

Example 1 describes the construction of a lactate dehydrogenase (LDHA) minus C. glutamicum, LCG4004, wherein expression of LDHA in C. glutamicum (abbv. CgLDHA; SEQ ID NO: 1) was eliminated via genetic disruption of the LdhA gene. LCG4004 cells with elimination of CgLDHA expression were unable to convert pyruvate to lactate, thus not depleting the cellular pool of pyruvate that may be available for the aspartic acid/β-alanine pathway. The culturing and analysis of LCG4004 is described below in Examples 4 and 5.

The parent C. glutamicum strain for all recombinant strains described herein is designated LCG4002. CgLDHA was genetically disrupted in LCG4002 using the temperature sensitive-sacB (ts-sacB) markerless deletion methodology described by Okibe et al in Journal of Microbiological Methods 85 (2011) 155-163. Briefly, plasmid pLCSac-LDH\hat{ } was constructed to comprise a ts-sacB gene flanked by an upstream transcriptional promoter and a downstream transcriptional terminator. pLCSac-LDHA further comprised unique upstream (SEQ ID NO: 3) and downstream (SEQ ID NO: 4) homologous regions to C. glutamicum LdhA for homologous recombination at the C. glutamicum LdhA locus. pLCSac-LDHA also comprised a kanamycin resistant gene. Transformation of C. glutamicum with pLCSac-LDHA was carried out according to the two-step process disclosed by Okibe et al, which comprised two temperature selection steps. The first temperature selection at 37° C. in rich media with kanamycin produced single crossover recombinants, i.e., recombinants with integration of ts-sacB and concurrent deletion of the targeted region in LdhA. The second temperature selection at 33° C. on minimal media with sucrose produced double crossover recombinants, i.e., recombinants with subsequent loop out of all the pLCSac-LDH\hat{ } components, including the ts-sacB gene. Thus, transformants were selected for markerless and scarless genetic disruption of LdhA, producing the C. glutamicum recombinant strain LCG4004. Correct transformants were propagated on BHI+Kan.

### Example 2: Construction of Recombinant Corynebacterium glutamicum Strain LCG4021 with Eliminated Expression of Succinate Dehydrogenase

Example 2 describes the construction of succinate dehydrogenase (SDHCAB) minus C. glutamicum, LCG4021, wherein expression of SDHCAB in C. glutamicum (abbv. CgSDHC, UniProt ID: A0A1Q3DMH0, SEQ ID NO: 2, abbv. CgSDHA, UniProt ID: A0A072Z4F3, SEQ ID NO: 10; abbv. CgSDHB, UniProt ID: A0A1Q3DME3, SEQ ID NO: 11) was eliminated via genetic disruption of the succinate dehydrogenase genes C, A and B. LCG4012 cells with eliminated CgSDHCAB expression were unable to accumulate high amounts of succinate byproduct, thus not diverting carbon flux from aspartic acid/β-alanine pathway to the TCA cycle. The culturing and analysis of LCG4021 is described below in Examples 4 and 5.

CgSDHCAB was genetically disrupted using the ts-sacB markerless deletion methodology described above in Example 1 and disclosed in detail by Okibe et al in Journal of Microbiological Methods 85 (2011) 155-163. Briefly, a plasmid pLCSac-SDH\hat{ } was constructed to comprise the ts-sacB gene flanked by an upstream transcriptional promoter and a downstream transcriptional terminator. pLCSac-SDH\hat{ } also comprised unique upstream (SEQ ID NO: 5) and downstream (SEQ ID NO: 6) homologous regions to C. glutamicum SdhCAB for homologous recombination at the C. glutamicum SdhCAB locus. pLCSac-SDH\hat{ } further comprised a kanamycin resistant gene. Transformation of C. glutamicum recombinant strain LCG4001 with pLCSac-SDH\hat{ } was carried out according to the two-step process disclosed by Okibe et al, which comprised two temperature selection steps. The first temperature selection at 37° C. in rich media with kanamycin produced single crossover recombinants, i.e., recombinants with integration of ts-sacB gene and concurrent deletion of the targeted region in the SdhCAB locus. The second temperature selection at 33° C. on minimal media with sucrose produced double crossover recombinants, i.e., recombinants with subsequent loop out of all pLCSac-SDH\hat{ } components, including the ts-sacB gene. Thus, transformants were selected for markerless and scarless genetic disruption of SdhCAB, producing the C. glutamicum recombinant strain LCG4021. Correct transformants were propagated on BHI+Kan.

### Example 3: Construction of Recombinant Corynebacterium glutamicum Strain LCG4020 with Eliminated Expression of Lactate Dehydrogenase and Succinate Dehydrogenase

Example 3 describes the construction of lactate dehydrogenase (LDHA) minus and succinate dehydrogenase (SDHCAB) minus C. glutamicum, LCG4020, wherein expression of LDHA in C. glutamicum (abbv. CgLDHA; SEQ ID NO: 1) was eliminated via genetic disruption of the LdhA gene, and expression of SDHCAB in C. glutamicum (abbv. CgSDHC, UniProt ID: A0A1Q3DMH0, SEQ ID NO: 2, abbv. CgSDHA, UniProt ID: A0A072Z4F3, SEQ ID NO: 10; abbv. CgSDHB, UniProt ID: A0A1Q3DME3, SEQ ID NO: 11) was eliminated via genetic disruption of the succinate dehydrogenase genes C, A and B. LCG4020 cells were unable to accumulate high amounts of lactate and succinate byproducts, thus not diverting carbon flux from aspartic acid/β-alanine production. The culturing and analysis of LCG4020 is described below in Examples 4 and 5.

CgLDHA was genetically disrupted using the ts-sacB markerless deletion methodology described above in Example 1. CgSDHCAB was genetically disrupted using the ts-sacB markerless deletion methodology described above in Example 2. Transformants were selected for markerless and scarless genetic disruption of LdhA and SdhCAB, producing the C. glutamicum recombinant strain LCG4020. Correct transformants were propagated on BHI+Kan.

Strain LCG4020 described in this example is the background strain for C. glutamicum strains LCG4054, LCG4025, LCG4058, LCG4244, and LCG4062, both of which comprise an aspartic acid pathway of the present disclosure. The construction of strains LCG4054, LCG4025, LCG4058, LCG4244, and LCG4062 are described below in Example 6.

### Example 4: Culturing of Corynebacterium glutamicum Recombinant Strains LCG4004, LCG4021, and LCG4020, and Parent Strain LCG4002 Under Anaerobic Conditions

Example 4 describes the culturing of LCG4004 (LDHA minus), 4021 (SDHCAB minus) and 4020 (LDHA minus and SDHCAB minus) from Examples 1, 2, and 3, and the parent strain LCG4002 for the anaerobic production of lactate, succinate, and aspartic acid. These strains lacked heterologous nucleic acids encoding the aspartic acid pathway of the present disclosure. Each strain was first grown up from a single colony in a 250-mL baffled Erylenmyer flasks containing 50 mL of BHI+MOPS+glucose supplemented with 50 mM MOPS (pH 7.5), 2% glucose and 25 μg/mL kanamycin for 24 hours at 30° C. Cells grew for over 24 hours until OD₆₀₀ was ca. 10. Cultures were centrifuged at 4,000 x-g for 5 min; the media supernatant was discarded, and the cell pellet was resuspended with CGXII media to final OD₆₀₀ of ca. 15 g-dry cell weight (g-DCW). A 1 mL aliquot of CGXII cell suspension was transferred into multiple individual wells in a 96-deep well plate to make up technical replicates. The plate was covered with a breathable film and sealed in the commercially available BD Biosciences GasPak™ EZ container system to maintain an anaerobic environment. Briefly, an anaerobe sachet that acts as a catalyst to remove O₂ was incubated with the 96-deep well plate in the GasPak™ EZ container system. The BD Biosciences GasPak™ EZ container was incubated in a tabletop shaker with 330 rpm shaking at room temperature. Production runs were carried out for 90 hours to 150 hours and samples were analyzed periodically throughout production by collecting small samples from individual wells. In some cases, entire wells of cells were collected for analysis. Samples from wells were centrifuged or spin-filtered to separate cells from fermentation broth before the fermentation broth was analyzed by HPLC for the presence of lactate, succinate, and aspartic acid.

### Example 5: HPLC Analysis of Fermentation Broth of Corynebacterium glutamicum Recombinant Strains LCG4004, LCG4021, and LCG4020, and Parent Strain LCG4002 for the Presence of Lactate, Succinate, and Aspartic Acid

Example 5 describes HPLC analysis of recombinant C. glutamicum strains LCG4004 (LDHA minus), LCG4021 (SDHCAB minus), and LCG4020 (LDHA minus and SDHCAB minus) (constructed in Examples 1, 2, and 3, and cultured under anaerobic fermentation conditions in Example 4), and parent strain LCG4002 (also cultured as described in Example 4) for lactate, succinate, and aspartic acid production. All strains did not comprise either aspartic acid pathway of the present disclosure.

For HPLC analysis, each saved sample of fermentation broth from Example 4 was treated with o-phthalaldehyde (OPA) for derivatization, as recommended by Agilent, for use with an automated pre-column derivatization protocol that was integrated with HPLC analysis using the Agilent Zorbax Eclipse-AAA column (4.6 mm×75 mm, 3.5 micron). UV 338 nm measurements were acquired for 15 minutes.

For detection of sugars and organic acid by HPLC, the filtered samples were directly analyzed by HPLC, typically within 48 hours of harvest. Frozen samples were thawed analyzed by HPLC using a Bio-Rad Aminex 87H column (300×7.8 mm) and a Bio-Rad Fermentation Monitoring column (#1250115; 150×7.8 mm) installed in series, with an isocratic elution rate of 0.7 ml/min with water and 5 mM with sulfuric acid. Refractive index and UV 210 nm measurements were acquired for 20 minutes.

LCG4002 (parent C. glutamicum strain) produced 0.01 g/1-0.04 g/l of aspartic acid, indicative of basal level of aspartic acid production in C. glutamicum strains of the present disclosure. This demonstrates that all C. glutamicum strains lacking the aspartic acid pathway of the present disclosure are incapable of producing significant amounts of aspartic acid. Incorporation of heterologous nucleic acids that encode the aspartic acid pathway were later shown to enable increased aspartic acid production (Example 6). LCG4002 also produced 8 g/l-20 g/l of lactate and 3 g/l-10 g/l of succinate, indicating carbon flux diversion to the formation of byproducts lactate and succinate.

LCG4004 (LDHA minus C. glutamicum) produced only 0.4 g/l-3 g/l of lactate and 1 g/l-5 g/l of succinate. This result demonstrated that eliminated expression of CgLDHA significantly decreased the formation of lactate byproduct formation. LCG4004 did not produce detectable amounts of aspartic acid.

LCG4021 (SDHCAB minus C. glutamicum) produced 5 g/l-12 g/l of lactate and 0.1 g/1-4 g/l of succinate. This result demonstrated that eliminated expression of CgSDHCAB significantly decreased the formation of succinate byproduct formation. LCG4021 produced 0.01 g/1-0.1 g/l aspartic acid and a 0.5% yield (g-aspartic acid/g-glucose). This indicates that carbon flux from succinate byproduct formation pathways can be diverted to increase basal level production of aspartic acid.

While LCG4020 (LDHA minus and SDHCAB minus C. glutamicum) produced 0.1 g/1-0.5 g/l of lactate and 0.1 g/1-0.5 g/l of succinate. This result demonstrated that eliminated expression of CgLDHA and CgSDHCAB decreased the formation of lactate and succinate byproduct formation. LCG4020 also produced 0.1 g/1-0.3 g/l of aspartic acid and a 7% yield (g-aspartic acid/g-glucose). This indicates that it is possible to divert carbon flux from lactate and succinate byproduct formation towards increased basal level aspartic acid production.

### Example 6: Construction of Recombinant Corynebacterium glutamicum Strains LCG4054, LCG4025, LCG4058, LCG4244, and LCG4062 that Each Comprised an Aspartic Acid Pathway of the Present Disclosure

Example 6 describes the construction of recombinant C. glutamicum strains LCG4054, LCG4025, LCG4058, LCG4244, and LCG4062, wherein each strain comprised heterologous nucleic acids encoding enzymes of the aspartic acid pathway capable of carrying out the activities of phosphoenolpyruvate carboxykinase and aspartate transaminase or aspartate dehydrogenase (Table 1 and FIG. 1).

LCG4054 comprised the C. glutamicum phosphoenylpyruvate carboxykinase PckA UniProt ID: Q6F5A5 (abbv. CgPCKA, SEQ ID NO: 17) and the Variovorax sp. HW608 aspartate dehydrogenase UniProt ID: A0A1C6Q9L7 (abbv. AspDH #16, SEQ ID NO: 23). The heterologous nucleic acids encoding CgPCKA were amplified from C. glutamicum genomic DNA. The heterologous nucleic acids encoding AspDH #16 were codon-optimized for C. glutamicum and were synthesized and provided by Twist Bioscience.

Prior to LCG4054 strain construction, CgPCKA and AspDH #16 were cloned in tandem into plasmid pLCG1013 according to the SLIC method as described in detail by Li and Elledge in Methods Mol Biol (2012) 51-9, which is a method commonly practiced by practitioners of ordinary skill in the art. Plasmid pLCG1013 also comprised an upstream EF-Tu transcriptional promoter or an upstream Tac promoter, a downstream transcriptional terminator, and a kanamycin resistant gene. The LCG4020 strain in Example 3, which comprised LDHA minus and SDHCAB minus phenotype, was the background strain for LCG4054. LCG4020 was transformed with plasmid pLCG1013. Transformations were carried out in a single step. Transformants were propagated on BHI+Kan.

LCG4025 comprised the CgPCKA and the C. glutamicum aspartate transaminase UniProt ID: Q8NTR2 (abbv. CgASPB; SEQ ID NO: 25). The heterologous nucleic acids encoding CgPCKA were amplified from C. glutamicum genomic DNA. The heterologous nucleic acids encoding CgASPB were codon-optimized for C. glutamicum and were synthesized and provided by Twist Bioscience.

Prior to LCG4025 strain construction, CgPCKA and CgASPB were cloned in tandem into plasmid pLCD1002 according to the SLIC method. Plasmid pLCD1002 also comprised an upstream EF-Tu transcriptional promoter or an upstream Tac promoter, a downstream transcriptional terminator, and a kanamycin resistant gene. The LCG4020 strain in Example 3, which comprised LDHA minus and SDHCAB minus phenotype, was the background strain for LCG4025. LCG4020 was transformed with plasmid pLCG1002. Transformations were carried out in a single step. Transformants were propagated on BHI+Kan.

LCG4058 comprised the EcPCKA UniProt ID: P22259 and the CgAspB UniProt ID: Q8NTR2. The heterologous nucleic acids encoding EcPCKA was amplified from E. coli genomic DNA. The heterologous nucleic acids encoding CgAspB was amplified from C. glutamicum genomic DNA.

Prior to LCG4058 strain construction, EcPCKA and CgAspB were cloned in tandem into plasmid pCOMPASS-0031 according to the SLIC method. Plasmid pCOMPASS-0031 also comprised an upstream transcriptional promoter, a downstream transcriptional terminator, and a kanamycin resistant gene. The LCG4020 strain in Example 3, which comprised LDHA minus and SDHCAB minus phenotype, was the background strain for LCG4058.

LCG4020 was transformed with plasmid pCOMPASS-0031. Transformations were carried out in a single step. Transformants were propagated on BHI+Kan.

LCG4244 comprised the CgPCKA UniProt ID: Q6F5A5 and the AspDH #16 UniProt ID: A0A1C6Q9L7. The heterologous nucleic acids encoding EcPCKA was amplified from E. coli genomic DNA. The heterologous nucleic acids encoding CgAspB was amplified from C. glutamicum genomic DNA.

Prior to LCG4244 strain construction, CgPCKA and AspDH #16 were cloned in tandem into plasmid pCOMPASS-0131-2 according to the SLIC method. LCG4244 further comprised the Clostridium acetobutylium NADP⁺-utilizing GAPDH, i.e., Uniprot ID Q97D25 and abbv. CaGapC, which was cloned into plasmid pCOMPASS-0131-2. Plasmid pCOMPASS-0131-2 also comprised an upstream transcriptional promoter, a downstream transcriptional terminator, and a kanamycin resistant gene. Plasmid pCOMPASS-0131-2 was purified and provided as exogenous nucleic acids to the background strain LCG4248, which is LDHA minus, SDHCAB minus, ACKA minus, and PTA minus C. glutamicum, and was derived from LCG4020.

Prior to transformation with pCOMPASS-0131-2 to create LCG4244, LCG4020 (a LDHA minus and SDHCAB minus C. glutamicum) was constructed as described in Example 3. Using LCG4020 as a background strain, LCG4248 (a LDHA minus, SDHCAB minus, ACKA minus, and PTA minus C. glutamicum) was subsequently constructed using CRISPR methodology described by Cho et al in Metabolic Engineering 42 (2017) 157-67 and Wang et al in Microbial Cell Factories (2018) 17:63. Briefly, 2 plasmids were constructed: (1) pLC-Target was constructed to comprise gRNA for Cas9-ribonucleoprotein complex, spectinomycin selectable marker, 500-750 bp homology arm upstream and downstream of AckA-Pta; and (2) pLC1-Cas9-pTRC-RecE588T was constructed to comprise Cas9-ribonucleoprotein complex, RecE588-truncated exonuclease, RecT, pTrc inducible promoter driving RecE588T complex, and kanamycin selectable marker. All genes on both plasmids were codon-optimized for C. glutamicum. C. glutamicum was transformed via electroporation, first with the pLC1-Cas9-pTrc-RecE588T plasmid, then with the pLC-Target plasmid. Correct C. glutamicum transformants with desired AckA-Pta genetic disruptions were selected via propagation on BHI+Kan+Spec medium. Transformants were then cured of the pLC1-Cas9-pTRC-RecE588T plasmid, which was a temperature-sensitive plasmid that enabled practitioners to terminate the iterative knockout process and obtain plasmid-free strains (Cho et al, Metabolic Engineering 42 (2017) 157-67). Thus, transformants were grown at 37° C., rendered kanamycin-sensitive, and designated LCG4248. LCG4248 was then transformed with pCOMPASS-0131-2 and propagated on BHI-Kan to select for transformants which were designated LCG4244. This example describes construction of recombinant cells of the present disclosure LCG4248 which encode enzymes of the aspartic acid biosynthetic pathway of the present disclosure.

LCG4062 comprised the CgPCKA UniProt ID: Q6F5A5 and the Cupriavidus necator aspartate dehydrogenase UniProt ID: Q46VA0 (abbv. AspDH #13, SEQ ID NO: 9). The heterologous nucleic acids encoding CgPCKA were amplified from C. glutamicum genomic DNA. The heterologous nucleic acids encoding AspDH #13 were codon-optimized for C. glutamicum and were synthesized and provided by Twist Bioscience.

Prior to LCG4062 strain construction, CgPCKA and AspDH #13 were cloned in tandem into plasmid pCOMPASS-0034 according to the SLIC method. Plasmid pCOMPASS-0034 also comprised an upstream transcriptional promoter, a downstream transcriptional terminator, and a kanamycin resistant gene. The LCG4020 strain in Example 3, which comprised LDHA minus and SDHCAB minus phenotype, was the background strain for LCG4062. LCG4020 was transformed with plasmid pCOMPASS-0034. Transformations were carried out in a single step. Transformants were propagated on BHI+Kan.

This example describes construction of recombinant cells of the present disclosure LCG4054, LCG4025, LCG4058, LCG4244, and LCG4062 which encode enzymes of the aspartic acid biosynthetic pathway of the present disclosure.

### Example 7: Culturing and HPLC Analysis of Recombinant Corynebacterium glutamicum Strains LCG4054, LCG4025, and LCG4062 for Production of Aspartic Acid

Recombinant C. glutamicum strains LCG4054, LCG4025, and LCG4062 were cultured and analyzed by HPLC as described above in Examples 4 and 5 for the anaerobic production of aspartic acid. LCG4054, LCG4025, and LCG4062 each produced 5-13 g/l of aspartic acid and a 25-80% yield (g-aspartic acid/g-glucose). This example demonstrates, in accordance with the present disclosure, the expression of heterologous nucleic acids encoding an aspartic acid pathway in recombinant C. glutamicum that produced increased amounts of aspartic acid relative to the parental, control strains. The C. glutamicum background strain LCG4020 (described in Examples 4 and 5) lacked said heterologous aspartic acid pathway but is otherwise genetically identical; LCG4020 only produced 0.1 g/1-0.3 g/l of aspartic acid and 7% yield (g-aspartic acid/g-glucose).

### Example 8: Culturing and HPLC Analysis of Recombinant Corynebacterium glutamicum Strains LCG4054, LCG4025, and LCG4062 for Byproducts Lactate and Succinate

Recombinant C. glutamicum strains LCG4054, LCG4025, and LCG4062 were cultured and analyzed by HPLC as described above in Examples 4 and 5 for the byproducts lactate and succinate.

LCG4054 produced less than 0.5 g/l of lactate and less than 0.5 g/l of succinate. LCG4025 produced less than 0.5 g/l of lactate and less than 0.5 g/l of succinate. LCG4062 produced less than 1 g/l of lactate and less than 1.5 g/l of succinate.

This Example, taken together with Examples 4, 5, and 7, demonstrates minimal amounts of lactate and succinate were produced in aspartic acid recombinant cells that comprised LDHA minus and SDHCAB minus phenotypes.

### Example 9: Isolation of Aspartic Acid from 2-Liter Fermentation Broth

### Example 9a

Example 9a describes the isolation of aspartic acid from fermentation broth. LCG4058 was the strain used in this Example. LCG4058 comprised LDHA minus and SDHCAB minus phenotype and the corresponding genetic modifications were engineered as described in Example 3. LCG4058 also comprised the aspartic acid pathway, i.e., the CgAspB (UniProt ID: Q8NTR2) and EcPckA (UniProt ID: P22259), and the corresponding genetic modifications were engineered as described in Example 6.

LCG4058 was grown in 4-liters of BHI+MOPS+glucose medium supplemented with 25 μg/mL kanamycin. Cells were grown in a 5-liter bioreactor at 30° C. for about 45 hours to a cell density of about 4 g-DCW/1. Cells were pelleted by centrifugation at 4,000 x-g for 10 minutes and resuspended at about 7 g-DCW/1 in CGXII medium for a total of 2 liters. The resuspended cells were placed in a sealed fermentation bottle to provide an anerobic environment and the culture was incubated at 30° C. with sufficient shaking to prevent cells from settling. NaHCO₃ was used as the base during fermentation to maintain a fermentation pH of about 7. Aspartic acid titer at the end of a 300-hour fermentation was 25.9 g/l.

Cells were removed from the fermentation broth by centrifugation at 4,000 x-g for 10 minutes and the fermentation broth was concentrated to produce about 350 ml of clarified fermentation broth with an aspartic acid titer of 92 g/l (i.e., 699 mM). The total amount of aspartic acid in the clarified fermentation broth was calculated to be 32.6 g.

The clarified fermentation broth was concentrated by evaporation to increase the aspartic acid concentration. The concentrated solution of aspartic acid was then acidified by the addition of sulfuric acid to pH 2.5 to pH 3, which led to the crystallization of aspartic acid. Aspartic acid crystals were isolated by filtration with an 8-μm paper filter and the wet aspartic acid crystals were dried overnight in an oven at about 40° C. to about 50° C. Example 10a describes the characterization of isolated aspartic acid obtained from Example 9a.

### Example 9b

Example 9b describes the isolation of aspartic acid from fermentation broth. LCG4244 was the strain used in this Example. LCG4244 comprised LDHA minus, SDHCAB minus, ACKA minus, and PTA minus phenotype and the corresponding genetic modifications were engineered as described in Examples 3 and 6. LCG4244 also comprised the aspartic acid pathway, i.e., the CgPCKA UniProt ID: Q6F5A5 and the AspDH #16 UniProt ID: A0A1C6Q9L7, and the corresponding genetic modifications were engineered as described in Example 6.

LCG4244 was grown in 6-liters of BHI+MOPS+glucose medium supplemented with 25 μg/mL kanamycin. Cells were grown in 4-1.5-liter cultures (for a total of 6 liters) at 30° C. for about 24-28 hours to a cell density of OD₆₀₀ of about 5, which is about 1-2 g-DCW/1. Cells were pelleted by centrifugation at 4,000 x-g for 10 minutes and resuspended at about 10-15 g-DCW/1 in CGXII medium for a total of 1,800 ml. The resuspended cells were divided into 6-300 ml cultures in fermentation bottles. The fermentation bottles were sealed to provide an anerobic environment and the cultures were incubated at 37° C. with sufficient shaking to prevent cells from settling. Ammonium bicarbonate was used as the base during fermentation to maintain a fermentation pH of about 7. Aspartic acid titer at the end of a 72-hour fermentation was 54.3 g/l.

Cells were removed from the fermentation broth by centrifugation at 4,000 x-g for 10 minutes and by microfiltration with a 0.2-μm filter to produce about 400 ml of clarified fermentation broth with an aspartic acid titer of 54.3 g/l (i.e., 398 mM). The total amount of aspartic acid in the clarified fermentation broth was calculated to be 21.7 g. The 400 ml of clarified fermentation broth was filtered with activated carbon to remove colored impurities, and evaporated to increase the concentration of aspartic acid. The concentrated solution of aspartic acid, or the concentrated clarified fermentation broth was then acidified by the addition of hydrochloric acid to pH 2.5 to pH 3, which led to the precipitation of aspartic acid. Precipitated aspartic acid was redissolved at 90° C. and the solution of aspartic acid was slowly cooled down to 5° C. for crystallization. The mother liquor from this crystallization step was taken through another round of crystallization which comprised the steps of concentrating, heating, and slow cooling to increase the amount of total aspartic acid crystals recovered. Aspartic acid crystals from both rounds of crystallization were isolated by filtration with an 8-μm paper filter and the wet aspartic acid crystals were dried overnight in an oven at about 40° C. to about 50° C. Example 10b describes the characterization of isolated aspartic acid obtained from Example 9b.

### Example 10a: Characterization of Isolated Aspartic Acid

Aspartic acid from Example 9a was prepared for HPLC analysis using an OPA derivatization method. The aspartic acid solution was determined to have 93.3% purity. 30.74 g+/−2.975 g of aspartic acid was recovered, which converts to a recovery yield from fermentation broth of 94.24+/−11.79%.

### Example 10b: Characterization of Isolated Aspartic Acid

Aspartic acid produced as provided herein was prepared for GC-FID analysis using a TMS derivatization method. The aspartic acid solution was determined to have 99.7% purity (peak area %). About 17.2 g of aspartic acid was recovered, which converts to a recovery yield from fermentation broth of about 79%.

### Example 11: Construction of Recombinant Corynebacterium glutamicum Strains LCG4133, LCG4166, LCG4136, and LCG4137 that Each Comprised an Aspartic Acid Pathway and Heterologous Nucleic Acids Encoding a NADP⁺- or NAD⁺-Utilizing GAPDH of the Present Disclosure

Example 11 describes the construction of recombinant C. glutamicum strains LCG4133, LCG4166, LCG4136 and LCG4137, wherein each strain comprised heterologous nucleic acids encoding enzymes of the aspartic acid pathway capable of carrying out the activities of phosphoenolpyruvate carboxykinase and aspartate dehydrogenase (Table 1 and FIG. 1). These 4 strains further comprised heterologous nucleic acids encoding either a NADP⁺-utilizing GAPDH or a NAD⁺-utilizing GAPDH.

LCG4133 comprised the C. glutamicum phosphoenylpyruvate carboxykinase PckA UniProt ID: Q6F5A5 (abbv. CgPCKA, SEQ ID NO: 17), the Variovorax sp. HW608 aspartate dehydrogenase UniProt ID: A0A1C6Q9L7 (abbv. AspDH #16), and the Clostridium acetobutylium NADP⁺-utilizing GAPDH, i.e., Uniprot ID Q97D25 (abbv. CaGapC). CgPCKA, AspDH #16, and CaGapC were cloned into plasmid pCOMPASS-0131 according to the methods described in Example 6. Prior to transformation with pCOMPASS-0131 to create LCG4133, LCG4020 (a LDHA minus and SDHCAB minus C. glutamicum) was constructed as described in Example 3. Using LCG4020 as a background strain, LCG4133 was constructed as described in Example 6. This example describes construction of recombinant cells of the present disclosure LCG4133 which encode enzymes of the aspartic acid biosynthetic pathway of the present disclosure and CaGapC.

LCG4166 comprised the C. glutamicum phosphoenylpyruvate carboxykinase PckA UniProt ID: Q6F5A5 (abbv. CgPCKA, SEQ ID NO: 17), the Variovorax sp. HW608 aspartate dehydrogenase UniProt ID: A0A1C6Q9L7 (abbv. AspDH #16), and the Methanococcus maripaludis NADP⁺-utilizing GAPDH, i.e., Uniprot ID Q97D25 (abbv. MmGapC). CgPCKA, AspDH #16, and MmGapC were cloned into plasmid pCOMPASS-0140 as described in Example 6. Prior to transformation with pCOMPASS-0140 to create LCG4166, LCG4020 (a LDHA minus and SDHCAB minus C. glutamicum) was constructed as described in Example 3. Using LCG4020 as a background strain, LCG4166 was constructed as described in Example 6. This example describes construction of recombinant cells of the present disclosure LCG4166 which encode enzymes of the aspartic acid biosynthetic pathway of the present disclosure and MmGapC.

LCG4136 comprised the C. glutamicum phosphoenylpyruvate carboxykinase PckA UniProt ID: Q6F5A5 (abbv. CgPCKA, SEQ ID NO: 17), the Variovorax sp. HW608 aspartate dehydrogenase UniProt ID: A0A1C6Q9L7 (abbv. AspDH #16), and the Corynebacterium glutamicum NAD⁺-utilizing GAPDH, i.e., Uniprot ID A0A0U4IQV8 (abbv. CgGapX). CgPCKA, AspDH #16, and CgGapX were cloned into plasmid pCOMPASS-0136 as described in Example 6. Prior to transformation with pCOMPASS-0136 to create LCG4136, LCG4020 (a LDHA minus and SDHCAB minus C. glutamicum) was constructed as described in Example 3. Using LCG4020 as a background strain, LCG4136 was constructed as described in Example 6. This example describes construction of recombinant cells of the present disclosure LCG4136 which encode enzymes of the aspartic acid biosynthetic pathway of the present disclosure and CgGapX.

LCG4137 comprised the C. glutamicum phosphoenylpyruvate carboxykinase PckA UniProt ID: Q6F5A5 (abbv. CgPCKA, SEQ ID NO: 17), the Variovorax sp. HW608 aspartate dehydrogenase UniProt ID: A0A1C6Q9L7 (abbv. AspDH #16), and the Corynebacterium glutamicum NAD⁺-utilizing GAPDH, i.e., Uniprot ID P0A9B2 (abbv. EcGapA). CgPCKA, AspDH #16, and EcGapA were cloned into plasmid pCOMPASS-0137 as described in Example 6. Prior to transformation with pCOMPASS-0137 to create LCG4137, LCG4020 (a LDHA minus and SDHCAB minus C. glutamicum) was constructed as described in Example 3. Using LCG4020 as a background strain, LCG4137 was constructed as described in Example 6. This example describes construction of recombinant cells of the present disclosure LCG4137 which encode enzymes of the aspartic acid biosynthetic pathway of the present disclosure and EcGapA.

The culturing and analysis of LCG4133, LCG4166, LCG4136, and LCG4137 are described below in Example 12.

### Example 12: Culturing and HPLC Analysis of Recombinant Corynebacterium glutamicum Strains LCG4133, LCG4166, LCG4136, and LCG4137 for Production of Aspartic Acid

Recombinant C. glutamicum strains LCG4133, LCG4166, LCG4136 and LCG4137 were cultured and analyzed by HPLC as described above in Examples 4 and 5 for the anaerobic production of aspartic acid. Aspartic acid titers at 24 and 72 hours of fermentation are as follow: (1) LCG4133 (with overexpression of NADP⁺-utilizing GAPDH CaGapC) produced β-18 g/l and 25-30 g/l, respectively; (2) LCG4166 (with overexpression of NADP⁺-utilizing GAPDH MmGapC) produced 10-15 g/l and 25-30 g/l, respectively; (3) LCG4136 (with overexpression of NAD⁺-utilizing GAPDH CgGapX) produced 0-3 g/l and 5-8 g/l, respectively; and (4) LCG 4137 (with overexpression of NAD⁺-utilizing GAPDH EcGapA) produced 5-8 g/l and 15-20 g/l, respectively. This Example demonstrates that overexpression of the NADP⁺-utilizing GAPDHs, CaGapC and MmGapC, improved aspartic acid production while overexpression of the NAD⁺-utilizing GAPDHs, EcGapC and CgGapC, either did not improve or inhibited aspartic acid production. This Example demonstrates that a aspartic acid pathway of the present disclosure had a preference for NADP⁺ co-factor that was satisfied, resulting in improved aspartic acid production.

Various publications were referenced in this application. The disclosures of these publications in their entireties are hereby incorporated by reference in this application in order to more fully describe the state of the art to which this invention pertains.

It should be noted that there are alternative ways of implementing the embodiments disclosed herein. Accordingly, the present embodiments are to be considered as illustrative and not restrictive; various modifications can be made without departing from the spirit of the invention. Furthermore, the claims are not to be limited to the details given herein, and are entitled their full scope and equivalents thereof.

1. A recombinant host cell comprising:
(a) one or more heterologous nucleic acids encoding an aspartate-forming enzyme either selected from the group consisting of aspartate dehydrogenase and aspartate transaminase or selected from a sequence having at least 95% amino acid identity with SEQ ID NO: 23, AspDH #15, AspDH #17, AspDH #18, or AspDH #20; and
(b) one or more heterologous nucleic acids encoding an oxaloacetate-forming enzyme selected from the group consisting of pyruvate carboxylase, phosphoenolpyruvate carboxylase, and phosphoenolpyruvate carboxykinase.

2. The recombinant host cell of claim 1, further comprising one or more heterologous nucleic acids encoding an aspartate 1-decarboxylase.

3. The recombinant host cell of claim 1, wherein the recombinant host cell is capable of producing aspartate under anaerobic conditions.

4. (canceled)

5. The recombinant host cell of claim 1, wherein the recombinant host cell is a bacterial cell.

6. The recombinant host cell of claim 1, wherein the recombinant host cell is Escherichia coli, Corynebacterium glutamicum, or Pantoea ananatis.

7. The recombinant host cell of claim 1, wherein the aspartate dehydrogenase is selected from the group consisting SEQ ID NO: 9, SEQ ID NO: 19, SEQ ID NO: 20, SEQ ID NO: 21, SEQ ID NO: 22, SEQ ID NO: 23 and SEQ ID NO: 24.

8. The recombinant host cell of claim 1, wherein the aspartate dehydrogenase has at least 40% amino acid identity with SEQ ID NO: 33.

9. The recombinant host cell of claim 1, wherein the aspartate transaminase is selected from the group consisting SEQ ID NO: 25, SEQ ID NO: 26, SEQ ID NO: 27 and SEQ ID NO: 28.

10. The recombinant host cell of claim 1, wherein the aspartate transaminase has at least 40% amino acid identity with SEQ ID NO: 36.

11. The recombinant host cell of claim 1, wherein the pyruvate carboxylase is SEQ ID NO: 15.

12. The recombinant host cell of claim 1, wherein the phosphoenolpyruvate carboxykinase is selected from the group consisting SEQ ID NO: 16, SEQ ID NO: 17, and SEQ ID NO: 18.

13. The recombinant host cell of claim 1, wherein the phosphoenolpyruvate carboxylase is selected from the group consisting SEQ ID NO: 12, SEQ ID NO: 13, and SEQ ID NO: 14.

14. The recombinant host cell of claim 1, wherein the phosphoenolpyruvate carboxylase has at least 40% amino acid identity with SEQ ID NO: 35.

15. The recombinant host cell of claim 2, wherein the aspartate 1-decarboxylase is selected from the group consisting SEQ ID NO: 29, SEQ ID NO: 37, and SEQ ID NO: 38.

16. The recombinant host cell of claim 1, wherein the phosphoenolpyruvate carboxylase has at least 40% amino acid identity with SEQ ID NO: 39 or SEQ ID NO: 40.

17. The recombinant host cell of claim 1, further comprising one or more disruptions of one or more genes encoding a succinate dehydrogenase subunit.

18. The recombinant host cell of claim 17, wherein the succinate dehydrogenase subunit is selected from the group consisting SEQ ID NO: 2, SEQ ID NO: 10, and SEQ ID NO: 11.

19. The recombinant host cell of claim 17, wherein the succinate dehydrogenase subunit has at least 40% amino acid identity with SEQ ID NO: 2, SEQ ID NO: 10, or SEQ ID NO: 11

20-22. (canceled)

23. A method of producing aspartic acid or b-alanine comprising the step of culturing the recombinant host cell of claim 1 to produce aspartic acid or b-alanine.

24-30. (canceled)

31. A method for isolating aspartic acid or a salt thereof, comprising:
culturing the recombinant host cell of claim 1 in a fermentation broth to produce aspartic acid or a salt thereof;
separating the recombinant host cell from the fermentation broth to produce a clarified fermentation broth;
optionally, concentrating the clarified fermentation broth to provide a concentrated fermentation broth;
optionally contacting the concentrated fermentation broth with an ion exchange resin or activated carbon adsorbent;
acidifying the clarified fermentation broth or the concentrated fermentation broth to precipitate the aspartic acid or the salt thereof; and
isolating the precipitated aspartic acid or the salt thereof.

32. The method of claim 31, wherein the fermentation broth is maintained at a pH of about 6 to about pH 8.

33-43. (canceled)

44. The method of claim 31, wherein the isolated aspartic acid or the salt thereof has a purity of about 90% or more.


SEQUENCE LISTING (54 sequences)

SEQ ID NO: 1 - protein, 314 aa, Corynebacterium glutamicum
MKETVGNKIVLIGAGDVGVAYAYALINQGMADHLAIIDIDEKKLEGNVMDLNHGVVWADSRTRVTKGTYADCEDAAMVVICAGAAQKPGETRLQLVDKNVKIMKSIVGDVMDSGFDGIFLVASNPVDILTYAVWKFSGLEWNRVIGSGTVLDSARFRYMLGELYEVAPSSVHAYIIGEHGDTELPVLSSATIAGVSLSRMLDKDPELEGRLEKIFEDTRDAAYHIIDAKGSTSYGIGMGLARITRAILQNQDVAVPVSALLHGEYGEEDIYIGTPAVVNRRGIRRVVELEITDHEMERFKHSANTLREIQKQFF

SEQ ID NO: 2 - protein, 257 aa, Corynebacterium glutamicum
MTVRNPDREAIRHGKITTEALRERPAYPTWAMKLTMAITGLMFGGFVLVHMIGNLKIFMPDYAADSAHPGEAQVDVYGEFLREIGSPILPHGSVLWILRIILLVALVLHIYCAFALTGRSHQSRGKFRRTNLVGGFNSFATRSMLVTGIVLLAFIIFHILDLTMGVAPAAPTSFEHGEVYANMVASFSRWPVAIWYIIANLVLFVHLSHGIWLAVSDLGITGRRWRAILLAVAYIVPALVLIGNITIPFAIAVGWIA

SEQ ID NO: 3 - DNA, 750 bp, Corynebacterium glutamicum
agccgtaacccaccacggtttcggcaacaatgacggcgagagagcccaccacattgcgatttccgctccgataaagccagcgcccatatttgcagggaggattcgcctgcggtttggcgacattcggatccccggaactagctctgcaatgacctgcgcgccgagggaggcgaggtgggtggcaggttttagtgcgggtttaagcgttgccaggcgagtggtgagcagagacgctagtctggggagcgaaaccatattgagtcatcttggcagagcatgcacaattctgcagggcataggttggttttgctcgatttacaatgtgattttttcaacaaaaataacacttggtctgaccacattttcggacataatcgggcataattaaaggtgtaacaaaggaatccgggcacaagctcttgctgattttctgagctgctttgtgggttgtccggttagggaaatcaggaagtgggatcgaaaatgaaagaaaccgtcggtaacaagattgtcctcattggcgcaggagatgttggagttgcatacgcatacgcactgatcaaccagggcatggcagatcaccttgcgatcatcgacatcgatgaaaagaaactcgaaggcaacgtcatggacttaaaccatggtgttgtgtgggccgattcccgcacccgcgtcaccaagggcacctacgctgactgcgaagacgcagccatggttgtcatttgtgccggcgcagcccaaaagccaggc

SEQ ID NO: 4 - DNA, 750 bp, Corynebacterium glutamicum
atcacattatcgacgccaagggctccacttcctacggcatcggcatgggtcttgctcgcatcacccgcgcaatcctgcagaaccaagacgttgcagtcccagtctctgcactgctccacggtgaatacggtgaggaagacatctacatcggcaccccagctgtggtgaaccgccgaggcatccgccgcgttgtcgaactagaaatcaccgaccacgagatggaacgcttcaagcattccgcaaataccctgcgcgaaattcagaagcagttcttctaaatctttggcgcctagttggcgacgcaagtgtttcattggaacacttgcgctgccaactttttggtttacgggcacaatgaaactgttggatggaatttagagtgtttgtagcttaaggagctcaaatgaatgagtttgaccaggacattctccaggagatcaagactgaactcgacgagttaattctagaacttgatgaggtgacacaaactcacagcgaggccatcgggcaggtctccccaacccattacgttggtgcccgcaacctcatgcattacgcgcatcttcgcaccaaagacctccgtggcctgcagcaacgcctctcctctgtgggagctacccgcttgactaccaccgaaccagcagtgcaggcccgcctcaaggccgcccgcaatgttatcggagctttcgcaggtgaaggcccactttatccaccctcagatgtcgtcgatgccttcgaagatgccgatga

SEQ ID NO: 5 - DNA, 885 bp, Corynebacterium glutamicum
gtgagcgggcgtacgtcgtgctcaatctgatttacataacttgcagataagccaagggttgctgctaaggatgcctggctcaggtctctttcgcggcgcagttggcgcagcctggaccccacatatgtctttcccatggcgcaactatagcgtgatcaccatcaccttaaccactcttggcactgaggtgttgcaaacttgttgattttcgcttttcgacgcagcccgccgccaccaggtgcccggcgtggtcgggccacatccgccccgggaactttttaggcacctacggtgcaactgttgggataattgtgtcacctgcgcaaagttgctccctggatcggaaggttgggctgtctaaactttttggttgataccaaacggggttagaaactgttcggatcggtatcctgtgaggaagctcaccttggttttagaatgttgaaaaggcctcacgtttccgcaggtagagcacactcaattaaatgagcgtcaaacgacaataaagtaaggctatcctaataagtggggttttatgtctctaaacagccagttgggggtcatgggggagcgccccgtgactggttaatgccccgatctgggacgtacagtaacaacgacactggaggtgccatgactgttagaaatcccgaccgtgaggcaatccgtcacggaaaaattacgacggaggcgctgcgtgagcgtcccgcatacccgacctgggcaatgaagctgaccatggccatcactggcctaatgtttggtggcttcgttcttgttcacatgatcggaaacctgaaaatcttcatgccggactacgcagccgattctgcgcatccgggtgaagcacaagtagatgtctacggcgagttcctgcgtgagatc

SEQ ID NO: 6 - DNA, 885 bp, Corynebacterium glutamicum
cacgtaagatggttgatgaaatggaaaccaacttcggacactgctccctctacggcgagtgcgcagatgtctgccccgcaggcatcccactgaccgctgtggcagctgtcaccaaggaacgtgcgcgtgcagctttccgaggcaaagacgactagtctttaatccaagtaagtaccggttcagacagttaaaccagaaagacgagtgaacaccatgtcctccgcgaaaaagaaacccgcaccggagcgtatgcactacatcaagggctatgtacctgtggcgtatagctctccacactcatccctcgagcgcagcgcaacctggttgggcatgggattcctcctcactgctctggcaggcgttggcgcagtcctcttcgcagtcggcgcaaacagcgttggccagcagcaggaacactgggtcctctacagcatcatcggtgttgtattcgccgttgtctgcacagttttgggcaccgtcctgatcatcaagggccgagcaccttacaaccgttacgtcaaggaaaccggccgtacgcagtagtttctgtatgcaggttctttgactagcacctccaacaacaccctttcttccatttatgtgggggagagggtgttgttttgtttttgcttggagttccctggagccttcatcgacagcccccaagccctcgaaaacgccgaaagcaacccactgtggagccaactccccgcagtcaaaaacggtcaactctgtaccacggaaaacctcaccccatggatcctcaccggaccagcagcagctgagattgtgacctctgacctcgaggcatgtttcgctgcttaggagacatggaagattggtggaaattgggtcgtgcagaaggatgcttggagatataggata

SEQ ID NO: 7 - protein, 1517 aa, Kluyveromyces marxianus
MSNSSSSEGKTNEDGRNSVHSSDSFAQSVASFHLDDNESQNVTAQLSQQITNVLSNSNGAERIESLARVISTKTKKQMESFEVNQLDFDLKALLNYLRSSQLEQGIEPGDSGIAFHDLTAVGIDASAAFGPSVEEMVRSWIHFPVRLWKKICRQKSETPLRNIIQHCTGVVESGEMLFVVGRPGAGCSTLLKCLSGETGELVEVTGDISYDGLSQEEMMQKFKGYVIYCPELDFHFPKITVKETIDFALKCKTPRSRIDHLTRAQYVDNMRDLWCTVFGLTHTYATNVGNDVVRGVSGGERKRVSLVEALAMNASIYSWDNATRGLDASTALEFAQAIRTATNMMNNSAIVAIYQAGENIYQLFDKTTVLYNGKQVYFGPADEAVGYFERMGYIKPNRMTSAEFLTSATVDFENRTLEVREGYEEKIPKSSTEMEAYWHNSPEYAKATELFNEYCQSHPEEETRQRLETAKKQRLQKGQREKSQFVVTFWAQVWYCMIRGFQRVKGDSTYTKVYLSSFLTKGLIVGSMFHKIDPKSQSTTEGAYSRGGLLFYVLLFAALTSLAEISNSFQNRAIIVKQKTYSMYHTSAESLQEIFTEIPTKFVAILTLSLVSYWIPVLKYDAGSFFQYLLYLFTTQQCTSFIFKLVATLTKDGGTAHAIGGLWVLMLTVYAGFVLPIGNMHHWIRWFHYLNPLTYAYESLMSTEFHGRKMLCSRLLPSGPGYENVSIAHKICDAAGAVAGQLYVSGDAYVLKKYHFRYKHAWRDWGINIVWTFGYIVMNVVMSEYLKPLEGGGDLLLYKRGHMPELGSESVDSKVASREEMMESLNGPGVDLEKVIASKDVFTWNHLNYTIPYDGATRQLLSDVFGYVKPGKMTALMGESGAGKTTLLNVLAQRINVGVITGDMLVNAKPLPPSFNRSCGYVAQADNHMGELSVRESLRFAAELRQPKSVPLQEKYDYVEKIISLLGMEKYAEAIIGKTGRGLNVEQRKKLSIGVELVAKPSLLLFLDEPTSGLDSQSAWSIVQFMRALADSGQSILCTIHQPSATLFEQFDRLLLLKKGGKMVYFGDIGENSSTLLNYFERQSGVKCGKSENPAEYMLNCIGAGATASADADWHDLWLQSPECAAAREEVEELHRTLASRPVTDDKELAGRYAASYLTQMKCVFRRTNIQFWRSPVYIRAKFLECVLCALFVGLSYVGVDHSIAGASQSFSSIFMMLLIALAMVNQLHVFALDSRELYEVREAASNTFHWSVLLLNHTFVEIIWSTLCEFICWICYYWPAQYSGRASHAGYFFLIYVIMFPAYFVSYGCWVFYMSPDVPSASMINSNLFAGMLLFCGILQPKDKMPGFWKRFMYNVSPFTYVVQSLVTPLVQGKKVRCTKNEFAVVNPPEGQTCSQYFARFIKDNTGYLKNPNDTESCHYCPYSYQQEVVEQYNVRWVYRWRNFGFLWAYIGFNFFAMLACYWVLRVKNYSITSIFGVFKIGNWKKAIHHDSRHEKDHTIFQEKPGDAANVQKTKA

SEQ ID NO: 8 - protein, 328 aa, Corynebacterium glutamicum
MNSPQNVSTKKVTVTGAAGQISYSLLWRIANGEVFGTDTPVELKLLEIPQALGGAEGVAMELLDSAFPLLRNITITADANEAFDGANAAFLVGAKPRGKGEERADLLANNGKIFGPQGKAINDNAADDIRVLVVGNPANTNALIASAAAPDVPASRFNAMMRLDHNRAISQLATKLGRGSAEFNNIVVWGNHSATQFPDITYATVGGEKVTDLVDHDWYVEEFIPRVANRGAEIIEVRGKSSAASAASSAIDHMRDWVQGTEAWSSAAIPSTGAYGIPEGIFVGLPTVSRNGEWEIVEGLEISDFQRARIDANAQELQAEREAVRDLL

SEQ ID NO: 9 - protein, 268 aa, Cupriavidus necator
MSMLHVSMVGCGAIGRGVLELLKADPDVAFDVVIVPEGQMDEARSALSALAPNVRVATGLDGQRPDLLVECAGHQALEEHIVPALERGIPCMVVSVGALSEPGLVERLEAAARRGNTQVQLLSGAIGAIDALAAARVGGLDEVIYTGRKPARAWTGTPAAELFDLEALTEPTVIFEGTARDAARLYPKNANVAATVSLAGLGLDRTSVRLLADPNAVENVHHIEARGAFGGFELTMRGKPLAANPKTSALTVFSVVRALGNRAHAVSI

SEQ ID NO: 10 - protein, 673 aa, Corynebacterium glutamicum
MSTHSETTRPEFIHPVSVLPEVSAGTVLDAAEPAGVPTKDMWEYQKDHMNLVSPLNRRKFRVLVVGTGLSGGAAAAALGELGYDVKAFTYHDAPRRAHSIAAQGGVNSARGKKVDNDGAYRHVKDTVKGGDYRGRESDCWRLAVESVRVIDHMNAIGAPFAREYGGALATRSFGGVQVSRTYYTRGQTGQQLQLSTASALQRQIHLGSVEIFTHNEMVDVIVTERNGEKRCEGLIMRNLITGELTAHTGHAVILATGGYGNVYHMSTLAKNSNASAIMRAYEAGAYFASPSFIQFHPTGLPVNSTWQSKTILMSESLRNDGRIWSPKEPNDNRDPNTIPEDERDYFLERRYPAFGNLVPRDVASRAISQQINAGLGVGPLNNAAYLDFRDATERLGQDTIRERYSNLFTMYEEAIGEDPYSSPMRIAPTCHFTMGGLWTDFNEMTSLPGLFCAGEASWTYHGANRLGANSLLSASVDGWFTLPFTIPNYLGPLLGSERLSEDAPEAQAAIARAQARIDRLMGNRPEWVGDNVHGPEYYHRQLGDILYFSCGVSRNVEDLQDGINKIRALRDDFWKNMRITGSTDEMNQVLEYAARVADYIDLGELMCVDALDRDESCGAHFRDDHLSEDGEAERDDENWCFVSAWEPGENGTFVRHAEPLFFESVPLQTRNYK

SEQ ID NO: 11 - protein, 673 aa, Corynebacterium glutamicum; identical to SEQ ID NO: 10

SEQ ID NO: 12 - protein, 883 aa, Escherichia coli
MNEQYSALRSNVSMLGKVLGETIKDALGEHILERVETIRKLSKSSRAGNDANRQELLTTLQNLSNDELLPVARAFSQFLNLANTAEQYHSISPKGEAASNPEVIARTLRKLKNQPELSEDTIKKAVESLSLELVLTAHPTEITRRTLIHKMVEVNACLKQLDNKDIADYEHNQLMRRLRQLIAQSWHTDEIRKLRPSPVDEAKWGFAVVENSLWQGVPNYLRELNEQLEENLGYKLPVEFVPVRFTSWMGGDRDGNPNVTADITRHVLLLSRWKATDLFLKDIQVLVSELSMVEATPELLALVGEEGAAEPYRYLMKNLRSRLMATQAWLEARLKGEELPKPEGLLTQNEELWEPLYACYQSLQACGMGIIANGDLLDTLRRVKCFGVPLVRIDIRQESTRHTEALGELTRYLGIGDYESWSEADKQAFLIRELNSKRPLLPRNWQPSAETREVLDTCQVIAEAPQGSIAAYVISMAKTPSDVLAVHLLLKEAGIGFAMPVAPLFETLDDLNNANDVMTQLLNIDWYRGLIQGKQMVMIGYSDSAKDAGVMAASWAQYQAQDALIKTCEKAGIELTLFHGRGGSIGRGGAPAHAALLSQPPGSLKGGLRVTEQGEMIRFKYGLPEITVSSLSLYTGAILEANLLPPPEPKESWRRIMDELSVISCDVYRGYVRENKDFVPYFRSATPEQELGKLPLGSRPAKRRPTGGVESLRAIPWIFAWTQNRLMLPAWLGAGTALQKVVEDGKQSELEAMCRDWPFFSTRLGMLEMVFAKADLWLAEYYDQRLVDKALWPLGKELRNLQEEDIKVVLAIANDSHLMADLPWIAESIQLRNIYTDPLNVLQAELLHRSRQAEKEGQEPDPRVEQALMVTIAGIAAGMRNTG

SEQ ID NO: 13 - protein, 606 aa, Mycobacterium tuberculosis
MTSATIPGLDTAPTNHQGLLSWVEEVAELTQPDRVVFTDGSEEEFQRLCDQLVEAGTFIRLNPEKHKNSYLALSDPSDVARVESRTYICSAKEIDAGPTNNWMDPGEMRSIMKDLYRGCMRGRTMYVVPFCMGPLGAEDPKLGVEITDSEYVVVSMRTMTRMGKAALEKMGDDGFFVKALHSVGAPLEPGQKDVAWPCSETKYITHFPETREIWSYGSGYGGNALLGKKCYSLRIASAMAHDEGWLAEHMLILKLISPENKAYYFAAAFPSACGKTNLAMLQPTIPGWRAETLGDDIAWMRFGKDGRLYAVNPEFGFFGVAPGTNWKSNPNAMRTIAAGNTVFTNVALTDDGDVWWEGLEGDPQHLIDWKGNDWYFRETETNAAHPNSRYCTPMSQCPILAPEWDDPQGVPISGILFGGRRKTTVPLVTEARDWQHGVFIGATLGSEQTAAAEGKVGNVRRDPMAMLPFLGYNVGDYFQHWINLGKHADESKLPKVFFVNWFRRGDDGRFLWPGFGENSRVLKWIVDRIEHKAGGATTPIGTVPAVEDLDLDGLDVDAADVAAALAVDADEWRQELPLIEEWLQFVGEKLPTGVKDEFDALKERLG

SEQ ID NO: 14 - protein, 919 aa, Corynebacterium glutamicum
MTDFLRDDIRFLGQILGEVIAEQEGQEVYELVEQARLTSFDIAKGNAEMDSLVQVFDGITPAKATPIARAFSHFALLANLAEDLYDEELREQALDAGDTPPDSTLDATWLKLNEGNVGAEAVADVLRNAEVAPVLTAHPTETRRRTVFDAQKWITTHMRERHALQSAEPTARTQSKLDEIEKNIRRRITILWQTALIRVARPRIEDEIEVGLRYYKLSLLEEIPRINRDVAVELRERFGEGVPLKPVVKPGSWIGGDHDGNPYVTAETVEYSTHRAAETVLKYYARQLHSLEHELSLSDRMNKVTPQLLALADAGHNDVPSRVDEPYRRAVHGVRGRILATTAELIGEDAVEGVWFKVFTPYASPEEFLNDALTIDHSLRESKDVLIADDRLSVLISAIESFGFNLYALDLRQNSESYEDVLTELFERAQVTANYRELSEAEKLEVLLKELRSPRPLIPHGSDEYSEVTDRELGIFRTASEAVKKFGPRMVPHCIISMASSVTDVLEPMVLLKEFGLIAANGDNPRGTVDVIPLFETIEDLQAGAGILDELWKIDLYRNYLLQRDNVQEVMLGYSDSNKDGGYFSANWALYDAELQLVELCRSAGVKLRLFHGRGGTVGRGGGPSYDAILAQPRGAVQGSVRITEQGEIISAKYGNPETARRNLEALVSATLEASLLDVSELTDHQRAYDIMSEISELSLKKYASLVHEDQGFIDYFTQSTPLQEIGSLNIGSRPSSRKQTSSVEDLRAIPWVLSWSQSRVMLPGWFGVGTALEQWIGEGEQATQRIAELQTLNESWPFFTSVLDNMAQVMSKAELRLAKLYADLIPDTEVAERVYSVIREEYFLTKKMFCVITGSDDLLDDNPLLARSVQRRYPYLLPLNVIQVEMMRRYRKGDQSEQVSRNIQLTMNGLSTALRNSG

SEQ ID NO: 15 - protein, 1140 aa, Corynebacterium glutamicum
MSTHTSSTLPAFKKILVANRGEIAVRAFRAALETGAATVAIYPREDRGSFHRSFASEAVRIGTEGSPVKAYLDIDEIIGAAKKVKADAIYPGYGFLSENAQLARECAENGITFIGPTPEVLDLTGDKSRAVTAAKKAGLPVLAESTPSKNIDEIVKSAEGQTYPIFVKAVAGGGGRGMRFVASPDELRKLATEASREAEAAFGDGAVYVERAVINPQHIEVQILGDHTGEVVHLYERDCSLQRRHQKVVEIAPAQHLDPELRDRICADAVKFCRSIGYQGAGTVEFLVDEKGNHVFIEMNPRIQVEHTVTEEVTEVDLVKAQMRLAAGATLKELGLTQDKIKTHGAALQCRITTEDPNNGFRPDTGTITAYRSPGGAGVRLDGAAQLGGEITAHFDSMLVKMTCRGSDFETAVARAQRALAEFTVSGVATNIGFLRALLREEDFTSKRIATGFIADHPHLLQAPPADDEQGRILDYLADVTVNKPHGVRPKDVAAPIDKLPNIKDLPLPRGSRDRLKQLGPAAFARDLREQDALAVTDTTFRDAHQSLLATRVRSFALKPAAEAVAKLTPELLSVEAWGGATYDVAMRFLFEDPWDRLDELREAMPNVNIQMLLRGRNTVGYTPYPDSVCRAFVKEAASSGVDIFRIFDALNDVSQMRPAIDAVLETNTAVAEVAMAYSGDLSDPNEKLYTLDYYLKMAEEIVKSGAHILAIKDMAGLLRPAAVTKLVTALRREFDLPVHVHTHDTAGGQLATYFAAAQAGADAVDGASAPLSGTTSQPSLSAIVAAFAHTRRDTGLSLEAVSDLEPYWEAVRGLYLPFESGTPGPTGRVYRHEIPGGQLSNLRAQATALGLADRFELIEDNYAAVNEMLGRPTKVTPSSKVVGDLALHLVGAGVDPADFAADPQKYDIPDSVIAFLRGELGNPPGGWPEPLRTRALEGRSEGKAPLTEVPEEEQAHLDADDSKERRNSLNRLLFPKPTEEFLEHRRRFGNTSALDDREFFYGLVEGRETLIRLPDVRTPLLVRLDAISEPDDKGMRNVVANVNGQIRPMRVRDRSVESVTATAEKADSSNKGHVAAPFAGVVTVTVAEGDEVKAGDAVAIIEAMKMEATITASVDGKIDRVVVPAATKVEGGDLIVVVS

SEQ ID NO: 16 - protein, 538 aa, Actinobacillus succinogenes
MTDLNKLVKELNDLGLTDVKEIVYNPSYEQLFEEETKPGLEGFDKGTLTTLGAVAVDTGIFTGRSPKDKYIVCDETTKDTVWWNSEAAKNDNKPMTQETWKSLRELVAKQLSGKRLFVVEGYCGASEKHRIGVRMVTEVAWQAHFVKNMFIRPTDEELKNFKADFTVLNGAKCTNPNWKEQGLNSENFVAFNITEGIQLIGGTWYGGEMKKGMFSMMNYFLPLKGVASMHCSANVGKDGDVAIFFGLSGTGKTTLSTDPKRQLIGDDEHGWDESGVFNFEGGCYAKTINLSQENEPDIYGAIRRDALLENVVVRADGSVDFDDGSKTENTRVSYPIYHIDNIVRPVSKAGHATKVIFLTADAFGVLPPVSKLTPEQTEYYFLSGFTAKLAGTERGVTEPTPTFSACFGAAFLSLHPIQYADVLVERMKASGAEAYLVNTGWNGTGKRISIKDTRGIIDAILDGSIEKAEMGELPIFNLAIPKALPGVDPAILDPRDTYADKAQWQVKAEDLANRFVKNFVKYTANPEAAKLVGAGPKA

SEQ ID NO: 17 - protein, 610 aa, Corynebacterium glutamicum
MTTAAIRGLQGEAPTKNKELLNWIADAVELFQPEAVVFVDGSQAEWDRMAEDLVEAGTLIKLNEEKRPNSYLARSNPSDVARVESRTFICSEKEEDAGPTNNWAPPQAMKDEMSKHYAGSMKGRTMYVVPFCMGPISDPDPKLGVQLTDSEYVVMSMRIMTRMGIEALDKIGANGSFVRCLHSVGAPLEPGQEDVAWPCNDTKYITQFPETKEIWSYGSGYGGNAILAKKCYALRIASVMAREEGWMAEHMLILKLINPEGKAYHIAAAFPSACGKTNLAMITPTIPGWTAQVVGDDIAWLKLREDGLYAVNPENGFFGVAPGTNYASNPIAMKTMEPGNTLFTNVALTDDGDIWWEGMDGDAPAHLIDWMGNDWTPESDENAAHPNSRYCVAIDQSPAAAPEFNDWEGVKIDAILFGGRRADTVPLVTQTYDWEHGTMVGALLASGQTAASAEAKVGTLRHDPMAMLPFIGYNAGEYLQNWIDMGNKGGDKMPSIFLVNWFRRGEDGRFLWPGFGDNSRVLKWVIDRIEGHVGADETVVGHTAKAEDLDLDGLDTPIEDVKEALTAPAEQWANDVEDNAEYLTFLGPRVPAEVHSQFDALKARISAAHA

SEQ ID NO: 18 - protein, 540 aa, Escherichia coli
MRVNNGLTPQELEAYGISDVHDIVYNPSYDLLYQEELDPSLTGYERGVLTNLGAVAVDTGIFTGRSPKDKYIVRDDTTRDTFWWADKGKGKNDNKPLSPETWQHLKGLVTRQLSGKRLFVVDAFCGANPDTRLSVRFITEVAWQAHFVKNMFIRPSDEELAGFKPDFIVMNGAKCTNPQWKEQGLNSENFVAFNLTERMQLIGGTWYGGEMKKGMFSMMNYLLPLKGIASMHCSANVGEKGDVAVFFGLSGTGKTTLSTDPKRRLIGDDEHGWDDDGVFNFEGGCYAKTIKLSKEAEPEIYNAIRRDALLENVTVREDGTIDFDDGSKTENTRVSYPIYHIDNIVKPVSKAGHATKVIFLTADAFGVLPPVSRLTADQTQYHFLSGFTAKLAGTERGITEPTPTFSACFGAAFLSLHPTQYAEVLVKRMQAAGAQAYLVNTGWNGTGKRISIKDTRAIIDAILNGSLDNAETFTLPMFNLAIPTELPGVDTKILDPRNTYASPEQWQEKAETLAKLFIDNFDKYTDTPAGAALVAAGPKL

SEQ ID NO: 19 - protein, 266 aa, Cupriavidus taiwanensis
MLHVSMVGCGAIGRGVLELLKSDPDVVFDVVIVPEHTMDEARGAVSALAPRARVATHLDDQRPDLLVECAGHHALEEHIVPALERGIPCMVVSVGALSEPGMAERLEAAARRGGTQVQLLSGAIGAIDALAAARVGGLDEVIYTGRKPARAWTGTPAEQLFDLEALTEATVIFEGTARDAARLYPKNANVAATVSLAGLGLDRTAVKLLADPHAVENVHHVEARGAFGGFELTMRGKPLAANPKTSALTVFSVVRALGNRAHAVSI

SEQ ID NO: 20 - protein, 267 aa, Polaromonas sp.
MLKIAMIGCGAIGASVLELLHGDSDVVVDRVITVPEARDRTEIAVARWAPRARVLEVLAADDAPDLVVECAGHGAIAAHVVPALERGIPCVVTSVGALSAPGMAQLLEQAARRGKTQVQLLSGAIGGIDALAAARVGGLDSVVYTGRKPPMAWKGTPAEAVCDLDSLTVAHCIFDGSAEQAAQLYPKNANVAATLSLAGLGLKRTQVQLFADPGVSENVHHVAAHGAFGSFELTMRGRPLAANPKTSALTVYSVVRALLNRGRALVI

SEQ ID NO: 21 - protein, 255 aa, Klebsiella pneumoniae
MMKKVMLIGYGAMAQAVIERLPPQVRVEWIVARESHHAAICLQFGQAVTPLTDPLQCGGTPDLVLECASQQAVAQYGEAVLARGWHLAVISTGALADSELEQRLRQAGGKLTLLAGAVAGIDGLAAAKEGGLERVTYQSRKSPASWRGSYAEQLIDLSAVNEAQIFFEGSAREAARLFPANANVAATIALGGIGLDATRVQLMVDPATQRNTHTLHAEGLFGEFHLELSGLPLASNPKTSTLAALSAVRACRELA

SEQ ID NO: 22 - protein, 265 aa, Delftia acidovarans
MNIAVIGCGAIGASVLELLKGHAAVQVGWVLVPEVTDAVRATLALHAPKARAVPALAAEDRPDLIVECAGHAAIEEHVLPALRRGIPAVVASIGALSAPGMAEAVQAAAEAGGTQVQLLSGAIGGVDALAAARIGGLDEVVYTGRKPPMAWTGTPAEQRCDLASLKEAFCIFEGSAREAAQLYPKNANVAATLSLAGMGLDRTTVRLYADPGVDENVHHVAARGAFGSMELTMRGKPLAANPKTSALTVYSVVRAVLNQATAIAI

SEQ ID NO: 23 - protein, 267 aa, Variovorax sp.
MTVRIALIGCGAIGTAALELLREDAGLSVAAIVVPAEAFAATKDVAARLAPGAQVVSAVPADGIDLVVEAAGHAAIEAHVLPALQRGTPCVVASVGALSAQGFAEKLEAAAVAGKTQVQLIAGAIGAIDALAAARIGGLDSVRYTGRKPPHAWKGTPAEQGRDLGALTGATVIFEGSAREAATLYPKNANVAATVSLAGLGLDRTSVRLIADPTVAENVHTVEAEGAFGNFELTMRNKPLAANPKTSALTVYSAVRALRNRVAPLTI

SEQ ID NO: 24 - protein, 266 aa, Comamonas testosteroni
MKNIALIGCGAIGSSVLELLSGDTQLQVGWVLVPEITPAVRETAARLAPQAQLLQALPGDAVPDLLVECAGHAAIEEHVLPALARGIPAVIASIGALSAPGMAERVQAAAETGKTQAQLLSGAIGGIDALAAARVGGLETVLYTGRKPPKAWSGTPAEQVCDLDGLTEAFCIFEGSAREAAQLYPKNANVAATLSLAGLGLDKTMVRLFADPGVQENVHQVEARGAFGAMELTMRGKPLAANPKTSALTVYSVVRAVLNNVAPLAI

SEQ ID NO: 25 - protein, 266 aa, Corynebacterium glutamicum; identical to SEQ ID NO: 24

SEQ ID NO: 26 - protein, 419 aa, Corynebacterium diphtheriae
MSLKDYDAARLAQVREEVTAKYAELKAKNLSLDLTRGKPSAEQLDLSNDLLSLPGGDFRTKDGVDCRNYGGLLGIADIRELWAEALGLPADLVVAQDGSSLNIMFDLISWSYTWGNNDSSRPWSAEEKVKWLCPVPGYDRHFTITEHFGFEMINVPMTDEGPDMGVVRELVKDPQVKGMWTVPVFGNPTGVTFSEQTCRELAEMSTAAPDFRIVWDNAYALHTLSDEFPIVHNVIEFAQAAGNPNRFWFMSSTSKITHAGSGVSFFASSKENIEWYASHANVRGIGPNKLNQLAHAQFFGDVAGLKAHMLKHAASLAPKFERVLEILDSRLSEYGVAKWTSPTGGYFISVDVVPGTASRVVELAKEAGIALTGAGSSFPLHNDPNNENIRLAPSLPPVAELEVAMDGFATCVLMAALEV

SEQ ID NO: 27 - protein, 422 aa, Deinococcus geothermalis
MTKEASRPALDLARQAYEAFKARGLNLNMQRGQPADADFDLSNGLLTVLGAEDVRMDGLDLRNYPGGVAGLPSARALFAGYLDVKAENVLVWNNSSLELQGLVLTFALLHGVRGSTGPWLSQTPKMIVTVPGYDRHFLLLQTLGFELLTVDMQSDGPDVDAVERLAGTDPSVKGILFVPTYSNPGGETISLEKARRLAGLQAAAPDFTIFADDAYRVHHLVEEDRAEPVNFVVLARDAGYPDRAFVFASTSKITFAGAGLGFVASSEDNIRWLSKYLGAQSIGPNKVEQARHVKFLTEYPGGLEGLMRDHAAIIAPKFRAVDEVLRAELGEGGEYATWTLPKGGYFISLDTAEPVADRVVKLAEAAGVSLTPAGATYPAGQDPHNRNLRLAPTRPPVEEVRTAMQVVAACIRLATEEYRAGH

SEQ ID NO: 28 - protein, 435 aa, Mycobacterium tuberculosis
MSFDSLSPQELAALHARHQQDYAALQGMKLALDLTRGKPSAEQLDLSNQLLSLPGDDYRDPEGTDTRNYGGQHGLPGLRAIFAELLGIAVPNLIAGNNSSLELMHDIVAFSMLYGGVDSPRPWIQEQDGIKFLCPVPGYDRHFAITETMGIEMIPIPMLQDGPDVDLIEELVAVDPAIKGMWTVPVFGNPSGVTYSWETVRRLVQMRTAAPDFRLFWDNAYAVHTLTLDFPRQVDVLGLAAKAGNPNRPYVFASTSKITFAGGGVSFFGGSLGNIAWYLQYAGKKSIGPDKVNQLRHLRFFGDADGVRLHMLRHQQILAPKFALVAEVLDQRLSESKIASWTEPKGGYFISLDVLPGTARRTVALAKDVGIAVTEAGASFPYRKDPDDKNIRIAPSFPSVPDLRNAVDGLATCALLAATETLLNQGLASSAPNVR

SEQ ID NO: 29 - protein, 136 aa, Corynebacterium glutamicum
MLRTILGSKIHRATVTQADLDYVGSVTIDADLVHAAGLIEGEKVAIVDITNGARLETYVIVGDAGTGNICINGAAAHLINPGDLVIIMSYLQATDAEAKAYEPKIVHVDADNRIVALGNDLAEALPGSGLLTSRSI

SEQ ID NO: 30 - protein, 1511 aa, Saccharomyces cerevisiae
MSSTDEHIEKDISSRSNHDDDYANSVQSYAASEGQVDNEDLAATSQLSRHLSNILSNEEGIERLESMARVISHKTKKEMDSFEINDLDFDLRSLLHYLRSRQLEQGIEPGDSGIAFKNLTAVGVDASAAYGPSVEEMFRNIASIPAHLISKFTKKSDVPLRNIIQNCTGVVESGEMLFVVGRPGAGCSTFLKCLSGETSELVDVQGEFSYDGLDQSEMMSKYKGYVIYCPELDFHFPKITVKETIDFALKCKTPRVRIDKMTRKQYVDNIRDMWCTVFGLRHTYATKVGNDFVRGVSGGERKRVSLVEAQAMNASIYSWDNATRGLDASTALEFAQAIRTATNMVNNSAIVAIYQAGENIYELFDKTTVLYNGRQIYFGPADKAVGYFQRMGWVKPNRMTSAEFLTSVTVDFENRTLDIKPGYEDKVPKSSSEFEEYWLNSEDYQELLRTYDDYQSRHPVNETRDRLDVAKKQRLQQGQRENSQYVVNYWTQVYYCMIRGFQRVKGDSTYTKVYLSSFLIKALIIGSMFHKIDDKSQSTTAGAYSRGGMLFYVLLFASVTSLAEIGNSFSSRPVIVKHKSYSMYHLSAESLQEIITEFPTKFVAIVILCLITYWIPFMKYEAGAFFQYILYLLTVQQCTSFIFKFVATMSKSGVDAHAVGGLWVLMLCVYAGFVLPIGEMHHWIRWLHFINPLTYAFESLVSTEFHHREMLCSALVPSGPGYEGISIANQVCDAAGAVKGNLYVSGDSYILHQYHFAYKHAWRNWGVNIVWTFGYIVFNVILSEYLKPVEGGGDLLLYKRGHMPELGTENADARTASREEMMEALNGPNVDLEKVIAEKDVFTWNHLDYTIPYDGATRKLLSDVFGYVKPGKMTALMGESGAGKTTLLNVLAQRINMGVITGDMLVNAKPLPASFNRSCGYVAQADNHMAELSVRESLRFAAELRQQSSVPLEEKYEYVEKIITLLGMQNYAEALVGKTGRGLNVEQRKKLSIGVELVAKPSLLLFLDEPTSGLDSQSAWSIVQFMRALADSGQSILCTIHQPSATLFEQFDRLLLLKKGGKMVYFGDIGPNSETLLKYFERQSGMKCGVSENPAEYILNCIGAGATASVNSDWHDLWLASPECAAARAEVEELHRTLPGRAVNDDPELATRFAASYMTQIKCVLRRTALQFWRSPVYIRAKFFECVACALFVGLSYVGVNHSVGGAIEAFSSIFMLLLIALAMINQLHVFAYDSRELYEVREAASNTFHWSVLLLCHAAVENFWSTLCQFMCFICYYWPAQFSGRASHAGFFFFFYVLIFPLYFVTYGLWILYMSPDVPSASMINSNLFAAMLLFCGILQPREKMPAFWRRLMYNVSPFTYVVQALVTPLVHNKKVVCNPHEYNIMDPPSGKTCGEFLSTYMDNNTGYLVNPTATENCQYCPYTVQDQVVAKYNVKWDHRWRNFGFMWAYICFNIAAMLICYYVVRVKVWSLKSVLNFKKWFNGPRKERHEKDTNIFQTVPGDENKITKK

SEQ ID NO: 31 - protein, 944 aa, Saccharomyces cerevisiae
MDTQIAITGVAVGKEINNDNSKTDQKVSLPKADVPCIDKATQTIIEGCSKDDPRLSYPTKLETTEKGKTKRNSFACVCCHSLKQKCEPSDVNDIYRKPCRRCLKHKKLCKFDLSKRTRKRKPRSRSPTPFESPMVNVSTKSKGPTDSEESSLKDGTSYLASFPSDPNAKQFPNSRTVLPGLQQSLSDLWSTLSQPPSYGAREAETTSTGEITTNNHTKSNGSVPTNPAVLASNDEHTNISDAPVIYSTYNSPVPISSAPTSINSEALFKHRPKIVGDEETQNVKVKRQKKSYSRHMTRSFRKQLQSLIISQKGKIRDISMKLDTWSKQWNDLVEKSMFLPTIADPVSVGIISHEEATLRLHLYKTEISYLSKLPFIKVEENVSVDELRKKKPILFSVIMSCVSIVLTPKQTTRGTIMKLDSFVLNLITNQIFKANNKSIEIIESLSTLCLWYNFFEWSSKTRYHIFNYICCCLTRDLGPTYVNRSFGMFSDEDPKRFKSPLELYSNGASLTLLVYISALNISIFLRQSIQARWSHVTEKACEDLVKETKKSRHYDNDKLLLDSADDPILVQFAKMNHVLENIHTHLHERDLNDDEFDDPIFTKKYLNKLMEKYHKQLQEIFTKLDRNRPRVIAFYYSVEAYLYQYKLAVFIGEMSHTINEKVELPREIMDDFVKCYHCCKSALEEFSKLEPILITSLPLFHTSRIIYTVGMLLLKLRYSVVAIPSFHDLMPLTDDAIALVIGVNNLLEKTSELYPFNNSLYKFRYVIALFCQTYANKVIDVADRYNAEREKLKEKQVIDEVSNGHDGTKPINAYVTESQKMPTEEDPIIDNNTNQNITAVPDEMLPVYSRVRDDTAAMNLNINSTSYMNESPHEHRESMTGTTLLPPPFISNDVTNSADSTNIKPSPSSSVDNLNDYLTDINSLAWGVNSLNDEFWTDLFMNDI

SEQ ID NO: 32 - protein, 438 aa, Schizosaccharomyces pombe
MGELKEILKQRYHELLDWNVKAPHVPLSQRLKHFTWSWFACTMATGGVGLIIGSFPFRFYGLNTIGKIVYILQIFLFSLFGSCMLFRFIKYPSTIKDSWNHHLEKLFIATCLLSISTFIDMLAIYAYPDTGEWMVWVIRILYYIYVAVSFIYCVMAFFTIFNNHVYTIETASPAWILPIFPPMICGVIAGAVNSTQPAHQLKNMVIFGILFQGLGFWVYLLLFAVNVLRFFTVGLAKPQDRPGMFMFVGPPAFSGLALINIARGAMGSRPYIFVGANSSEYLGFVSTFMAIFIWGLAAWCYCLAMVSFLAGFFTRAPLKFACGWFAFIFPNVGFVNCTIEIGKMIDSKAFQMFGHIIGVILCIQWILLMYLMVRAFLVNDLCYPGKDEDAHPPPKPNTGVLNPTFPPEKAPASLEKVDTHVTSTGGESDPPSSEHESV

SEQ ID NO: 33 - protein, 267 aa, Artificial Sequence
  Synthetic Sequence
MLHIAMIGCGAIGAGVLELLKSDPDLRVDAVIVPEESMDAVREAVAALAPVARVLTALPADARPDLLVECAGHRAIEEHVVPALERGIPCAVASVGALSEPGLAERLEAAARRGGTQVQLLSGAIGAIDALAAARVGGLDSVVYTGRKPPLAWKGTPAEQVCDLDALTEATVIFEGSAREAARLYPKNANVAATLSLAGLGLDRTQVRLIADPAVTENVHHVEARGAFGGFELTMRGKPLAANPKTSALTVYSVVRALGNRAHALSI

SEQ ID NO: 34 - protein, 267 aa, Pseudomonas aeruginosa
MLNIVMIGCGAIGAGVLELLENDPQLRVDAVIVPRDSETQVRHRLASLRRPPRVLSALPAGERPDLLVECAGHRAIEQHVLPALAQGIPCLVVSVGALSEPGLVERLEAAAQAGGSRIELLPGAIGAIDALSAARVGGLESVRYTGRKPASAWLGTPGETVCDLQRLEKARVIFDGSAREAARLYPKNANVAATLSLAGLGLDRTQVRLIADPESCENVHQVEASGAFGGFELTLRGKPLAANPKTSALTVYSVVRALGNHAHAISI

SEQ ID NO: 35 - protein, 885 aa, Artificial Sequence
  Synthetic Sequence
MNEQYSALRSNVSMLGKLLGDTIKDALGEDILDRVETIRKLSKSSRAGNEANRQELLTTLQNLSNDELLPVARAFSQFLNLTNTAEQYHSISPHGEAHASNPEAIARLFRKLKDQPNLSEADIKKAVESLSIELVLTAHPTEITRRTLIHKLVEVNTCLSQLDHDDLADYERNQIMRRLRQLIAQSWHTDEIRKQRPTPVDEAKWGFAVVENSLWEGVPAFLREFNEQLEESLGYKLPVEAVPVRFTSWMGGDRDGNPNVTAEITRHVLLLSRWKAADLFLKDIQVLVSELSMVECTPELRALAGEEGALEPYRELLKNLRTQLMATLAYLEARLKGERLRPKPAGLLTQNEQLWEPLYACYQSLQACGMGIIANGQLLDTLRRVKCFGVPLVRIDIRQESTRHTEALAELTRYLGLGDYESWSEADKQAFLIRELNSKRPLLPRNWEPSAETQEVLDTCKVIAEAPKGSIAAYVISMAKTPSDVLAVHLLLKEAGCPFALPVAPLFETLDDLNNAEDVMTQLLNIDWYRGFIQGKQMVMIGYSDSAKDAGVMAASWAQYRAQDALIKTCEKAGIELTLFHGRGGSIGRGGAPAHAALLSQPPGSLKGGLRVTEQGEMIRFKLGLPEVTVSSLSLYASAILEANLLPPPEPKDEWRDIMDELSDISCDLYRGYVRENKDFVPYFRAATPEQELGKLPLGSRPAKRRPTGGVESLRAIPWIFAWTQNRLMLPAWLGAGAALQKVIEDGKQDELEAMCRDWPFFSTRIGMLEMVFAKADLWLAEYYDQRLVDKALWPLGEELRDQLEEDIKVVLAIANDSHLMADLPWIAESIALRNVYTDPLNVLQAELLHRSRQQEEEGKEPDPRVEQALMVTIAGIAAGMRNTG

SEQ ID NO: 36 - protein, 245 aa, Artificial Sequence
  Synthetic Sequence
MSLQDFDADELAAFHEDIKAAYEELKAKNLKLDLTRGKPSSEQLDFSNELLALPGEDDYRDADGTDTRNYGGLTGIPEIRAIWAELLGIPVENVLAGDASSLNIMFDLISWSYLFGNNDSPRPWSQEEVVKWLCPVPGYDRHFAITESFGIEMIPVPLREDGPDMDAVEELVAKDPQIKGMWTVPVFSNPTGATYSEEVARRLAEMETAAPDFRIVWDNAYAVHTLTDEFPEVIDILGLAEAAGN

SEQ ID NO: 37 - protein, 127 aa, Bacillus subtilis
MYRTMMSGKLHRATVTEANLNYVGSITIDEDLIDAVGMLPNEKVQIVNNNNGARLETYIIPGKRGSGVICLNGAAARLVQEGDKVIIISYKMMSDQEAASHEPKVAVLNDQNKIEQMLGNEPARTIL

SEQ ID NO: 38 - protein, 540 aa, Tribolium castaneum
MPATGEDQDLVQDLIEEPATFSDAVLSSDEELFHQKCPKPAPIYSPISKPVSFESLPNRRLHEEFLRSSVDVLLQEAVFEGTNRKNRVLQWREPEELRRLMDFGVRGAPSTHEELLEVLKKVVTYSVKTGHPYFVNQLFSAVDPYGLVAQWATDALNPSVYTYEVSPVFVLMEEVVLREMRAIVGFEGGKGDGIFCPGGSIANGYAISCARYRFMPDIKKKGLHSLPRLVLFTSEDAHYSIKKLASFEGIGTDNVYLIRTDARGRMDVSHLVEEIERSLREGAAPFMVSATAGTTVIGAFDPIEKIADVCQKYKLWLHVDAAWGGGALVSAKHRHLLKGIERADSVTWNPHKLLTAPQQCSTLLLRHEGVLAEAHSTNAAYLFQKDKFYDTKYDTGDKHIQCGRRADVLKFWFMWKAKGTSGLEKHVDKVFENARFFTDCIKNREGFEMVIAEPEYTNICFWYVPKSLRGRKDEADYKDKLHKVAPRIKERMMKEGSMMVTYQAQKGHPNFFRIVFQNSGLDKADMVHFVEEIERLGSDL

SEQ ID NO: 39 - protein, 128 aa, Artificial Sequence
  Synthetic Sequence
MLRTMLKSKIHRATVTQADLHYVGSVTIDADLLDAADILEGEKVAIVDITNGARLETYVIAGERGSGVIGINGAAAHLVHPGDLVIIIAYAQMSDAEARAYEPRVVFVDADNRIVELGNDPAEALPGG

SEQ ID NO: 40 - protein, 585 aa, Artificial Sequence
  Synthetic Sequence
MPANGNFPVALEVISIFKPYNSAVEDLASMAKTDTSASSSGSDSAGSSEDEDVQLFASKGNLLNSKLLKKSNNNNKNNNINENNNKNAAAGLKRFASLPNRAEHEEFLRDCVDEILKLAVFEGTNRSSKVVEWHDPEELKKLFDFELRAEPDSHEKLLELLRATIRYSVKTGHPYFVNQLFSSVDPYGLVGQWLTDALNPSVYTYEVAPVFTLMEEVVLREMRRIVGFPNDGEGDGIFCPGGSIANGYAISCARYKYAPEVKKKGLHSLPRLVIFTSEDAHYSVKKLASFMGIGSDNVYKIATDEVGKMRVSDLEQEILRALDEGAQPFMVSATAGTTVIGAFDPLEGIADLCKKYNLWMHVDAAWGGGALMSKKYRHLLKGIERADSVTWNPHKLLAAPQQCSTFLTRHEGILSECHSTNATYLFQKDKFYDTSYDTGDKHIQCGRRADVLKFWFMWKAKGTSGFEAHVDKVFENAEYFTDSIKARPGFELVIEEPECTNICFWYVPPSLRGMERDNAEFYEKLHKVAPKIKERMIKEGSMMITYQPLRDLPNFFRLVLQNSGLDKSDMLYFINEIERLGSDLV

SEQ ID NO: 41 - protein, 273 aa, Corynebacterium glutamicum
MLSGNGQLDANKWTPFINSQTWTTYILPGLWGTLKSAVFSVILALVMGTALGLGRISEIRILRWFCAVIIETFRAIPVLILMIFAYQMFAQYNIVPSSQLAFAAVVFGLTMYNGSVIAEILRSGIASLPKGQKEAAIALGMSSRQTTWSILLPQAVAAMLPALISQMVIALKDSALGYQIGYIEVVRSGIQSASVNRNYLAALFVVALIMIVLNFSLTALASRIERQLRAGRARKNIVAKVPEQPDQGLETKDNVNVDWQDPDYKDLKTPGVQ

SEQ ID NO: 42 - protein, 242 aa, Corynebacterium glutamicum
MIKMTGVQKYFGDFHALTDIDLEIPRGQVVVVLGPSGSGKSTLCRTINRLETIEEGTIEIDGKVLPEEGKGLANLRADVGMVFQSFNLFPHLTIKDNVTLAPIKVRKMKKSEAEKLAMSLLERVGIANQADKYPAQLSGGQQQRVAIARALAMNPKIMLFDEPTSALDPEMVNEVLDVMASLAKEGMTMVCVTHEMGFARKAADRVLFMADGLIVEDTEPDSFFTNPKSDRAKDFLGKILAH

SEQ ID NO: 43 - protein, 228 aa, Corynebacterium glutamicum
MSTLWADLGPSLLPAFWVTIKLTIYSAIGAMIFGTILTTMRVSPVKILRTLSTAYINTVRNTPLTLVVLFCSFGLYQNLGLTLAGRESSTFLVDNNFRLAVLGFILYTSTFVAESLRSGINTVHFGQAEAARSLGLGFGATFRSIIFPQAVRAAIVPLGNTLIALTKNTTIASVIGVGEASLLMKATIENHANMLFVVFAIFAVGFMILTLPMGLGLGKLSERLAVKK

SEQ ID NO: 44 - protein, 260 aa, Homo sapiens
MSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 45 - protein, 330 aa, Flaveria bidentis
MSAASAFAMNAPSFVNASSLKKASTSARSGVLSARFTCNSSSSSSSSATPPSLIRNEPVFAAPAPIITPNWTEDGNESYEEAIDALKKTLIEKGELEPVAATRIDQITAQAAAPDTKAPFDPVERIKSGFVKFKTEKFVTNPALYDELAKGQSPKFMVFACSDSRVCPSHVLDFQPGEAFVVRNVANMVPPFDKTKYSGVGAAVEYAVLHLKVQEIFVIGHSRCGGIKGLMTFPDEGPHSTDFIEDWVKVCLPAKSKVVAEHNGTHLDDQCVLCEKEAVNVSLGNLLTYPFVRDGLRNKTLALKGGHYDFVNGTFELWALDFGLSSPTSV

SEQ ID NO: 46 - protein, 221 aa, Saccharomyces cerevisiae
MSATESSSIFTLSHNSNLQDILAANAKWASQMNNIQPTLFPDHNAKGQSPHTLFIGCSDSRYNENCLGVLPGEVFTWKNVANICHSEDLTLKATLEFAIICLKVNKVIICGHTDCGGIKTCLTNQREALPKVNCSHLYKYLDDIDTMYHEESQNLIHLKTQREKSHYLSHCNVKRQFNRIIENPTVQTAVQNGELQVYGLLYNVEDGLLQTVSTYTKVTPK

SEQ ID NO: 47 - protein, 281 aa, Candida albicans
MGRENILKYQLEHDHESDLVTEKDQSLLLDNNNNLNGMNNTIKTHPVRVSSGNHNNFPFTLSSESTLQDFLNNNKFFVDSIKHNHGNQIFDLNGQGQSPHTLWIGCSDSRAGDQCLATLPGEIFVHRNIANIVNANDISSQGVIQFAIDVLKVKKIIVCGHTDCGGIWASLSKKKIGGVLDLWLNPVRHIRAANLKLLEEYNQDPKLKAKKLAELNVISSVTALKRHPSASVALKKNEIEVWGMLYDVATGYLSQVEIPQDEFEDLFHVHDEHDEEEYNPH

SEQ ID NO: 48 - protein, 192 aa, Porphyromonas gingivalis
MAQRENSDYLTTKMALIQSVRGFTPIIGEDTFLAENATIVGDVVMGKGCSVWFNAVLRGDVNSIRIGDNVNIQDGSILHTLYQKSTIEIGDNVSVGHNVVIHGAKICDYALIGMGAVVLDHVVVGEGAIVAAGSVVLTGTQIEPNSIYAGAPARFIKKVDPEQSREMNFRIAHNYRMYASWFKDESSEIDNP

SEQ ID NO: 49 - protein, 207 aa, Mycobacterium tuberculosis
MPNTNPVAAWKALKEGNERFVAGRPQHPSQSVDHRAGLAAGQKPTAVIFGCADSRVAAEIIFDQGLGDMFVVRTAGHVIDSAVLGSIEYAVTVLNVPLIVVLGHDSCGAVNAALAAINDGTLPGGYVRDVVERVAPSVLLGRRDGLSRVDEFEQRHVHETVAILMARSSAISERIAGGSLAIVGVTYQLDDGRAVLRDHIGNIGEEV

SEQ ID NO: 50 - protein, 219 aa, Escherichia coli
MKEIIDGFLKFQREAFPKREALFKQLATQQSPRTLFISCSDSRLVPELVTQREPGDLFVIRNAGNIVPSYGPEPGGVSASVEYAVAALRVSDIVICGHSNCGAMTAIASCQCMDHMPAVSHWLRYADSARVVNEARPHSDLPSKAAAMVRENVIAQLANLQTHPSVRLALEEGRIALHGWVYDIESGSIAAFDGATRQFVPLAANPRVCAIPLRQPTAA

SEQ ID NO: 51 - protein, 220 aa, Escherichia coli
MKDIDTLISNNALWSKMLVEEDPGFFEKLAQAQKPRFLWIGCSDSRVPAERLTGLEPGELFVHRNVANLVIHTDLNCLSVVQYAVDVLEVEHIIICGHYGCGGVQAAVENPELGLINNWLLHIRDIWFKHSSLLGEMPQERRLDTLCELNVMEQVYNLGHSTIMQSAWKRGQKVTIHGWAYGIHDGLLRDLDVTATNRETLEQRYRHGISNLKLKHANHK

SEQ ID NO: 52 - protein, 450 aa, Clostridium symbiosum
MSKYVDRVIAEVEKKYADEPEFVQTVEEVLSSLGPVVDAHPEYEEVALLERMVIPERVIEFRVPWEDDNGKVHVNTGYRVQFNGAIGPYKGGLRFAPSVNLSIMKFLGFEQAFKDSLTTLPMGGAKGGSDFDPNGKSDREVMRFCQAFMTELYRHIGPDIDVPAGDLGVGAREIGYMYGQYRKIVGGFYNGVLTGKARSFGGSLVRPEATGYGSVYYVEAVMKHENDTLVGKTVALAGFGNVAWGAAKKLAELGAKAVTLSGPDGYIYDPEGITTEEKINYMLEMRASGRNKVQDYADKFGVQFFPGEKPWGQKVDIIMPCATQNDVDLEQAKKIVANNIKYYIEVANMPTTNEALRFLMQQPNMVVAPSKAVNAGGVLVSGFEMSQNSERLSWTAEEVDSKLHQVMTDIHDGSAAAAERYGLGYNLVAGANIVGFQKIADAMMAQGIAW

SEQ ID NO: 53 - protein, 447 aa, Corynebacterium glutamicum
MTVDEQVSNYYDMLLKRNAGEPEFHQAVAEVLESLKIVLEKDPHYADYGLIQRLCEPERQLIFRVPWVDDQGQVHVNRGFRVQFNSALGPYKGGLRFHPSVNLGIVKFLGFEQIFKNSLTGLPIGGGKGGSDFDPKGKSDLEIMRFCQSFMTELHRHIGEYRDVPAGDIGVGGREIGYLFGHYRRMANQHESGVLTGKGLTWGGSLVRTEATGYGCVYFVSEMIKAKGESISGQKIIVSGSGNVATYAIEKAQELGATVIGFSDSSGWVHTPNGVDVAKLREIKEVRRARVSVYADEVEGATYHTDGSIWDLKCDIALPCATQNELNGENAKTLADNGCRFVAEGANMPSTPEAVEVFRERDIRFGPGKAANAGGVATSALEMQQNASRDSWSFEYTDERLQVIMKNIFKTCAETAAEYGHENDYVVGANIAGFKKVADAMLAQGVI

SEQ ID NO: 54 - protein, 421 aa, Peptoniphilus asaccharolyticus
MTDTLNPLVAAQEKVRIACEKLGCDPAVYELLKEPQRVIEISIPVKMDDGTVKVFKGWRSAHSSAVGPSKGGVRFHPNVNMDEVKALSLWMTFKGGALGLPYGGGKGGICVDPAELSERELEQLSRGWVRGLYKYLGDRIDIPAPDVNTNGQIMSWFVDEYVKLNGERMDIGTFTGKPVAFGGSEGRNEATGFGVAVVVRESAKRFGIKMEDAKIAVQGFGNVGTFTVKNIERQGGKVCAIAEWDRNEGNYALYNENGIDFKELLAYKEANKTLIGFPGAERITDEEFWTKEYDIIVPAALENVITGERAKTINAKLVCEAANGPTTPEGDKVLTERGINLTPDILTNSGGVLVSYYEWVQNQYGYYWTEAEVEEKQEADMMKAIKGVFAVADEYNVTLREAVYMYAIKSIDVAMKLRGWY